SEQUENCE LISTING (2 sequences)

SEQ ID NO: 1 - DNA, 10 bp, Artificial Sequence
  Chemically synthesized
agcgtgactt

SEQ ID NO: 2 - protein, 5 aa, Artificial Sequence
  Chemically synthesized
  MOD_RES LOCATION: (4)..(4): D-Phe
  MOD_RES LOCATION: (5)..(5): Lys(N3)
RGDXX

(19) United States
(12) Patent Application Publication
(10) Pub. No.: US 2023/0174690 A1
(43) Pub. Date: Jun. 8, 2023

(54) COMPOSITIONS AND METHODS FOR SINGLE-STEP MULTIPURPOSE SURFACE FUNCTIONALIZATION

(71) Applicant: Rutgers, The State University of New Jersey, New Brunswick, NJ (US)

(72) Inventors: Enver Izgu, Piscataway, NJ (US); Zhaojun Jia, Piscataway, NJ (US)

(21) Appl. No.: 17/789,057

(22) PCT Filed: Dec. 14, 2020
(86) PCT No.: PCT/US2020/064791

§ 371 Date: Jun. 24, 2022

Related U.S. Application Data
(60) Provisional application No. 62/953,709, filed on Dec. 26, 2019.

(51) Int. Cl. C08F 38/02; C08F 138/02

(52) U.S. Cl. CPC C08F 38/02; C08F 138/02

(57) ABSTRACT

Compositions and methods for functionalizing a variety of surfaces are provided herein. The compositions include compounds of formula (I), which react with azido compounds (R-N₃) to form cycloadducts that can spontaneously polymerize on a surface. The R-group in the azido compound can be any molecule of interest, including small molecules and macromolecules

### STATEMENT REGARDING FEDERALLY SPONSORED RESEARCH OR DEVELOPMENT

This invention was made with government support under EB029548 awarded by the National Institutes of Health. The government has certain rights in the invention.

### CROSS-REFERENCE TO RELATED APPLICATIONS

The present application claims priority under 35 U.S.C. § 119(e) to U.S. Provisional Application No. 62/953,709, filed Dec. 26, 2019, which is hereby incorporated by reference herein in its entirety.

### BACKGROUND

The demand for functional materials has recently surged in biotechnology and medicine, where applications such as biomaterials, diagnostics, and pharmaceutics require advanced material functionality. Surface functionalization allows for the control of a wide array of material properties, such as wettability, chemical stability, biocompatibility, catalytic activity, sensing, antifouling resistance, antimicrobial resistance, and cell affinity. Despite the great interest in controlling these properties, a surface functionalization technology that is simple, robust, and also generalizable has yet to be established.

One of Nature’s most effective adhesives is the mussel foot protein, whose remarkably strong underwater adhesion stems from its 3,4-dihydroxy-phenylalanine (L-DOPA) component. Sessile mussels use L-DOPA to adhere to salt-encrusted, slimy surfaces (e.g., wood and stones). Research centered on mussel-inspired surface coating has shed light on the polymerization and metal coordination of catechols, including dopamine, L-DOPA, and other phenolic analogs. They form adherent polymeric coatings through oxidative self-polymerization at near-neutral or basic pH. They create crosslinked supramolecular networks of diverse forms through coordination with transition metal ions, for instance, Fe³⁺ and Cu²⁺. Catecholamine polymerization provides strong and largely material-independent substrate adhesion, thus it has been widely embraced as a powerful method for surface grafting.

Currently practiced DOPA-mediated surface functionalization techniques employ a stepwise approach where the material surface is first deposited with a polymeric layer, followed by the addition of MOIs (molecules of interest) with reactive groups (e.g., amines, thiols). This process may be straightforward, but has limitations with regard to site specificity, speed, adaptability, and broader applications. Consequently, there is a need in the art for faster, more adaptable, and simpler methods of functionalizing surfaces with MOIs. The present disclosure addresses this need.

### BRIEF SUMMARY OF THE INVENTION

The present disclosure relates in part to compositions and methods for functionalizing a variety of surfaces. Compositions of the present disclosure include compounds of formula (I), or a salt, solvate, stereoisomer, tautomer, or any mixtures thereof, wherein the substituents in (I) are defined elsewhere herein:

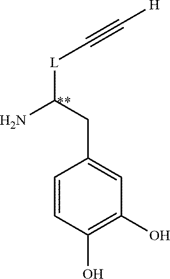

In certain aspects the present disclosure further relates to a single-step method for the multipurpose functionalization of diverse surfaces. In certain embodiments, the method comprises contacting at least a portion of a surface with a composition comprising the compound of formula (I), a copper (II) salt, a copper (I) ligand, and an azido compound (R—N₃), wherein R comprises a chromophore, fluorogenic molecule, oligonucleotide, nucleic acid, polyethylene glycol, peptide, polypeptide, protein, therapeutic agent, or lipid, wherein at least a portion of the surface is coated with the reaction product of the compound of formula (I) and the azido compound (R—N₃).

### BRIEF DESCRIPTION OF THE FIGURES

The drawings illustrate generally, by way of example, but not by way of limitation, various embodiments of the present application.

FIGS. 1A-1B provide an overview of the surface functionalization chemistry of the present disclosure. FIG. 1A shows the chemical synthesis of p-DOPAmide, wherein the linker comprises —(CH₂CH₂O)₂CH₂—. FIG. 1B shows the mechanism and characteristics of site-specific single-step functionalization of material surfaces.

FIGS. 2A-2C show single-step drop-coating functionalization of surfaces. Ti: titanium, Si: silicon, Ge: germanium, PTFE: polytetrafluoroethylene, PEEK: polyether ether ketone, PC: polycarbonate, PU: polyurethane, SiR: silicon rubber. Stacked confocal fluorescence images correlate with surface population of coumarin. FIG. 2A provides surfaces coated with 3-N₃-7-hydroycoumarin as the MOI-N₃, wherein water droplets on the surface of bare or coated surfaces indicate the change in wettability and hydrophobicity. FIG. 2B provides (i) uncoated materials; materials drop-coated using the MCM: (ii) without the MOI-N₃; (iii) with TAMRA-N₃ as the MOI-N₃; (iv) with 3-N₃-7-hydroxycoumarin as the MOI-N₃; and (v) fluorescence images of 3-N₃-7-hydroxycoumarin as the MOI-N₃. Materials shown in (ii), (iii), and (iv) were incubated at 37° C. for 4 h. Coated materials were washed, in some cases also sonicated, before the surface characterization. The sizes of substrates: Ti/TiO2 and Si/SiO₂: 10 mm in diameter; glass: 18 mm × 18 mm; PTFE, PEEK, PC, and nylon: 15-20 mm, and SiR: 5 mm in diameter. FIG. 2C provides contact angle measurements for regular surfaces: (i) uncoated surfaces; (ii) surfaces coated without a MOI-N₃; and (iii) surfaces coated with 3-N₃-7-hydroxycoumarin as the MOI-N₃.

FIGS. 3A-3C show single-step drop-coating functionalization of titanium with a 10 nucleotide long 5′-N₃-3′-FAM (fluorescein) as the MOI-N₃. FIG. 3A shows a surface analysis performed by fluorescence microscopy at 488 nm excitation. FIGS. 3B-3C show surface analysis by atomic force microscopy (AFM).

FIG. 4 provides XPS survey spectra of Ti/TiO2 (i) before and (ii) after coating using the MCM (MOI-N₃ = 3-N₃-7-hydroxycoumarin).

FIGS. 5A-5R provide XPS survey spectra before and after 3-N₃-7-hydroxycoumarin coating. Black dots denote substrate peaks (for PEEK, PC, and nylon, no substrate peaks were identified; instead, N 1s or O 1s served as a substrate peak for ease of comparison with the coated materials). FIG. 5A: Ti/TiO₂ before coating. FIG. 5B: Ti/TiO₂ after coating. FIG. 5C: Si/SiO₂ before coating. FIG. 5D: Si/SiO₂ after coating. FIG. 5E: Ge before coating.

FIG. 5F: Ge after coating. FIG. 5G: Glass before coating. FIG. 5H: Glass after coating.

FIG. 5I: PTFE before coating. FIG. 5J: PTFE after coating. FIG. 5K: PEEK before coating.

FIG. 5L: PEEK after coating. FIG. 5M: PC before coating. FIG. 5N: PC after coating. FIG. 5O: Nylon before coating. FIG. 5P: Nylon after coating. FIG. 5Q: SiR before coating. FIG. 5R: SiR after coating.

FIG. 6 provides AFM scratch images showing topography and thickness of a Si/SiO₂ substrate, which was drop coated using the MCM (MOI-N₃ = N₃-DNA-FAM) with an incubation period of 4 h. Inset: Height profile along the substrate/coating edge (diagonal line).

FIG. 7 provides an ATR-FTIR spectra overlay of Ti/TiO₂ surfaces: (i) coated with p-DOPAmide, Cu(II), and THPTA, (ii) coated with p-DOPAmide, Cu(II), THPTA, and 3-N₃-7-hydroxycoumarin, and (iii) deposited with reference compound S4.

FIG. 8 provides an XPS survey spectral overlay showing the change in Ti 2p XPS signals over time when the Ti/TiO₂ surface was drop coated with the MCM wherein MOI-N₃ is 3-N₃-7-hydroxycoumarin. The original substrate signals (468-456 eV range) decreased significantly within the first 30 mins.

FIG. 9A provides images of the polymerization of p-DOPAmide in MCMs (MOI-N₃ = 3-N₃-7-hydroxycoumarin) with various p-DOPAmide concentrations and reaction times. FIG. 9B provides confocal fluorescence microscopy images showing the effects of (i) p-DOPAmide concentration (incubation time of 2 h at each concentration) and (ii) incubation time (concentration of p-DOPAmide 50 mM at each time point) on the coumarin density on Ti/TiO₂ surfaces.

FIGS. 10A-10B provide images of the oxidative polymerization of p-DOPAmide (i) in the absence of any additive, (ii) in the presence of THPTA, (iii) in the presence of CuSO₄, and (iv) in the presence of both CuSO₄ and THPTA. Buffers used: MES for pH 5.5, PBS for pH 7.4, and Tris for pH 8.5. Solutions of p-DOPAmide, additives, and buffers were not (FIG. 10A) or were (FIG. 10B) bubbled with N₂ for 15 min prior to mixing.

FIG. 11 provides Raman spectra of Ti/TiO₂ treated with coating mixtures with different combinations of Cu (5 mM), ligand (10 mM), and p-DOPAmide (10 mM), wherein

Cu = CuSO₄ and ligand = THPTA. Black dots indicate substrate signals belong to TiO₂, while the prismatic indicates bands from p-DOPAmide and its oxidative products: catecholic C-OH (1335 cm⁻¹) and aromatic C-C stretching (1580 cm⁻¹). To fit the special line into the graph, the intensity of p-DOPAmide-Cu-ligand spectrum has been kept at 20%.

FIG. 12 provides confocal fluorescence microscopy images (insets are ambient light appearances) showing the effects of additives and catechol O-protection on coating and grafting of Ti/TiO₂. The Ti/TiO₂ surfaces were prepared: (i) with a coating mixture containing p-DOPAmide, CuSO₄, THPTA, and 3-N₃-7-hydroxycoumarin; (ii) with the mixture of (i) further comprising FeC13 (0.05 eq); (iii) with the mixture of (i) further comprising FeC13 (0.5 eq); and (iv) with the mixture of (i) further comprising compound S3.

FIG. 13 provides an XPS analysis of coatings showing the effects of additives and catechol O-protection on coating and grafting of Ti/TiO₂. The Ti/TiO₂ surfaces were prepared: (i) with a coating mixture containing p-DOPAmide, CuSO₄, THPTA, and 3-N₃-7-hydroxycoumarin; (ii) with the mixture of (i) further comprising FeC13 (0.05 eq); (iii) with the mixture of (i) further comprising FeC13 (0.5 eq); and (iv) with the mixture of (i) further comprising compound S3.

FIG. 14 provides the results of a MicroBCA assay of Cu(I) production using different media: (i) Cu, (ii) ligand, (iii) Cu + ligand, (iv) p-DOPAmide, (v) p-DOPAmide + ligand, (vi) pDOPAmide + Cu, (vii) p-DOPAmide + Cu + ligand, and (viii) compound S3 + Cu + ligand, wherein Cu = CuSO₄ (5 mM), ligand = THPTA; and S3 is a p-DOPAmide derivative with catechol O-protection.

FIG. 15A provides the design and observations of the MicroBCA assay using different media. Cu = CuSO₄, Lig = THPTA. FIG. 15B provides a summary of the compositions of the final mixtures.

FIG. 16 provides ambient light images (upper) and fluorescence images (lower; inset with tile scans) of Ti/TiO₂ and Si/SiO₂ substrates treated with (i) CuSO₄ and TAMRA-N₃, (ii) THPTA and TAMRA-N₃, (iii) CuSO₄, THPTA, and TAMRA-N₃, (iv) p-DOPAmide and TAMRA-N₃, (v) p-DOPAmide, THPTA, and TAMRA-N₃, (vi) p-DOPAmide, CuSO₄, and TAMRA-N₃, and (vii) p-DOPAmide, CuSO₄, THPTA, and TAMRA-N₃.

FIGS. 17A-17D provide images of interfacial film formation during drop coating (FIG. 17A and FIG. 17C) using p-DOPAmide, CuSO₄, THPTA, and 3-N₃-7-hydroxycoumarin, with dip coated samples (FIG. 17B and FIG. 17D) as controls. A thin film (indicated by wrinkles) developed along the meniscus of the domical droplet dropped on the substrates, which encapsulated the droplet liquid and prevented its leaching even if the sample was tilted (FIG. 17A) or inverted (FIG. 17C), which was not the case for dip coated substrates (FIG. 17B and FIG. 17D).

FIGS. 18A-18B provide images showing film formation on different materials during drop-coating using coating mixtures without (FIG. 18A) or with (FIG. 18B) 3-N₃-7-hydroxycoumarin.

FIGS. 19A-19D provide images showing the effects of interfacial film formation on coating-grafting using p-DOPAmide, CuSO₄, THPTA, and TAMRA-N₃. Arrows indicate the liquid/air interface. FIG. 19A: drop coated Ti/TiO₂ column (5 µL mixture volume). FIG. 19B: drop coated Ti/TiO₂ column (100 µL mixture volume). FIG. 19C: dip coated Ti/TiO₂ column (500 µL mixture volume). FIG. 19D: dip coated quartz cuvette (250 µL mixture volume).

FIG. 20 provides images showing interfacial oxidative p-DOPAmide self-assembly in a quartz cuvette. Interfacial behaviors of 3-N₃-7-hydroxycoumarin-containing mixtures with and without p-DOPAmide (10 mM, 4 h).

FIG. 21A: Ti/TiO₂ substrate that was drop coated with 100 µL of the MCM (MOI-N₃ = 3-N₃-7-hydroxycoumarin). FIG. 21B: The droplet of FIG. 21A after incubation at 37° C. for 1 h, resulting in formation of a light-reflecting polymeric thin film (arrow). FIG. 21C: The droplet of FIG. 21B showing a wrinkled morphology with a light-reflecting property (arrow) after aspirating the liquid with a pipette (disrupted site is indicated by an asterisk (*). FIG. 21D: Small debris from the film floated on water that was used to rinse the Ti/TiO₂ substrate. FIG. 21E: the washing solution dropped on a glass coverslip, which contained the film debris and some brown-colored deposits. FIG. 21F: Air dried coverslip.

FIGS. 22A-22D: Examination of film debris by CLSM: (FIG. 22A) brightfield image, (FIG. 22B) fluorescence image, (FIG. 22C) merged, and (FIG. 22D) z-stack image.

FIG. 23A shows AFM examination of surface-adhered film debris (white arrows) on drop coated Ti/TiO₂ substrate. FIG. 23B shows a 3D visualization of the AFM examination of surface-adhered film debris (white arrows) on drop coated Ti/TiO₂ substrate.

FIG. 24A provides TEM images of a film formed during drop coating of 3-N₃-7-hydroxycoumarin onto a Si/SiO₂ substrate. FIG. 24B provides a further magnified image of the film formed during drop coating of 3-N₃-7-hydroxycoumarin onto a Si/SiO₂ substrate.

FIG. 25A provides an optical image of film debris deposited on a Si/SiO₂ substrate. FIG. 25B provides analysis of the film debris deposited on a Si/SiO₂ substrate by Raman spectroscopy, wherein black dots indicate substrate signals belonging to Si/SiO₂ and prismatic symbol indicates bands from p-DOPAmide and its oxidative products. Indications: (i) substrate, (ii) film, and (iii) film-adhered particles.

FIGS. 26A-26B show the investigation of interfacial film formation on substrate with regard to coating mixture compositions and incubation time: experimental design (FIG. 26A) and observations (FIG. 26B). Mixture compositions: (i) Cu, (ii) ligand, (iii) Cu-ligand, (iv) p-DOPAmide, (v) p-DOPAmide-ligand, (vi) p-DOPAmide-Cu, (vii) p-DOPAmide-Cu-ligand, (viii) p-DOPAmide-Cu-ligand-coumarin, wherein Cu = CuSO₄, ligand = THPTA, and coumarin = 3-N₃-7-hydroxycoumarin. Arrows indicate liquid/air interface.

FIGS. 27A-27E show investigations of coating topography and thickness, wherein the coating mixture (1 µL) comprises p-DOPAmide, CuSO₄, THPTA, and 3-N₃-7-hydroxycoumarin. FIG. 27A: Schematic of the drop coated surface. FIG. 27B: Fluorescence image of the entire surface at 405 nm excitation after 1 h. FIG. 27C: XPS spectra of Ti 2p at four different surface locations (see FIG. 27A). FIGS. 27D-27E provide AFM images of the coating at locations 2 and 3 (see FIG. 27A).

FIGS. 28A-28C show drop-coating efficiency in terms of density of grafted TAMRA. FIG. 28A provides a schematic overview of grafting strategies by which the surfaces were modified with TAMRA-N₃ (i)-(iii), or TAMRA-NH₂ (iv)-(v): (i) single-step drop coating using a mixture containing p-DOPAmide, Cu, Lig, and TAMRA-N₃; (ii) single-step dip coating using the same mixture in (i); (iii) dip coating with p-DOPAmide, followed by grafting using a mixture of Cu, Lig, TAMRA-N₃, and ascorbate (for reduction of Cu(II) to Cu(I)); (iv) single-step dip coating-grafting with a mixture containing DA and TAMRA-NH₂; (v) dip coating with DA, followed by grafting using TAMRA-NH₂. DA: dopamine, Cu-Lig: CuSO₄-THPTA, Asc: sodium ascorbate. Relative fluorescence emission intensities for Ti/TiO₂ (FIG. 28B) and Si/SiO₂ (FIG. 28C). For each substrate, the lowest intensity was normalized to absorbance unit = 1.0.

FIGS. 29A-29G show single-step multiplexed drop-coating of a US dime with 10 nucleotide long 5′-N₃-DNA-3′-FAM and 3-N₃-7-hydroxycoumarin. FIGS. 29A-29B: Ambient light appearance of a dime before (FIG. 29A) and after (FIG. 29B) drop coating with the MCM containing two MOI-N₃ species, 3-N₃-7-hydroxycoumarin (0.5 mM) and N₃-DNA-FAM (1 µM). FIG. 29C shows a fluorescence image of the coin acquired at 405 nm excitation, indicating the presence of the coumarin. FIG. 29D shows a fluorescence image of the coin acquired at 488 nm excitation, indicating the presence of the DNA. FIG. 29E shows a fluorescence image of the coin acquired with 405/488 nm excitation. FIG. 29F shows a fluorescence image of the drop coated dime with regions (i), (ii), and (iii) indicated. FIG. 29G shows high magnification images highlighting selected regions of the coin with 405, 488, and 405/488 nm excitation.

FIGS. 30A-30G show images of multiplexed drop coating and patterning of surfaces simultaneously grafted with two MOI-N₃ species (3-N₃-7-hydroxycoumarin and N₃-DNA-FAM). FIGS. 30A-30C show fluorescence images of a multiplex patterned cherry tomato at 405 nm (FIG. 30A), 488 nm (FIG. 30B), and 405/488 nm excitation (FIG. 30C). FIGS. 30D-30G show fluorescence images of a multiplex patterned lotus root at 405 nm (FIG. 30D), 488 nm (FIG. 30E), and 405/488 nm excitation (FIG. 30F), as well as an overlay at higher magnification (FIG. 30G).

FIG. 31A shows selective patterning of a plastic polyhedral die (top-down view). FIGS. 31B-31C show selective patterning of a miniature dinosaur toy with the coating (FIG. 31B) and with water droplets added (FIG. 31C).

FIGS. 32A-32B show single-step drop-coating functionalization of 3 dimensional surfaces with coumarin. FIG. 32A shows coating of fruits with uneven smooth surfaces. FIG. 32B shows a coated pin photographed under day light (left) and visualized under 405 nm excitation (right).

FIGS. 33A-33C show patterning various solid surfaces using the described single-step drop coating technology. FIG. 33A shows template-free writing on material surfaces. FIG. 33B shows template-free drawing on material surfaces. Conditions: MOI-N₃ = TAMRA-N₃ (0.5 mM); (i) ambient light images of the mixtures just added on the surfaces; (ii) ambient light images; and (iii) fluorescence images of the resulting surface patterns after 1 h of incubation and subsequent washing. FIG. 33C shows ambient light images of template-free writing and patterning on material surfaces.

FIGS. 34A-34D: Adsorption of FITC-BSA protein on uncoated (FIGS. 34A-34B) and PEG-coated (FIGS. 34C-34D) PP membranes. FIG. 34B and FIG. 34D are cross-sectional images of FIG. 34A and FIG. 34C, respectively. FIG. 34E: Quantification of protein adsorption.

FIGS. 35A-35D show anti-adhesion potencies of PEG coatings against E.coli. FIGS. 35A-35B show colony formation by bacteria detached uncoated (FIG. 35A) and coated (FIG. 35B) samples. FIG. 35C shows live adhered bacteria on uncoated surfaces. FIG. 35D shows dead adhered bacteria on coated surfaces.

FIGS. 36A-36D show anti-adhesion potencies of PEG coatings against S.aureus. FIGS. 36A-36B show colony formation by bacteria detached uncoated (FIG. 36A) and coated (FIG. 36B) samples. FIG. 36C shows live adhered bacteria on uncoated surfaces. FIG. 36D shows dead adhered bacteria on coated surfaces.

FIGS. 37A-37B show S.aureus adhesion on a substrate selectively coated with PEG. The dashed line (FIG. 37B) indicates the coated/uncoated boundary. FIG. 37C shows a 3D view of S.aureus adhesion on a substrate selectively coated with PEG.

FIGS. 38A-38B show anti-biofilm potencies of uncoated (FIG. 38A) and PEG-coated (FIG. 38B) substrates against S.aureus.

FIG. 39 provides quantification of biofilm formation.

FIGS. 40A-40D: Regulating of adhesion and fate of HUVECs on uncoated Ti/TiO₂ surface (FIG. 40A) and Ti/TiO₂ surfaces that were functionalized with BSA (FIG. 40B), c(RGDfK) (FIG. 40C), and PEG (FIG. 40D). Nuclei, vinculin, and cytoskeleton were stained. Inset images provide a lower magnification image showing amounts of adhered cells.

FIGS. 41A-41B: Adhesion of HUVECs on a Ti/TiO₂ surface site-specifically with c(RGDfK) at two different levels of magnification. White dashed line indicates the boundary between coated and uncoated regions (FIG. 41B).

FIG. 42: Cell adhesion of MC3T3-E1 cells on different materials. Higher magnifications (inset) show spreading of adhered cells.

FIGS. 43A-43B: Quantification of (FIG. 43A) adhered cells and (FIG. 43B) average cell spreading area on various materials without (left) and with (right) c(RGDfK) functionalization.

FIG. 44: Cytoskeleton development of MC3T3-E1 cells on Ti/TiO₂ and Si/SiO₂ with nuclei and cytoskeletal staining.

FIG. 45: MTT assay using extracts of uncoated (left) and coated (right) Ti/TiO₂ towards MC3T3-E1 cells.

FIGS. 46A-46B: Cytocompatibility of MC3T3-E1 cells that were treated with extracts of uncoated and c(RGDfK)-coated Ti/TiO₂ at 1 day (FIG. 46A) and 3 days (FIG. 46B). The cells were stained with both Calcein AM and PI.

FIGS. 47A-47B: Adhesion of MC3T3-E1 cells on the struts of porous, 3-dimensional Ti allow scaffolds without (FIG. 47A) and with (FIG. 47B) c(RGDfK) coating for tissue engineering. The cells were stained with both calcein AM and PI.

FIGS. 48A-48B: Modifying 3-dimensional scaffolds and implants for tissue engineering and regeneration. Spatial growth of MC3T3-E1 cells on Ti-based tissue engineering scaffolds without (FIG. 48A) and with (FIG. 48B) c(RGDfK) coating.

FIGS. 48C-48H: Osteogenesis of MC3T3-E1 on a dental implant that was site-specifically coated with c(RGDfK). Bony tissue formation (indicated by asterisks) on the uncoated (FIG. 48C) and coated (FIG. 48D) region after 4 weeks of culturing. Higher magnification imaging shows tissues that were detached from the coated region (FIG. 48E). White arrows indicate mineralized osteoblasts and circles indicate deposited minerals. FIG. 48F provides an SEM image of a mineralized osteoblast attached on the coated implant surface. FIG. 48G provides a higher magnification image of the mineralized osteoblast that shows the mineralized extracellular matrix. FIG. 48H provides an AFM image of extracellular matrix detached from the coated region, showing a composite structure of collage fibers (~50 nm in diameter and extrafibrillar apatite crystals.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt% to about 5 wt% of the material, or about 0 wt% to about 1 wt%, or about 5 wt% or less, or less than, equal to, or greater than about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less. The term “substantially free of” can mean having a trivial amount of, such that a composition is about 0 wt% to about 5 wt% of the material, or about 0 wt% to about 1 wt%, or about 5 wt% or less, or less than, equal to, or greater than about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less, or about 0 wt%.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a nitrogen-containing group including an amine, amide, imine, imide, and a nitrile; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀-₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(=NH)N(R)₂, C(O)N(OR)R, C(=NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(=NH)N(R)₂, C(O)N(OR)R, and C(=NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C═CH, —C≡C(CH₃), —C═C(CH₂CH₃), —CH₂C═CH, —CH₂C═C(CH₃), and —CH₂C═C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “cyanine” as used herein refers to a synthetic dye having an iminium in direct π-conjugation with an enamine, wherein the direct π-conjugation comprises at least one carbon-carbon π-bond (C=C). The N atom of the iminium and/or enamine may comprise a heteroaryl or optionally unsaturated heterocycloalkyl species, or may be optionally substituted with hydrocarbyl substituents. Non-limiting examples of cyanine dye compounds include Cy3, Cy5, Cy3.5, Cy5.5, and Cy7.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise, a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-membered ring with two carbon atoms and three heteroatoms, a 6-membered ring with two carbon atoms and four heteroatoms and so forth. Likewise a C₄-heteroaryl can be a 5-membered ring with one heteroatom, a 6-membered ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3- pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH2, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁—C₄)hydrocarbyl means the hydrocarbyl group can be methyl (C₁), ethyl (C₂), propyl (C₃), or butyl (C₄), and (C₀—C_{b})hydrocarbyl means in certain embodiments there is no hydrocarbyl group.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “drop coating,” as used herein, refers to the action of dropping a liquid onto a surface and takes advantage of surface wetting effects for selective coating.

The term “xeno nucleic acid” as used herein, refers to synthetic nucleic acid analogs having a different sugar backbone than that of the natural nucleic acids (i.e. DNA and RNA). Non-limiting examples of xeno nucleic acids, modifications, and/or derivatives include 2′-fluoro-RNA (2′F-RNA); 2′-O-methyl RNA (2′OMe RNA); LNA (locked nucleic acid); 2′-fluoro-arabinose nucleic acid (FANA); hexitol nucleic acid (HNA); 2′-O-methoxyethyl nucleic acid (2′MOE); (1′-3′-)-β- L-ribonucleic acid (ribuloNA); α-L-threose nucleic acid (TNA); 3′-2′-phosphonomethyl-threosyl nucleic acid (tPhoNA); 2′-deoxyxylonucleic acid (dXNA); phosphorothioate (PS); alkyl phosphonate nucleic acid (phNA); and peptide nucleic acid (PNA).

### Compositions for Surface Functionalization

Provided herein are compositions suitable for functionalizing surfaces. The compositions include a compound of formula (I), or a salt or solvate thereof:

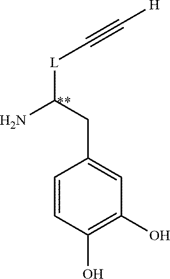

wherein:
L is a linker of formula *-X-(Y)ₘ₁-Z-, wherein * is the bond between X and the carbon marked as **, wherein:
X is a bond (null), —C(═O)—, —C(═O)NH—, —C(═O)N(C_{6—10} aryl)—, —C(═O)N(C_{2—10} alkenyl)—, or —C(═O)N(C_{1—10} alkyl)—, wherein the C₆₋₁₀ aryl is optionally substituted by at least one substituent independently selected from the group consisting of halogen, —R′, —OR′, and —C(═O)OR′;
each occurrence of Y is independently selected from the group consisting of —CH₂CH₂O—, —OCH₂CH₂—, and —CH₂CH₂—, wherein each CH₂ is independently optionally substituted with 1 or 2 CH₃ groups (thus forming —CH(CH3)— or —C(CH3)₂—, respectively);
Z is —(CH2)ₘ₂—, wherein each CH2 is optionally independently substituted with 1 or 2 CH₃ groups (thus forming —CH(CH₃)— or —C(CH3)₂—, respectively);
each occurrence of R′ is independently hydrogen, C₂₋₅ alkenyl, or C₁₋₅ alkyl; ml is 0, 1, 2, 3, 4, 5, 6, 7, 8, 9, or 10;
m2 is 0, 1, 2, 3, 4, and 5;
with the proviso that L is not —C(═O)NHCH₂—.

In certain embodiments, the compound of formula (I) is:

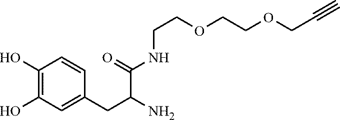 [SMILES: C#CCOCCOCCNC(=O)C(N)Cc1ccc(O)c(O)c1]

2-amino-3-(3,4-dihydroxyphenyl)-N-(2-(2-(prop-2-yn-1-yloxy)ethoxy)ethyl)propanamide, or a salt, solvate, stereoisomer, tautomer, or any mixtures thereof.

In certain embodiments, the compound of formula (I) is:

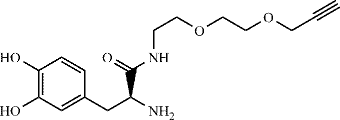 [SMILES: C#CCOCCOCCNC(=O)[C@@H](N)Cc1ccc(O)c(O)c1]

(S)amino-3-(3,4-dihydroxyphenyl)-N-(2-(2-(propyn-1-yloxy)ethoxy)ethyl)propanamide or a salt, solvate, tautomer, or any mixtures thereof.

In certain embodiments, the compound of formula (I) is:

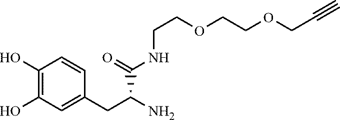 [SMILES: C#CCOCCOCCNC(=O)[C@H](N)Cc1ccc(O)c(O)c1]

(R)amino-3-(3,4-dihydroxyphenyl)-N-(2-(2-(propyn-1-yloxy)ethoxy)ethyl)propanamide or a salt, solvate, tautomer, or any mixtures thereof.

In some embodiments, X is —C(═O)NH— or —C(═O)N(CH₃)—. In other embodiments, at least one Y is —CH₂CH₂O— or —OCH₂CH₂—. In some embodiments, L is —C(═O)NH(CH₂CH₂O)miZ—. In some embodiments, Z is a bond or —CH₂—. In one embodiment, L is —C(═O)NH(CH₂CH₂O)₂— —C(═O)NH(CH₂CH₂O)₂CH₂—, or —C(═O)NH(CH₂CH₂O)CH₂—.

The concentration of the compound of formula (I) in the composition can be about 0.00001 to about 1 M. In some embodiments, the concentration of the compound of formula (I) in the composition is about 0.00001 to about 0.8 M, about 0.0001 to about 0.8 M, about 0.001 to about 0.6 M, or about 0.1 to about 0.4 M. In certain embodiments, the concentration of the compound of formula (I) in the composition is about 0.00001, 0.0001, 0.001, 0.01, 0.05, 0.1, 0.2, 0.3, 0.4, 0.5, 0.6, 0.7, 0.8, 0.9, or about 1 M.

The composition for functionalizing surfaces also includes a copper (II) salt, a copper (I) ligand, and an azido compound (R-N₃). Suitable copper (II) salts include, but are not limited to, copper (II) sulfate, copper (II) chloride, copper (II) bromide, copper (II) iodide, copper(II) perchlorate, copper (II) nitrate, copper (II) hydroxide, hydrates thereof, and mixtures thereof. In certain embodiments the copper (II) salt is copper (II) sulfate or hydrates thereof. The concentration of the copper (II) salt(s) in the composition can be about 0.00001 to about 1 M. In some embodiments, the concentration of copper (II) salt(s) in the composition is about 0.00001 to about 0.8 M. In certain embodiments, the concentration of copper (II) salt(s) in the composition is about 0.0001 to about 0.8 M. In certain embodiments, the concentration of copper (II) salt(s) in the composition is about 0.001 to about 0.6 M. In certain embodiments, the concentration of copper (II) salt(s) in the composition is about 0.1 to about 0.4 M. In certain embodiments, the concentration of copper (II) salt(s) in the composition is about 0.00001, 0.0001, 0.001, 0.01, 0.05, 0.1, 0.2, 0.3, 0.4, 0.5, 0.6, 0.7, 0.8, 0.9, or about 1 M. In one embodiment, the concentration of copper (II) salt(s) in the composition is about 0.00001 to about 0.1 M. In certain embodiments, the concentration of copper (II) salt(s) in the composition is about 0.0001 to about 0.1 M. In certain embodiments, the concentration of copper (II) salt(s) in the composition is about 0.001 to about 0.1 M. In certain embodiments, the concentration of copper (II) salt(s) in the composition is about 0.01 to about 0.1 M.

A “copper (I) ligand” as used herein means a ligand that forms a complex with copper (I) in solution, and/or chelates copper (I). Suitable copper (I) ligands include, but are not limited to, THPTA (tris(3-hydroxypropyltriazolylmethyl)amine), TBTA (tris[(1-benzyl-1H-1,2,3-triazol-4-yl)methyl]amine), BTTES (2-4-(bis-1-tert-butyl-1H-1,2,3-triazol-4yl) methylamino(methyl-1H-1,2,3-triazol-1-yl)ethanesulfonic acid), N¹-(2-(dimethylamino)ethyl)-N′,N′,N′-trimethylethane-1,2-diamine, N¹,N^{1′} -(ethane-1,2-diyl)bis(N¹,N²,N²-trimethylethane-1,2-diamine), 2,2′-bipyridine, and combinations thereof. In certain embodiments, the copper (I) ligand is THPTA. In some embodiments, the copper (I) ligand is present in a catalytic amount. In certain embodiments, the copper (I) ligand is present in a stoichiometric amount. In certain embodiments, the copper (I) ligand is present in excess relative to the amount of the copper (II) salt. The copper (I) ligand can be present in an amount of about 1 mol% to about 400 mol% relative to the amount of copper (II) salt. In some embodiments, the composition contains about 1 to about 20 mol% of copper (I) ligand. In certain embodiments, the composition contains about 1 to about 10 mol% of copper (I) ligand. In certain embodiments, the composition contains about 1 to about 5 mol% of copper (I) ligand. In certain embodiments, the amount of copper (I) ligand is about 1, 1.5, 2, 2.5, 3, 3.5, 4, 4.5, 5, 5.5, 6, 6.5, 7, 7.5, 8, 8.5, 9, ,9.5, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 220, 240, 260, 280, 300, 320, 340, 360, 380, or 400 mol%.

The composition for functionalizing surfaces can also include a reductant. As used herein, the term “reductant” means a substance that slows or inhibits oxidation of copper (I) species in solution but does not otherwise adversely affect the reactivity of the composition herein. Suitable reductants include, but are not limited to, ascorbic acid and hydroquinone. Reductants can be present in a catalytic amount, a stoichiometric amount, or in excess relative to the amount of the copper (II) salt. The reductant can be present in an amount of about 1 mol% to about 400 mol% relative to the amount of copper (II) salt. In some embodiments, the composition contains about 1 to about 20 mol%, about 1 to about 10 mol%, or about 1 to about 5 mol% of reductant. In certain embodiments, the amount of reductant is about 1, 1.5, 2, 2.5, 3, 3.5, 4, 4.5, 5, 5.5, 6, 6.5, 7, 7.5, 8, 8.5, 9, ,9.5, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 220, 240, 260, 280, 300, 320, 340, 360, 380, or 400 mol%.

The composition for functionalizing surfaces can also include an oxidant. As used herein, the term “oxidant” means a substance that increases the rate or accelerates the formation of the coating through the oxidation of compounds of Formula (I), but does not otherwise adversely affect the reactivity of the composition herein. Suitable oxidants include, but are not limited to, sodium periodate, ammonium peroxodisulfate, sodium persulfate, manganese (II) and (III) salts, cerium(IV) ammonium nitrate, iron (III) salts, reactive oxygen species (e.g., hydrogen peroxide, hydroxyl radical, superoxide), peroxidase enzymes (e.g., Horseradish peroxidase), and/or UV irradiation, as well as combinations of these oxidants. Oxidants can be present in a catalytic amount, a stoichiometric amount, or in excess relative to the amount of the copper (II) salt. The oxidant can be present in an amount of about 1 mol% to about 400 mol% relative to the amount of copper (II) salt. In some embodiments, the composition contains about 1 to about 20 mol%, about 1 to about 10 mol%, or about 1 to about 5 mol% of oxidant. In certain embodiments, the amount of oxidant is about 1, 1.5, 2, 2.5, 3, 3.5, 4, 4.5, 5, 5.5, 6, 6.5, 7, 7.5, 8, 8.5, 9, ,9.5, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 220, 240, 260, 280, 300, 320, 340, 360, 380, or 400 mol%.

In various embodiments, the R group in the azido compound (R-N₃) can be any suitable agent of interest. In some embodiments, R can be, without limitation, a chromophore, a fluorogenic molecule, an oligonucleotide, polynucleotide, a nucleic acid, polyethylene glycol, natural polymers, synthetic polymers, a peptide, a polypeptide, a protein, a therapeutic agent, or a lipid, or combinations of the foregoing. In certain embodiments, the chromophore or fluorogenic molecule is covalently linked to an oligonucleotide or polynucleotide. In certain embodiments, the oligonucleotide or polynucleotide comprises between 1 and 10 nucleotides. In certain embodiments, the oligonucleotide or polynucleotide comprises between 10 and 50 nucleotides. In certain embodiments, the oligonucleotide or polynucleotide comprises between 50 and 100 nucleotides. In certain embodiments, the oligonucleotide or polynucleotide comprises between 100 and 500 nucleotides. In certain embodiments, the oligonucleotide or polynucleotide comprises at least two of a deoxyribonucleic acid (DNA), ribonucleic acid (RNA), or xeno nucleic acid (XNA), or any combination thereof. In certain embodiments, the chromophore or fluorogenic molecule is at least one selected from the group consisting of 3′,6′-dihydroxyspiro[isobenzofuran-1(3H),9′-[9H]xanthen]-3-one (Fluorescein), nitrobenzoxadiazole (NBD), 4,4-difluoro-4-bora-3a,4a-diaza-s-indacene (BODIPY), cyanine, rhodamine (RMA), carboxytetramethylrhodamine (TAMRA), or a derivative thereof.

In certain embodiments, R-N₃ is 3-N₃-7-hydroxycoumarin. In certain embodiments, R-N₃ is TAMRA-N₃. In certain embodiments, R-N₃ is 5′-N₃-AGCGTGACTT-3′-Fluorescein (N₃-DNA-FAM). In certain embodiments, R-N₃ is polyethylene glycol-N₃ (PEG-N₃). In certain embodiments, R-N₃ is cyclo[Arg—Gly—Asp—D—Phe—Lys(Azide)] (c(RGDfK)-N₃). In certain embodiments, R-N₃ is Bovine serum albumin with an azide modification (BSA-N₃).

The agent can be a small molecule (MW < about 1000 Daltons) or a large molecule (MW > about 1000 Daltons). For example, the agent can be coumarin or a rhodamine-based fluorogenic molecule (organic dyes), polyethylene glycol (synthetic oligomer), DNA (nucleic acid), cyclic RGD (peptide), and bovine serum albumin (protein). Suitable lipids include, but are not limited to, steroids, fatty acids, and phospholipids. Suitable therapeutic agents include, but are not limited to, small molecule drugs, antibodies, and antibody-drug conjugates. The concentration of R-N₃ in the composition can be about 0.00001 to about 1 M. In some embodiments, the concentration of R-N₃ in the composition is about 0.00001 to about 0.8 M, about 0.0001 to about 0.8 M, about 0.001 to about 0.6 M, or about 0.1 to about 0.4 M.. In certain embodiments, the concentration of R-N₃ in the composition is about 0.00001, 0.0001, 0.001, 0.01, 0.05, 0.1, 0.2, 0.3, 0.4, 0.5, 0.6, 0.7, 0.8, 0.9, or about 1 M. In one embodiment, the concentration of R-N₃ in the composition is about 0.00001 to about 0.1 M, about 0.0001 to about 0.1 M, about 0.001 to about 0.1 M, or about 0.01 to about 0.1 M.

In various embodiments, the composition for functionalizing surfaces comprises an aqueous solution of the compound of formula (I), the copper (II) salt, the copper (I) ligand, and R-N₃. The composition can be in the form of an aqueous buffer, where the pH of the composition is buffered at value of about 5-9. In some embodiments, the composition is buffered at a pH of 5, 5.5, 6, 6.5, 7, 7.5, 8, 8.5, or 9. The buffer can use any suitable buffering agent, depending upon the desired pH. Suitable buffering agents include, but are not limited to, citric acid, KH₂PO₄, NaH₂PO₄, N-cyclohexyl-2-aminoethanesulfonic acid (CHES), borate, TAPS ([Tris(hydroxymethyl)methylamino]propanesulfonic acid), Bicine (2-(Bis(2-hydroxyethyl)amino)acetic acid), Tris (Tris(hydroxymethyl)aminomethane), Tricine (N-[Tris(hydroxymethyl)methyl]glycine), TAPSO (3-[N-Tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (Piperazine-N,N′-bis(2-ethanesulfonic acid)), MES (2-(N-morpholino)ethanesulfonic acid), and the like.

In some embodiments, the composition for functionalizing surfaces does not polymerize at a temperature of about 4° C. or lower. Cooling of the composition is sufficient to substantially inhibit or prevent polymerization, and the composition can be stored at a temperature of about 4° C. or lower without polymerization for a period of up to 6 months.

Compounds of formula (I) or otherwise described herein can be prepared by the general schemes described herein, using the synthetic method known by those skilled in the art. The following examples illustrate non-limiting embodiments of the compound(s) described herein and their preparation.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/ or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form. In certain embodiments, the compound(s) described herein can exist as tautomers.

The compounds described herein, and other related compounds having different substituents are synthesized using techniques and materials described herein and as described, for example, in Fieser & Fieser’s Reagents for Organic Synthesis, Volumes 1-17 (John Wiley and Sons, 1991); Rodd’s Chemistry of Carbon Compounds, Volumes 1-5 and Supplementals (Elsevier Science Publishers, 1989); Organic Reactions, Volumes 1-40 (John Wiley and Sons, 1991), Larock’s Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while coexisting amino groups are blocked with fluoride labile silyl carbamates.

Allyl blocking groups are useful in the presence of acid- and base- protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

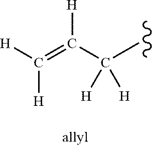

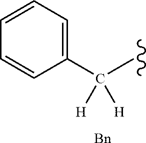

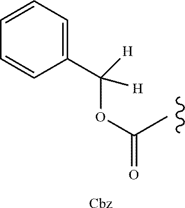

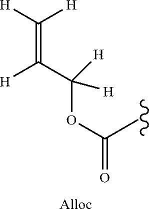

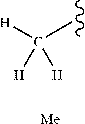

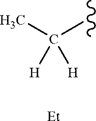

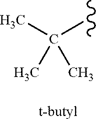

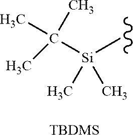

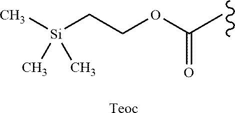

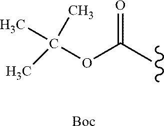

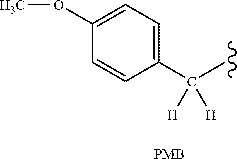

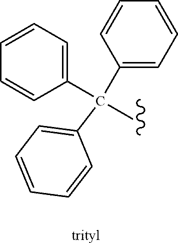

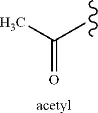

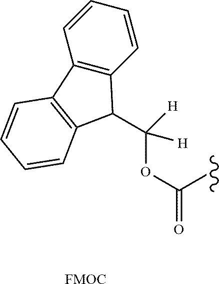

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Methods of Surface Functionalization

Provided herein is a method of coating a surface by contacting at least a portion of the surface with a composition for functionalizing surfaces that includes a compound of formula (I), a copper (II) salt, a copper (I) ligand, and an azido compound (R—N₃), wherein R comprises a chromophore, fluorogenic molecule, oligonucleotide, synthetic homopolymer (e.g., polyethylene glycol), block copolymer (e.g., polyethylene glycol-polycaprolactone or polyethylene glycol-polylactide), nucleic acid, polyethylene glycol, peptide, polypeptide, protein, therapeutic agent, or lipid, and wherein at least a portion of the surface is coated with a reaction product of the compound of formula (I) and the azido compound. The composition can further include at least one an oxidant and/or a reductant. In one embodiment, the method is performed in a single step, whereby after an initial coating with the compositions described here, no further coating is necessary. Coated surfaces can be used in a variety of applications, including without limitation, semiconductor, biological (e.g. medical and/or dental implants), electronic, catalyst, and coating (e.g., paint, sealer, etc.) applications.

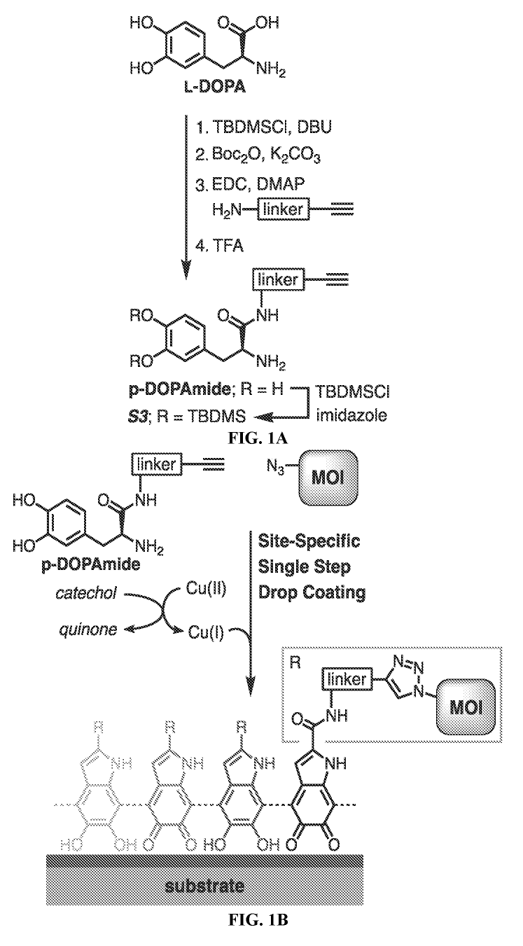

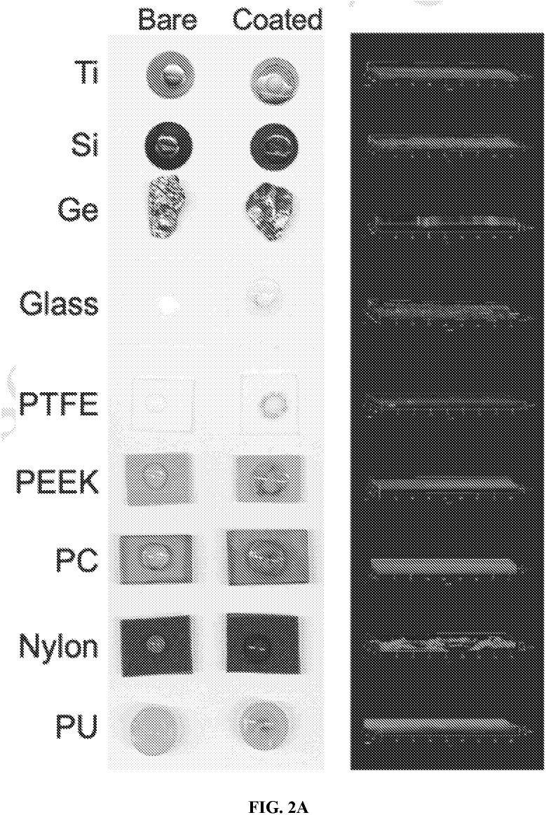

In various embodiments, the compound of formula (I), copper (II) salt, copper (I) ligand, and azido compound are part of an aqueous mixture when contacted with the surface. In some embodiments the compound of formula (I) is added to an aqueous mixture of copper (II) salt, copper (I) ligand, and R-N₃ and subsequently deposited on a surface. The aqueous mixture can be a solution. The acetylene group in the compound of formula (I) can react with the azido group in R—N₃ in the composition for functionalizing surfaces, as shown in FIG. 1, to form a triazole, thereby covalently linking the R group in the azido compound to the compound of formula (I). The catecholamine moiety (3,4-dihydroxyphenylalanine) can, in some embodiments, oxidatively autopolymerize to form an adhesive coating on the surface.

In various embodiments, the composition is applied to the surface by drop coating, whereby by a drop or small amount of the composition is deposited (dropped) onto the surface of a substrate. The resulting polymer self-assembles on the substrate surface to form a stable coating within about 20 to about 30 minutes. The droplet volume, concentration of compound of formula (I) and R—N₃, and reaction time can be varied to modify the thickness of the coating and density of grafting. Multipurpose functionalization of the surface can be achieved via micropatterning, multi-component grafting, or layer-by-layer coating. The method provides, for example, 1) the ability to 1) create micro-scale coated regions (e.g., with diameter <0.2 mm); 2) coat using microvolumes (e.g., <0.2 µL); 3) promote material-independent adhesion; 4) coat 3-dimensional surfaces; and 5) provide tailorable grafting density. In some embodiments, the composition can be applied to a surface using dip coating, whereby the substrate is dipped into the composition described herein. In some embodiments, the composition can be applied to a surface using spin coating, whereby the substrate is spun at particular rate after an amount of the composition is deposited on the substrate surface. In some embodiments, multiple layers can be deposited onto the surface, and each layer can have the same or different R group. Thus, for example, a surface can be coated with a fluorogenic molecule, such as 3-azido-7-hydroxycoumarin and a nucleic acid.

In various embodiments, the surface includes metal, stone, glass, wood, ceramic, semi-conductor, polymer, inorganic material, or combinations thereof. The surface can be untreated or pretreated. Pretreated surfaces include, without limitation, surfaces that have been treated with chemicals, flame, plasma, UV light, ozone, and the like, or combinations of such treatments.

Suitable semi-conductor materials include, but are not limited to, germanium, silicon dioxide, titanium oxide, gallium arsenide, graphene, gallium nitride, and the like. Suitable inorganic materials include, without limitation, metal oxides, minerals, nanotubes, and the like. Metals include both pure metals (elements) and alloys of metals. Suitable polymers include, but are not limited to, polytetrafluoroethylene, polyether ether ketone, polycarbonate, low-density polyethylene, high-density polyethylene, polypropylene, polystyrene, polyvinyl chloride, silicon rubber, polychlorotrifluoroethylene, nylon, polysiloxane, polyethylene terephthalate, polyacrylate, polyacrylamide, polyester, polycarbonate, polyurethane, or combinations thereof.

### Materials and Methods

### Reagents

3,4-Dihydroxy-L-phenylalanine (L-DOPA) was purchased from Alfa Aesar. t-Butyldimethylsilyl chloride (TBDMSC1), 1,8-diazabicyclo-[5.4.0]-undec-7-ene (DBU), di-tert-butyl decarbonate (Boc₂O), potassium carbonate (K₂CO₃), 3-(ethylimino-methylideneamino)-N,N-dimethylpropan-1-amine (EDC), 4-(dimethylamino)-pyridine (DMAP), trifluoroacetic acid (TFA), imidazole, p-toluenesulfonyl chloride (p-TsCl), triethylamine (Et₃N), sodium azide (NaN₃), copper(II) sulfate (CuSO4), sodium ascorbate, dopamine (DA) (available in hydrochloride salt form), hydrogen peroxide, paraformaldehyde, iron(III) chloride, ethylenediaminetetraacetic acid (EDTA), glutaraldehyde, α-MEM medium, bovine serum albumin (BSA), anti-vinculin monoclonal antibody, fluorescein isothiocyanate (FITsC)-labeled goat anti-rabbit IgG, tetramethylrhodamine-isothiocyanate (TRITC)-phalloidin kit, 4′,6-diamidino-2-phenylindole dihydrochloride (DAPI), actin cytoskeleton/focal adhesion staining kit (FAK100), and Triton X-100 were purchased from Millipore-Sigma. Tris(3-hydroxypropyltriazolyl-methyl)amine (THPTA) was purchased from Click Chemistry Tools. 2-[2-(2-Propynyloxy)ethoxy]ethylamine was purchased from TCI America. Micro bicinchoninic acid (MicroBCA) was purchased from Thermo Scientific. LIVE/DEAD® Baclight™ Bacterial Viability Kit was purchased from Molecular Probes, Inc., Invitrogen. Calcein AM and propidium iodide (PI) were purchased from Dojindo, Japan. 3-(4,5-dimethylthiazol-2-yl)-2,5-diphenyl tetrazolium bromide (MTT) cell proliferation assay kit was purchased from BioVision Inc. TAMRA-NH₂ was purchased from Adipogen Life Sciences.

### General Synthetic Methods

For the chemical synthesis of organic compounds, all reactions were performed under a dry nitrogen atmosphere unless otherwise stated. All glassware was oven-dried before use. Purification of the synthesized compounds was performed using a Büchi Reveleris® flash chromatography system equipped with a FlashPure EcoFlex silica (50 µm sphere) column. Observed rotation (a_{obs}) values were measured in a standard glass cell (100 mm, 1 mL) using a sodium D-line lamp at 20° C. in a PerkinElmer Model 241 Polarimeter. Specific rotation [a] was calculated based on [a]²⁰_{D} = (a_{obs})/[(gₛₐₘₚₗₑ in 1 mL) × 1 dm]. Nuclear Magnetic Resonance (NMR) spectroscopic analyses were carried out on either a Varian VNMRS 500 MHz or Bruker Avance Neo 500 MHz spectrometer. NMR data is provided for new compounds. ¹H NMR spectra were acquired at 500 MHz and ¹³C NMR spectra were acquired at 125 MHz. Chemical shifts (δ) for 1 H NMR spectra were referenced to (CH₃)₄Si at δ = 0.00 ppm, to CD₂HCN at δ = 1.94 ppm, to CHD₂S(O)CD₃ at δ = 2.50 ppm, or to CHCl₃ at δ = 7.27 ppm. ¹³ C NMR spectra were referenced to CD₃S(O)CD₃ at δ = 39.51 ppm, to CDC13 at δ = 77.23 ppm, or to CD₃CN at δ = 118.70 ppm. The following abbreviations are used to describe NMR resonances: s (singlet), d (doublet), t (triplet), m (multiplet), br (broad), and nfom (non-first order multiplet). Coupling constants (J) are reported in Hz. Low-resolution mass spectroscopy (LRMS) analysis was performed using a Finnigan LCQ™ DUO mass spectrometer. Liquid chromatography followed by high-resolution mass spectroscopy (LC-HRMS) analysis in the ESI mode was carried out on a Waters Acquity-Xevo G2-XS QTof.

### MOI-N₃ Molecules

3-N₃hydroxycoumarin, TAMRA-N₃, 5′-N3-AGCGTGACTT-3′-Fluorescein (N₃-DNA-FAM), Polyethylene glycol-N₃ (PEG—N₃), Cyclo[Arg—Gly—Asp—D—Phe—Lys(Azide)] (c(RGDfK)-N₃), and Bovine serum albumin with an azide modification (BSA—N₃). 3-N₃-7-hydroxycoumarin was purchased from Combi-Blocks. TAMRA-N₃ was purchased from Adipogen Life Sciences. N₃-DNA-FAM was purchased from Integrated Device Technology, Inc. PEG-N3 was synthesized from PEG methyl ether (MW~750 g/mol), which was purchased from BeanTown Chemical. c(RGDfK)—N₃ was purchased from Peptides International. BSA-N₃ was purchased from ProteinMods.

### Buffers and Solvents

Phosphate-buffered saline (PBS), 4-morpholineethanesulfonic acid (MES), and tris(hydroxymethyl)aminomethane (Tris) were purchased from Millipore-Sigma. Acetonitrile (CH₃CN), dichloromethane (CH₂Cl₂), dioxane, dimethylformamide (DMF), ethanol (EtOH), and tetrahydrofuran (THF) were purchased from Millipore Sigma. Methanol (MeOH), diethyl ether (Et₂O), and ethyl acetate (EtOAc) were purchased from Fisher Scientific. Deuterated solvents were purchased from either Cambridge Isotope Laboratories or Millipore Sigma. Deuterated solvents contained 0.05% (v/v) TMS as a secondary internal reference. Water was deionized and filtered to a resistivity of 18.2 ΩM with a Milli-Q® Plus water purification system (Millipore, Massachusetts). Buffers were prepared freshly in Milli-Q® water and their pH were adjusted using HCl or NaOH. For all experiments, the buffer concentration was 10 mM unless otherwise stated.

### Materials

Poly(tetrafluoroethylene) (PTFE), poly(ether ether ketone) (PEEK), nylon, polycarbonate (PC), and silicone rubber (SiR) were purchased from McMaster-Carr. Glass was purchased from VWR International. Si/SiO2 wafer substrate was purchased from University Wafer. All of these materials were cleaned ultrasonically in ethanol and water for 15 min before use. Commercially available pure titanium rods were cut into plates and polished up to 1200 grit using SiC paper. Ti-based materials were subjected to successive ultrasonic rinses in acetone, ethanol, and water for 15 min before use. Germanium (Ge) was purchased from Millipore-Sigma and used as received. Polypropylene (PP) membrane was purchased from Deschem Science Supply, China. Macroporous Ti-6Al-4V scaffolds were acquired from AKEC Medical Co. Ltd, China. Dental implant (ProActive ∅5.0 × 9 mm) was purchased from Neoss Inc.

### Biologics

Bacteria culture: Staphylococcusaureus (S.aureus, ATCC-6538) and Escherichiacoli (E.coli, ATCC-25922) were purchased from American Type Culture Collection (ATCC). Mammalian cell culture: Human Umbilical Vein Endothelial Cells (HUVEC) were purchased from PromoCell, Germany. Mouse pre-osteoblast cell line MC3T3-E1 subclone 14 cells was purchased from ATCC (CRL-2594).

### General Single-Step Drop Coating Procedure

Solutions of CuSO₄ (5 mM), THPTA (10 mM), MOI-N₃ (1 µM - 0.5 mM), and p-DOPAmide (10-50 mM) were prepared using one of the following buffers: MES (pH 5.5), PBS (pH 7.4), or Tris (pH 8.5). These solutions were combined in an Eppendorf vial to provide a master coating mixture (MCM). Unless otherwise specified, all the buffers and reagent solutions were thoroughly bubbled with N₂ gas (for ~15 min) to remove molecular oxygen from the liquid. A specified volume of the MCM was dropped onto a material in a tissue culture polystyrene plate (TCPS) or Petri dish, which was then sealed with Parafilm M (Bemis) and gently agitated on a shaker at 37° C. After the coating is complete (typically within 30 min - 4 h unless otherwise stated), the substrate was rinsed thoroughly with Milli-Q water and dried under air and at RT.

### Material Substrates

Planar solid materials used in this study include Ti/TiO₂, Si/SiO₂, glass, polytetrafluoroethylene (PTFE), polyether ether ketone (PEEK), polycarbonate (PC), silicone rubber (SiR), and a dime coin. 2-dimensional porous or fibrous materials include nylon foam and polypropylene (PP) membrane. 3-dimensional objects include germanium pieces, a plastic polyhedral dice, mini dinosaur toy, cherry tomato (hydrophobic), lotus root (porous and hydrophilic), porous Ti-based tissue scaffold, and Ti-based dental implant.

### Surface Characterization Techniques

AFM was carried out in flapping mode for surface topography and roughness using a Cypher microscope (Asylum Research) and standard SiN cantilevers (AC160, Asylum). Additionally, the coating thickness was determined as height differences between the coating and a scratched area. SEM (Zeiss Sigma, German) was performed at an accelerating voltage of 2 keV under vacuum. TEM (JEOL JEM 2010F, Japan) was operated at 200 keV for microstructural images with aid of a digital camera. Fourier transform infrared spectroscopy (FTIR) was used to probe surface functional groups on a PerkinElmer Spectrum 100 Spectrometer (PerkinElmer) equipped with an attenuated total reflectance (ATR) accessory. Spectra were acquired in the range of 580-4000 cm⁻¹ with 32 scans for each spectrum at a resolution of 1 cm⁻¹. Micro-Raman spectra were recorded on a Raman microscope (Renishaw inVia, Ar⁺ 532 nm, UK). For element compositions, XPS (K-Alpha™, Thermo Scientific) investigations were conducted using monochromatic Al Kα source (hv = 1486.6 eV) at an energy step of 0.05 eV (core-level spectra) or 0.5 eV (survey spectra). CLSM (Zeiss LSM780) was employed for fluorescence imaging under various scanning modes: XY, Z-stacks, and tile scans as if necessary. For Z-stacks, a series of sliced images were acquired and later reconstructed into a 3-dimentional field of view. For tile scans, a motorized stage was driven by the ZEN software (Zeiss) to capture multi-field images across the XY plane, which were merged into a full-scale image. Images were processed and analyzed using either ZEN or ImageJ.

### Surface Wettability

Static contact angles (CA) were measured at room temperature (RT) using the sessile drop method on a custom-built benchtop CA goniometer (L2004A1, Ossila) equipped with a video camera. Each time, 5-µL aliquots of Milli-Q water were added to the air side of sample surface and images were recorded after droplet spreading.

### Material-Independent Drop-Coating

A mixture of CuSO₄, THPTA, and 3-azido-7-hydroxycoumarin was prepared using 10 mM PBS buffer (pH 7.4). This solution was combined with the compound of formula (I) in a 2.0-mL vial to give final concentrations of 5 mM of CuSO₄, 10 mM of THPTA, 0.5 mM of 3-azido-7-hydroxycoumarin, and 50 mM of the compound of formula (I). A characteristic brown color was observed within 3 seconds after mixing all the reagents described above. For a typical drop-coating protocol, a 25-µL aliquot of the final reagent mixture was dropped onto a material surface in a Petri dish, which was then sealed with Parafilm M (Bemis) and gently agitated on a shaker at 37° C. for 4 h. After the coating is complete, the substrate was rinsed thoroughly with MilliQ water and dried under ambient condition (room temperature and pressure).

### Surface Functionalization With DNA

A mixture of CuSO₄, THPTA, and a 10-nt long 5′-N3-DNA-3′-FAM was prepared using 10 mM PBS buffer (pH 7.4). This solution was combined with the compound of formula (I) in a 2.0-mL vial to give final concentrations of 5 mM of CuSO₄, 10 mM of THPTA, 0.0001 mM of the DNA, and 10 mM of the compound of formula (I). 100-µL droplets of the resulting mixture were dropped onto titanium or silicon substrate, which was then incubated at 37° C. for 4 h.

### Patterning of 2- or 3-Dimensional Objects

1-2 µL droplets of the MCM (MOI-N₃ = 3-N₃-7-hydroxycoumarin, 0.5 mM) were added onto irregular surfaces of 3-dimensional objects (a plastic polyhedral dice and mini dinosaur toy), which were then incubated for 1 h.

### Multiplexed Patterning

A mixture of CuSO₄, THPTA, 3-azido-7-hydroxycoumarin, and a 10-nt long 5′-N3-DNA-3′-FAM was prepared using 10 mM PBS buffer (pH 7.4). This solution was combined with the compound of formula (I) in a 2.0-mL vial to give final concentrations of 5 mM of CuSO₄, 10 mM of THPTA, 0.5 mM of 3-azido-7-hydroxycoumarin, 0.0001 mM of the DNA, and 50 mM of the compound of formula (I). 400 µL of the MCM containing two MOI-N₃ (3-N₃-7-hydroxycoumarin and N₃-DNA-FAM) was dropped onto a dime coin, which was then incubated for 4 h.

In another example, 1-2 µL droplets of this MCM were added onto a cherry tomato and lotus root, which were both then incubated for 1 h. Other material-independent patterns were generated according to the following protocol: 1-5 µL droplets of the MCM (MOI-N₃ = 3-N₃-7-hydroxycoumarin or TAMRA-N₃, either 0.5 mM), were added onto substrates (Ti/TiO₂, glass, Si/SiO₂, and PEEK) to create micro-volume arrays or patterns.

### Material-Independent Patterning

A mixture of CuSO₄, THPTA, and 3-azido-7-hydroxycoumarin was prepared using 10 mM phosphate-buffered saline (PBS) buffer (pH 7.4). This solution was combined with the compound of formula (I) in a 2.0-mL vial to give final concentrations of 5 mM of CuSO₄, 10 mM of THPTA, 0.5 mM of 3-azido-7-hydroxycoumarin, and 50 mM of the compound of formula (I). 1-µL droplets of the resulting mixture were added onto surfaces of different materials by dropping or plotting along a designated pathway. Micro-volume droplet arrays and patterns were successfully created on titanium, silicon, PTFE, PEEK, and Nylon.

### p-DOPAmide Concentration and Reaction Time Studies

Ti/TiO₂ was drop-coated for 1-12 h using 100-µL aliquots of the MCM (MOI-N₃ = 3-N₃-7-hydroxycoumarin, 0.5 mM; p-DOPAmide, 1-50 mM). Samples were taken, rinsed, and examined by X-ray photoelectron spectroscopy (XPS) and CLSM at 405 nm excitation. Concomitantly, the time-dependent evolution of various coating mixtures was monitored in small Eppendorf vials.

### Additive Studies

Substrates were drop coated with 100-µL droplets of the MCM (MOI-N₃ = 3-N₃-7-hydroxycoumarin, 0.5 mM) that was supplemented with FeCl₃ (5 or 0.5 mM). Control substrates were drop coated without FeCl3. After 4 h of incubation, each coated substrate was investigated by ambient light photography, XPS, and CLSM.

### Catechol Protection Studies

O-TBDMS-protected p-DOPAmide (compound S3) was used in this investigation. A mixture of CuSO₄ (5 mM), THPTA (10 mM), 3-N₃-7-hydroxycoumarin (0.5 mM), and S3 (10 mM) was dropped onto Ti/TiO₂ substrates. Control substrates were drop coated with the MCM, which contained p-DOPAmide instead of S3. After 4 h of incubation, each substrate was investigated by ambient light photography, XPS, and CLSM.

### Grafting Efficiency Based on Grafting Density Studies

Ti/TiO₂ and Si/SiO₂ were functionalized with TAMRA via five different methods (i: drop coating; ii-v: dip coating):
(i) Single-step drop coating with p-DOPAmide. An MCM (MOI-N₃ = TAMRA-N₃, 0.5 mM) was prepared using a PBS buffer (pH 7.4). A 100-µL droplet of this MCM was dropped onto the substrate, which was then incubated at 37° C. for 6 h.
(ii) Single-step dip coating with p-DOPAmide. 500-µL of the same MCM was added into a 24-well TCPS containing the substrate, which was then incubated at 37° C. for 6 h.
(iii) Stepwise dip coating with p-DOPAmide. Substrates were immersed in 10 mM of p-DOPAmide in Tris buffer (pH 8.5) at RT for 3 d. The resulting substrates were then incubated at 37° C. for 6 h with a N₂-bubbled mixture of CuSO₄ (5 mM), THPTA (10 mM), TAMRA-N₃ (0.5 mM), and sodium ascorbate (25 mM), which was buffered with PBS (pH 7.4).
(iv) Single-step dip coating with DA. A mixture of DA (10 mM) and TAMRA-NH₂ (0.5 mM) was prepared using Tris buffer (pH 8.5). Substrates were immersed into this mixture and incubated at 37° C. for 24 h.
(v) Stepwise dip coating with DA. Substrates were immersed into a solution of DA (10 mM) buffered with Tris (pH 8.5) and incubated at 37° C. for 24 h. The resulting substrates were then incubated with TAMRA-NH₂ (0.5 mM) at 37° C. for 6 h.

The resulting substrates were rinsed with Milli-Q water, dried under air at RT, and visualized by confocal laser scanning microscopy (CLSM). Images of each triplicate samples were analyzed by ImageJ to quantify the averaged fluorescence intensities. For each substrate, the lowest intensity was normalized to au of 1.0. Accordingly, the relative fluorescence intensities using methods (i)-(v) were found as: For Ti/TiO₂, (i): 7.0, (ii): 3.5, (iii): 6.4, (iv): 1.1, and (v): 1.0. For Si/SiO₂, (i): 7.9, (ii): 3.4, (iii): 3.7, (iv): 1.2, and (v): 1.0.

### Cu(II)-Ligand Assisted p-DOPAmide Oxidative Polymerization in Solution

Solutions of (i) p-DOPAmide, (ii) p-DOPAmide + Lig, (iii) p-DOPAmide + Cu, and (iv) p-DOPAmide + Cu + Lig were prepared using a buffer (MES pH 5.5, PBS pH 7.4, or Tris pH 8.5) with or without N₂ bubbling. For all solutions, the concentrations of p-DOPAmide, THPTA, and CuSO₄ were 10 mM, 10 mM, and 5 mM, respectively. Solutions that were not bubbled with N₂ were maintained in open Eppendorf vials at RT for 4 h. Subsequently, these vials were loosely covered with a lid to avoid evaporation. Solutions that were bubbled with N₂ (vigorously for 15 min) were transferred into Eppendorf vials, which were tightly sealed with a lid. Lig: Ligand and Cu: CuSO_{4.}

### Catechol-Assisted Cu(I) Production From Cu(II)

Cu(I) production was evaluated using a modified MicroBCA assay. This assay relies on the reduction of Cu(II) ions into Cu(I) ions by proteins (testing samples) in an alkaline buffer (kit component A), which complex with bicinchoninic acid (BCA, kit component B) to give a characteristic violet color. In fact, similar coloration could be generated by using any reagent (in addition to proteins) that has the capability to reduce Cu(II) into Cu(I). The following testing solutions were freshly prepared using PBS: (i) CuSO₄ (5 mM); (ii) THPTA (10 mM); (iii) CuSO₄ (5 mM) and THPTA (10 mM); (iv) p-DOPAmide (10 mM); (v) THPTA (10 mM) and p-DOPAmide (10 mM); (vi) CuSO₄ (5 mM) and p-DOPAmide (10 mM); (vii) CuSO₄ (5 mM), THPTA (10 mM), and p-DOPAmide (10 mM); (viii) CuSO₄ (5 mM), THPTA (10 mM), and S3 (10 mM). These solutions were mixed with MicroBCA kit components A and B at a ratio of 1:5:5. The resulting mixtures were shaken at 37° C. for 30 min to facilitate coloration, and the absorbance was measured at 570 nm.

### Interfacial Film Formation in Drop Coating

Material-independent interfacial film formation. The MCM (MOI-N₃ = 3-N₃-7-hydroxycoumarin, 0.5 mM) was used to coat substrates of these materials: Ti/TiO₂, Si/SiO₂, Ge, glass, PTFE, PEEK, PC, nylon, SiR, and quartz cells. Film debris was collected by washing the substrates and characterized by atomic force microscopy (AFM), transmission electron microscopy (TEM), Raman spectroscopy, and CLSM.

### Surface Functionalization for Antifouling

A mixture of CuSO₄, THPTA, and PEG-N₃, was prepared using 10 mM PBS buffer (pH 7.4). This solution was combined with the compound of formula (I) in a 2.0-mL vial to give final concentrations of 5 mM of CuSO₄, 10 mM of THPTA, and 10 mM of the compound of formula (I). The final concentration of PEG-N₃ was 0.5 (v/v)%. 200-µL droplets of the MCM (MOI-N₃ = PEG-N₃, 0.5 % v/v) were dropped onto PP membranes. The membrane was then incubated at 37° C. for 4 h. To investigate the antifouling properties of the PP surfaces, both uncoated and PEG-functionalized PP membranes were incubated with FITC-BSA (100 µg/mL) at 37° C. for 2 h. The resulting membranes were thoroughly rinsed with PBS buffer. The surface-retained proteins were fixed by 4 (v/v) % paraformaldehyde for 10 min. The final samples were examined by CLSM at 488 nm excitation.

### Surface Functionalization for Antibacterial/Antibiofilm Studies

100-µL droplets of the same MCM mixture were added onto Ti/TiO₂ substrates, which were then incubated at 37° C. for 4 h. Prior to antibacterial tests, all specimens were sterilized in 75% (v/v) EtOH for 20 min and rinsed three times with a sterile PBS buffer. Bacteria culture: S.aureus (ATCC6538) and E.coli (ATCC 25922) strains were cultured using Luria-Bertani (LB) broth or LB agar plates at 37° C. In brief, bacterial cells were shaken (180 rpm) overnight in LB broth and then sub-cultured to a concentration of ~2×10⁸ CFU/mL. The resulting suspensions were diluted to desired concentrations (1.0×10⁵ CFU/mL) for further tests. Antiadhesion assays: 1.0×10⁵ CFU/mL of S. aureus and E. coli strains were inoculated on uncoated and PEG-functionalized Ti/TiO₂ and cultivated for 1 h at 37° C. to allow attachment. To detach surface-adhered bacteria, samples were rinsed gently with PBS and transferred to a sterile Eppendorf tube with 1 mL of fresh LB broth and then sonicated for 10 min. After ten-fold serial dilutions, the suspensions were spread onto LB agar plates and grew overnight to foster colony formation. The LIVE/DEAD® Baclight™ kit was adopted to stain and in situ visualize surface-adhered bacteria under CLSM. Briefly, 400 µL of SYTO (6 µM) and propidium iodide (30 µM) stain mixtures were added to each specimen and maintained for 15 min in darkness. Antibiofilm assays: S. aureus (1.0×10⁵ CFU/mL) was inoculated and cultivated for 5 d at 37° C. Thereafter, samples were rinsed gently with PBS to remove loosely adherent species, followed by LIVE/DEAD staining. CLSM imaging was performed to visualize the bacteria within biofilms. Alternatively, the biomass was quantified using a crystal violet staining method. Samples were fixed in 4% PFA and stained with 0.1% (w/v) crystal violet for 15 min, and washed with PBS buffer gently to remove excess reagents. The stained sample was dissolved in 95% (v/v) EtOH, and absorbance of the solution was measured at 570 nm.

### Surface Functionalization for Mammalian Cell Studies

The MCM mixture ((MOI-N₃ = BSA-N₃ or c(RGDfK)-N₃, either 20 µg/mL, or PEG-N₃, 0.1 % (v/v); CuSO₄ (0.5 mM), THPTA (1 mM), and p-DOPAmide (1 mM)) was dropped onto a material substrate, which was then incubated at 37° C. for 4 h. Specimens were sterilized in 75% (v/v) EtOH for 20 min and rinsed three times with sterile PBS prior to cell studies.

### Cell Cultures

HUVECs were cultured in Endothelial Cell Growth Medium 2 supplemented with Supplement Mix (PromoCell). Pre-osteoblastic MC3T3-E1 cells were cultured in alpha Minimum Essential Medium (α-MEM, Sigma) supplemented with 1% penicillin-streptomycin (hereafter termed as 1% pen-strep, VWR International), and 10% (v/v) fetal bovine serum (FBS, Hyclone Laboratories Inc.) as the growth medium. For osteogenic differentiation, α-MEM that contain the following was used: 1% pen-strep, 10% FBS, 50 µg/mL of ascorbic acid, 10 mM of β-glycerol phosphate, and 100 nM of dexamethasone as the osteogenic medium. Cultures were maintained at 37° C. in a humidified 5% CO₂ atmosphere. Medium was refreshed every 2-3 d. Sub-confluent cells were harvested using 0.05% trypsin-EDTA, collected by centrifugation, and then resuspended to a desired density prior to seeding.

### MOI-Regulated Adhesion of HUVECs

HUVECs (5×10⁴ cells/mL) were seeded onto a drop coated Ti/TiO₂ substrate in 24-well TCPS plates and cultured at 37° C. for 12 h. After the culturing, the cells were stained with a FAK100 kit per manufacturer instructions: Cells were fixed in 4% paraformaldehyde for 10 min, permeabilized in 0.1% Triton X-100 for 2 min, and blocked with 1% BSA/PBS for 30 min. The resulting cells were incubated with an anti-vinculin monoclonal antibody (1:500 dilution) at RT for 1 h, and stained with the following dyes: FITC-conjugated goat anti-mouse IgG (1:100 dilution; 1 h), TRITC-conjugated phalloidin (1:500 dilution; 1 h), and DAPI (1:1000 dilution; 5 min). After thorough rinses, CLSM images of the samples were recorded in a multitrack mode, wherein actin cytoskeleton (via TRITC-phalloidin), focal adhesion (via anti-vinculin), and nuclei (via DAPI) were visualized as red, green, and blue, respectively.

### Site-Selective Adhesion of HUVECs

Ti/TiO₂ surface was drop coated with 1 µL of coating mixture containing c(RGDfK)-N₃. This partially functionalized surface was seeded with HUVECs (1×10⁵ cells/mL) and incubated at 37° C. for 24 h. Thereafter, the cells were stained with 2 µM of Calcein AM for 10 min and investigated by CLSM.

### Site-Selective Adhesion of MC3T3-E1 Cells

Ti/TiO₂, Si/SiO₂, PEEK, and PTFE substrates were drop coated with 100-400 µL of c(RGDfK)-N₃. The uncoated and coated materials were incubated with MC3T3-E1 cells (2×10⁴ cells/mL) at 37° C. for 4 h. Cell adhesion and survival were assessed by staining with Calcein AM (2 µM) and PI (4 µM) for 15 min. Cells were imaged by CLSM, wherein living and dead cells were colored as green and red, respectively. In addition, at 24 h, cytoskeletons for selected cultures were stained with TRITC-conjugated phalloidin and imaged by CLSM.

### Cytotoxicity Assay

Ti/TiO₂ was drop coated with c(RGDfK)-N₃ as described above. The uncoated and coated specimens were each immersed in serum-free α-MEM (1.25 cm²/mL) at 37° C. for 72 h. The leaching fluids (extracts) were collected and supplemented with 10% (v/v) FBS prior to use. Cytotoxicity was evaluated by using an MTT assay according to manufacturer’s instructions. MC3T3-E1 cells (5×10⁴ cells/mL) were seeded in 96-well TCPS plates and incubated for 24 h to allow attachment. Afterwards, the medium was replaced with 100 µL of extracts. After day-1 and day-3, the medium was discarded, and 100 µL of serum-free α-MEM containing 50% (v/v) MTT solution was added to each well. The plates were incubated at 37° C. for 3 h to yield formazan crystals. The formazan was solubilized in an MTT solvent, and its absorbance was measured on a microplate reader (Molecular Devices, SpectraMax iD3) at 590 nm. Alternatively, MC3T3-E1 cells (5×10⁴ cells/mL) were seeded onto a glass surface and incubated for 24 h. The cells were then treated with material extracts as described above. The cells were stained with calcein AM (2 µM) and propidium iodide (4 µM) and investigated by CLSM.

### Tissue Engineering

Macroporous Ti-6A1-4V scaffolds were dip coated with 1 mL of the MCM (MOI-N₃ = c(RGDfK)-N₃). MC3T3-E1 cells were seeded at a density of 2×10⁵ cells/mL and incubated at 37° C. After 4 d, cell adhesion was evaluated by staining with Calcein AM (2 µM) and PI (4 µM) for fluorescence imaging. After 7 d, cytoskeleton development was evaluated by staining with TRITC-Phalloidin for fluorescence imaging.

### In Vitro Osteogenesis on Dental Implant

A commercially available dental implant was partially coated with c(RGDfK)-N₃ as described above. MC3T3-E1 cells (2×10⁵ cells/mL) were seeded and the implant was incubated at 37° C. in a growth medium for 7 d. Afterwards, the medium was replaced by osteogenic medium and cultivation was prolonged up to 28 d. The sample was rinsed with PBS thrice and fixed in 2.5% (v/v) glutaraldehyde in PBS for 2 h, and then dehydrated in a gradient of ethanol (50%-100% v/v) for 15 min each. The sample was dried in air and investigated by scanning electron microscopy (SEM). Alternatively, bony tissues were partially detached, and AFM was used to investigate the topography of extracellular matrix.

### EXAMPLES

Various embodiments of the present application can be better understood by reference to the following Examples which are offered by way of illustration. The scope of the present application is not limited to the Examples given herein.

Example 1: Synthesis of p-DOPAmide

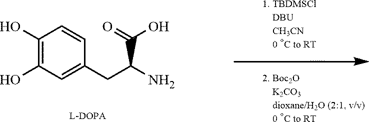

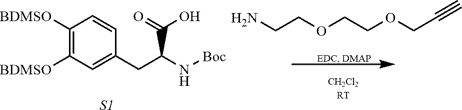

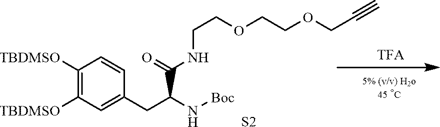

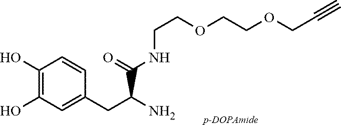

Scheme 1. Synthesis of p-DOPAmide

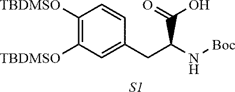

Synthesis of (S)-3-(3,4-bis((tert-butyldimethylsilyl)oxy)phenyl)-2-((tert-butoxycarbonyl)-amino)-propanoic acid (S1)

(Step 1) To a 100-mL round-bottom flask was added L-DOPA (3.0 g, 15.2 mmoles, 1 equiv), TBDMSCl (6.8 g, 45.7 mmoles, 3 equiv), and anhydrous ACN (30 mL). The heterogenous mixture was cooled at 0° C. while being stirred vigorously and purged with N₂ gas continuously. To the cooled mixture, DBU (6.9 mL, 45.7 mmoles, 3 equiv) was added dropwise and N₂ purging was stopped. The resulting mixture was stirred at RT for 16 h and then filtered. The filtrate was cooled at 0-4° C. for 15 min, after which bis-O-TBDMS-protected DOPA crashed out of the liquid as an amorphous white solid. This product was collected by vacuum filtration, washed with cold EtOH (-20° C.). Subsequent crops of the product were collected from the mother liquor via the same process. The product batches were combined and dried to give pure bis-O-TBDMS-protected DOPA (5 g, 77% isolated yield), which was used in the next step.

(Step 2) A 100-mL round-bottom flask was charged with K₂CO₃ (4.2 g, 30.4 mmoles, 3 equiv), dioxane (20 mL) and water (10 mL). To the mixture cooled at 0° C., was added bis-O-TBDMS-protected DOPA (4.3 g, 10.0 mmoles, 1 equiv) and Boc₂O (2.4 g, 11.1 mmoles, 1.1 equiv). After being stirred at RT for 1 d, the reaction mixture was diluted with water (10 mL) and treated with acetic acid until the pH reached to ca. 4.7. The resulting mixture was extracted twice with CH₂Cl₂. The organic layers were washed with water and brine, dried over Na₂SO₄, and concentrated under reduced pressure to afford the title compound S1 (4.9 g, 75% isolated yield over 2 steps).

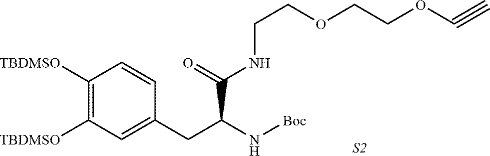 [SMILES: C#COCCOCCNC(=O)[C@H](Cc1ccc(O[Si](C)(C)C(C)(C)C)c(O[Si](C)(C)C(C)(C)C)c1)NC(=O)OC(C)(C)C ; "S2"]

Synthesis of tert-butyl (S)-(3-(3,4-bis((tert-butyldimethylsilyl)oxy)phenyl)-1-oxo-1-((2-(2-(prop-2-yn-1-yloxy)ethoxy)-ethyl)amino)propan-2-yl)carbamate (S2)

To a stirred mixture of S1 (3.4 g, 6.5 mmoles, 1.0 equiv) and EDC (1.5 g, 7.8 mmoles, 1.2 equiv) in CH₂Cl₂ (60 mL) at RT, was added a mixture of 2-[2-(2-propynyloxy)ethoxy]ethylamine (1.2 g, 8.4 mmoles, 1.3 equiv) and DMAP (0.2 g, 1.8 mmoles, 0.3 equiv) in CH₂Cl₂ (5 mL). After being stirred at RT for 12 h, the reaction mixture was quenched with water (20 mL). The resulting heterogenous mixture was extracted twice with CH₂Cl₂. The organic layers were washed with water and brine, dried over Na₂SO₄, and concentrated under reduced pressure. The resulting crude residue was purified by silica gel flash chromatography (CH₂Cl2/MeOH step gradient) to afford S2 (2.3 g, 55% isolated yield). R_{f}= 0.35 (CH₂Cl₂/MeOH 95:5). ¹H NMR (500 MHz, CDCl₃) δ 6.74 (d, J = 8.0 Hz, 1H), 6.66 (d, J = 2.0 Hz, 1H), 6.63 (dd, J = 8.0, 2.0 Hz, 1H), 6.26 (br t, J = 5.0 Hz, 1H), 4.95 (br s, 1H), 4.26 (br s, 1H), 4.19 (t, J = 2.5 Hz, 2H), 3.65 (ddd, J = 6.0, 4.0, 2.0 Hz, 2H), 3.59 (ddd, J = 6.0, 4.0 Hz, 2H), 3.52-3.35 (overlapping m, 4H), 2.94 (apparent d, J = 6.0, 2H), 2.44 (t, J = 2.5, 1H), 1.41 (s, 9H), 0.99 (s, 9H), 0.98 (s, 9H), 0.19 (overlapping s, 6H), and 0.18 (overlapping s, 6H). ¹³C-NMR (125 MHz, CDCl₃) δ 171.4, 155.5, 146.9, 146.0, 129.8, 122.3, 122.4, 121.2, 80.2, 79.7, 75.0, 70.3, 70.0, 69.2, 58.6, 55.9, 39.4, 38.0, 28.5, 26.1, 18.62, 18.60, -3.82, -3.85, -3.87, and -3.90. HRMS (ESI): Calculated for [C₃₃H₅₈N₂O₇Si₂ + Na+] 673.3675, found 673.3740. [a]²⁰_{D} = +7.5° (c = 4 g/100 mL, 0.04 g/mL, CHCl₃).

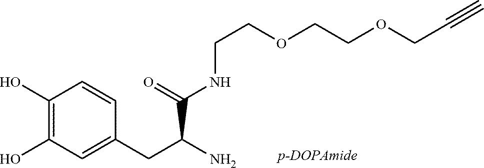

Synthesis of (S)-2-amino-3-(3,4-dihydroxyphenyl)-N-(2-(2-(prop-2-yn-1-yloxy)ethoxy)ethyl)-propanamide, Propargyl-2EG-DOPAmide (p-DOPAmide)

A 10-mL pressure flask was charged with S2 (1.5 g, 2.3 mmoles, 1 equiv), TFA (4 mL, 52.6 mmoles, 23 equiv), and Milli-Q water (0.2 mL, 11.1 mmoles, 5 equiv). The flask was sealed with a PTFE cap and placed in an oil bath heated at 45° C. The reaction solution was stirred for 4 h, then allowed to cool to RT, and was slowly added into cooled (-30° C.) and stirred Et₂O (60 mL). The resulting suspension was vortexed and the supernatant was removed. The remaining precipitate was washed twice with Et₂O and kept under high vacuum for 1 h. The resulting white solid essentially contained p-DOPAmide as an ammonium trifluoroacetate salt (0.8 g, 80% isolated yield) in a powder form with an opaque white color, which turns into a wax at RT upon handling. ¹H NMR (500 MHz, d₆-DMSO) δ 8.88 (s, 1H), 8.84 (s, 1H), 8.43 (br t, J = 5.0 Hz, 1H), 8.08 (br s, 3H), 6.66 (d, J = 8.0 Hz, 1H), 6.61 (d, J = 2.0 Hz, 1H), 6.47 (dd, J = 8.0, 2.0 Hz, 1H), 4.13 (d, J = 2.5 Hz, 2H), 3.83 (br t, J = 6.5 Hz, 1H), 3.55 (m, 2H), 3.51 (m, 2H), 3.42 (m, J = 2.5, 1H), 3.39 (m, 1H), 3.31 (m, 2H), 3.18 (m, 1H), 2.84 (dd, J = 14.0, 6.5, 1H), and 2.75 (dd, J = 14.0, 7.5, 1H). ¹³C NMR (125 MHz, d₆-DMSO) δ 168.1, 145.2, 144.2, 125.4, 120.1, 116.8, 115.5, 80.3, 77.2, 69.3, 68.8, 68.5, 57.5, 53.8, 38.7, and 36.6. HRMS (ESI): Calculated for trifluoroacetate-free product [C₁₆H₂₂N₂O₅ + H+] 323.1601, found 323.1594. LRMS (ESI): Calculated for ammonium trifluoroacetate salt product [C₁₈H₂₃F₃N₂O₇ + H+] 437.2, found 437.3. [a]²⁰_{D} = +16.0° (c = 3.0 g/100 mL, 0.030 g/mL, 1 M HCl).

Example 2: Synthesis of (5)-2-amino-3-(3,4-bis((tert-butyldimethylsilyl)oxy)phenyl)-N-(2-(2-(prop-2-yn-1-yloxy)ethoxy)ethyl)-propanamide (S3)

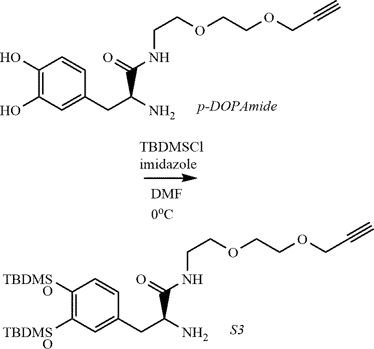

To a stirred solution of the trifluoroacetate salt of p-DOPAmide (116 mg, 0.27 mmoles, 1.0 equiv) in anhydrous DMF (0.7 mL) at 0° C., was added TBDMSCl (140 mg, 0.93 mmoles, 3.5 equiv) and imidazole (275 mg, 4.1 mmoles, 15.0 equiv). After being stirred for 12 h, the reaction mixture was diluted with CH₂Cl₂ (10 mL) and stirred with 1 M NaHCO₃ solution (2 mL) and 1 M NaCl solution (3 mL). The resulting emulsion was vortexed and the aqueous layer was discarded. The isolated organic layer was washed with 1 M NaCl solution (3 mL) twice and concentrated under reduced pressure. The resulting crude residue was purified by silica gel flash chromatography (CH₂Cl₂/MeOH step gradient) to afford S3 (138 mg, 93% isolated yield) in a colorless oil form. R_{f}═ 0.25 (CH₂Cl₂/MeOH 97:3). ¹H NMR (500 MHz, CDCl₃) δ 7.56 (br t, J = 5.5 Hz, 1H), 6.76 (d, J = 8.0 Hz, 1H), 6.70 (d, J = 2.0 Hz, 1H), 6.65 (dd, J = 8.0, 2.0 Hz, 1H), 4.21 (d, J = 2.5 Hz, 2H), 3.69 (ddd, J = 10.0, 4.0, 1.0 Hz, 2H), 3.65 (ddd, J = 10.0, 4.0, 1.0 Hz, 2H), 3.57 (t, J = 5.0 Hz, 2H), 3.52 (dd, J = 4.0, 1.0 Hz, 1H), 3.47 (m, 2H), 3.16 (dd, J = 14.0, 4.0, 1H), 2.51 (dd, J = 14.0, 4.0, 1H), 2.43 (t, J = 2.5, 1H), 1.36 (br s, 2H), 0.984 (s, 9H), 0.982 (s, 9H), 0.191 (overlapping s, 6H), and 0.189 (overlapping s, 6H). ¹³C NMR (125 MHz, CDCl₃) δ 174.7, 147.1, 145.9, 131.2, 122.5, 122.2, 121.2, 79.8, 74.8, 70.3, 70.1, 69.2, 58.7, 56.9, 40.6, 39.0, 26.14, 26.13, 18.6, -3.85 (overlapping 2 CH₃), -3.87 (CH₃), and -3.88. HRMS (ESI): Calculated for [C₂₈H₅₀N₂O₅Si₂ + Na+] 573.3150, found 573.3161. [a]²⁰_{D} = -21.1 ° (c = 1.8 g/100 mL, 0.018 g/mL, CHCl₃).

Example 3: Synthesis of 7-hydroxy-3-(4-((2-methoxyethoxy)methyl)-1H-1,2,3-triazol-1-yl)-2H-chromen-2-one (S4)

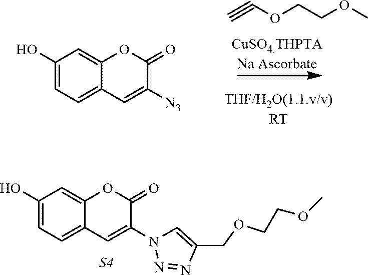

To a stirred solution of 3-Azido-7-hydroxycoumarin (77 mg, 0.38 mmoles, 1.0 equiv) in THF (6 mL) and water (6 mL) in a 20-mL amber vial, was added 3-(2-methoxyethoxy)prop-1-yne (86 mg, 0.76 mmoles, 2.0 equiv), CuSO₄ (30 mg, 0.19 mmoles, 0.5 equiv), and THPTA (83 mg, 0.19 mmoles, 0.5 equiv). The resulting homogenous liquid was purged with N₂ gas for 5 min. Sodium ascorbate (75 mg, 0.38 mmoles, 1.0 equiv) was added and the liquid was purged with N₂ gas for another 5 min, immediately after which the vial was sealed. The reaction mixture (~12 mL) was stirred at RT for 12 h and concentrated down to 1 mL under reduced pressure and by successive additions of acetonitrile to help remove bulk water. The resulting liquid was diluted with CH₂Cl₂ (2 mL) and purified by silica gel flash chromatography (CH₂Cl₂/MeOH step gradient) to afford S4 (107 mg, 88% isolated yield) as a liquid with a lime green color that glows. R_{f} = 0.35 (CH₂Cl₂/MeOH 97:3). ¹H NMR (500 MHz, d₆-DMSO) δ 8.59 (s, 1 H), 8.52 (s, 1H), 7.74 (d, J = 8.5, 1H), 6.91 (dd, J = 8.5, 2.0, 1H), 6.85 (d, J = 2.0, 1H), 4.62 (s, 1 H), 3.61 (m, 1 H), 3.48 (m, 1 H), and 3.25 (m, 3 H). ¹³C NMR (125 MHz, d₆-DMSO) δ 162.5, 156.3, 154.6, 144.1, 136.4, 124.9, 119.3, 114.3, 110.3, 102.2, 71.1, 68.9, 63.2, and 58.1. HRMS (ESI): Calculated for [C₁₅H₁₅N₃O₅ -(H+)] 316.0939, found 316.0938.

### Example 4: Synthetic Design, Surface Functionalization, and Characterization

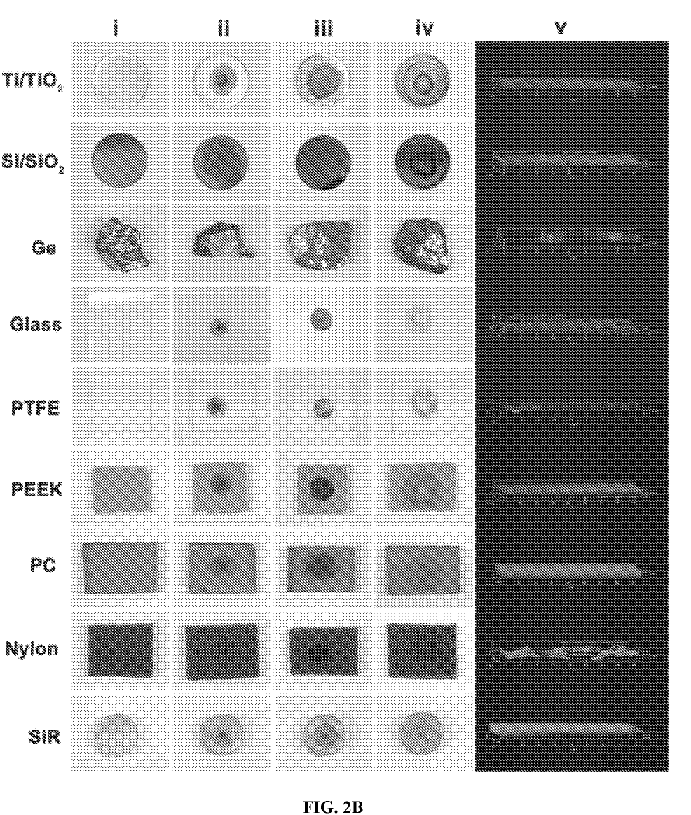

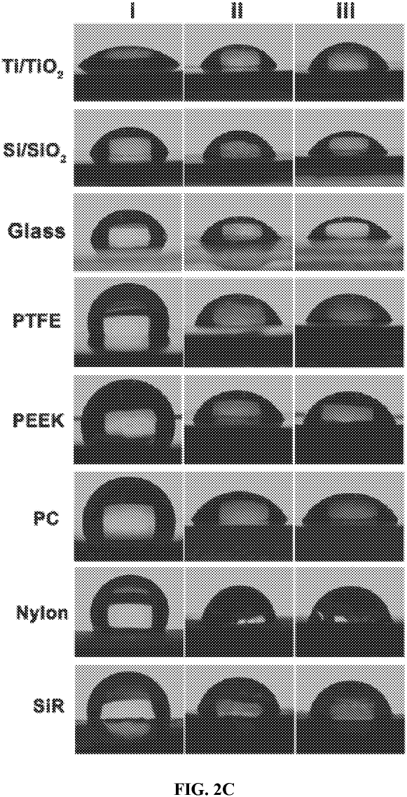

The synthesis of p-DOPAmide (FIG. 1A), which is an _{L}-DOPA-derived monomer that can polymerize while undergoing click reaction with a MOI-N₃ in the presence of copper ions (FIG. 1B), has been described herein. Generally, p-DOPAmide, a MOI-N₃ (e.g. TAMRA-N₃ or 3-N₃-7-hydroxycoumarin), a Cu(II) salt (e.g. CuSO₄), and a copper ligand (e.g. THPTA), are combined to provide a MCM, which is dropped onto a material substrate to attain a specific chemical or biological functionality. To explore the material independence of the surface functionalization, dye containing MOI-N₃ species (e.g. TAMRA-N₃ or 3-N₃-7-hydroxycoumarin) were grafted onto a wide variety of substrates, including metal and metal oxide (Ti/TiO₂), a ceramic (glass), semiconductors (Si/SiO₂ and Ge), and polymers (PTFE, PEEK, PC, PU, nylon, and SiR) (FIGS. 2A-2C). In certain embodiments, the resulting coating was visible as a brown-colored thin film (referred to as coated), which altered the surface wettability of the original material (referred to as bare) (FIG. 2A). The functionalized substrates remained stable unless scratched, treated by aggressive ultrasound, or subjected to strong acid (pH < 1) or strong base (pH > 10).

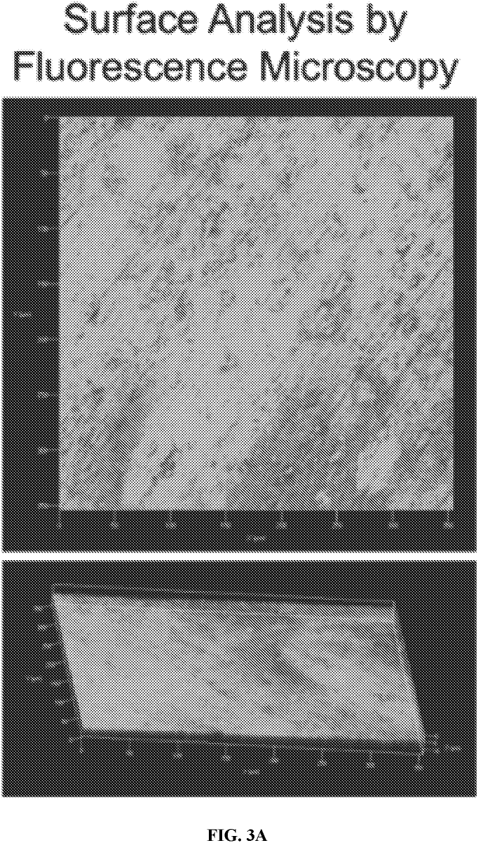

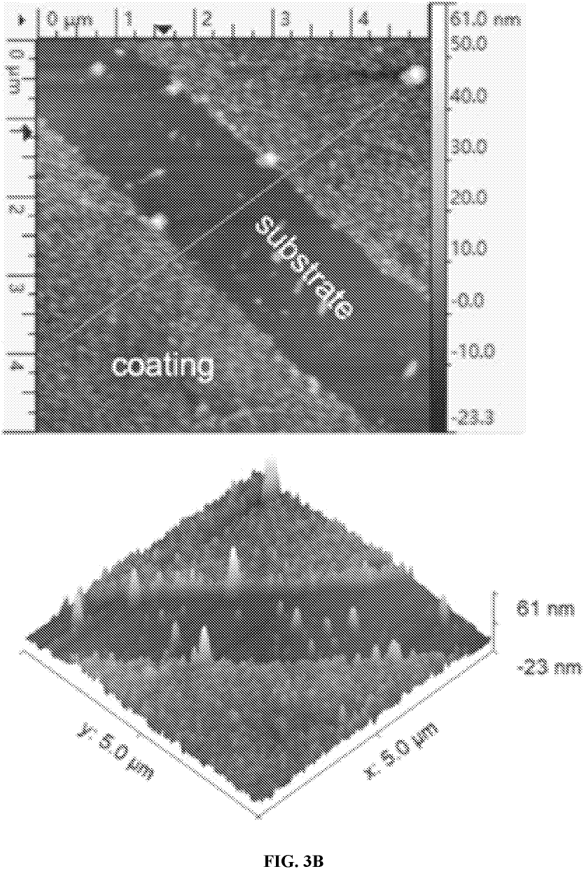

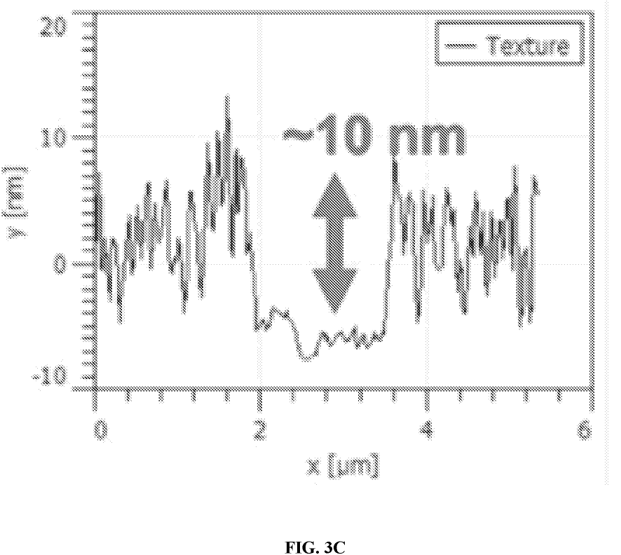

In certain embodiments, the 5′-N₃-DNA-3′-FAM was used as the MOI-N₃, providing coating comprising FAM tagged DNA. The DNA coating exhibited a uniform green fluorescence signal from the FAM group with a 488 nm excitation, and further exhibited a nanorough surface topography, with a thickness of approximately 10 nm, as determined by AFM. (FIGS. 3A-3C).

The coatings formed using a mixture of only CuSO₄, THPTA, and p-DOPAmide had a brown color under ambient light, which is a characteristic physical change which occurs during catechol polymerization (FIGS. 2A-2B). In certain embodiments TAMRA-N₃ was used as the MOI-N₃, giving rise to red/maroon coatings on the substrates (FIG. 2B). In certain embodiments 3-N₃-7-hydroxycoumarin was used as the MOI-N₃. In such embodiments, the incorporation of the coumarin was confirmed by fluorescence detection at 405 nm excitation. (FIGS. 2A-2B). Free 3-N₃ coumarins typically exhibit a low fluorescence intensity with 405 nm excitation wavelength, but once they undergo a click reaction with alkynes, the resulting 3-triazole-coumarin products display a substantial increase in fluorescence quantum yield.

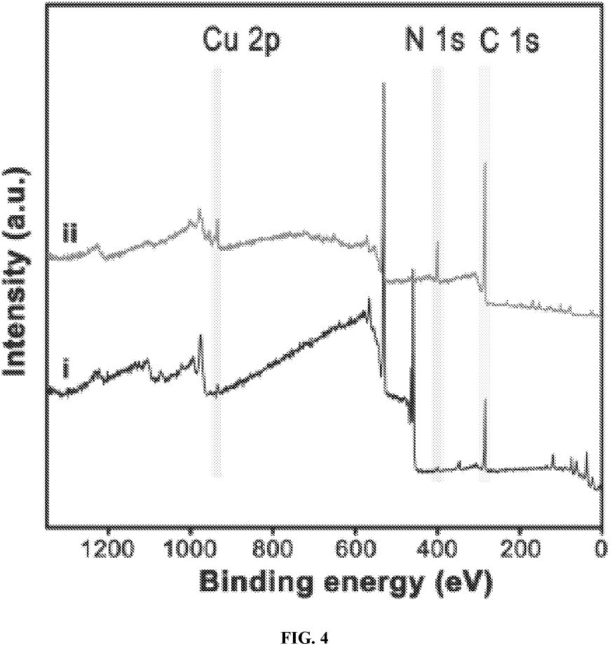

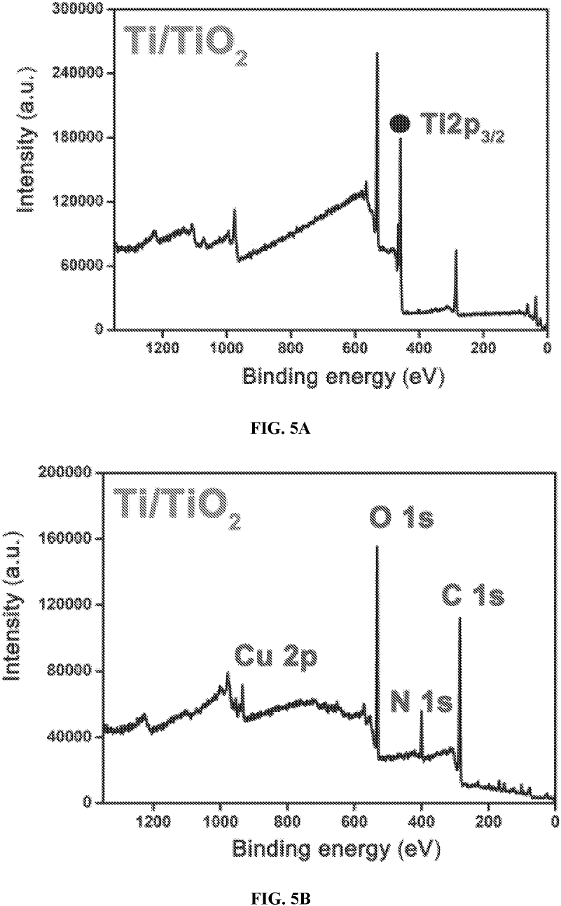

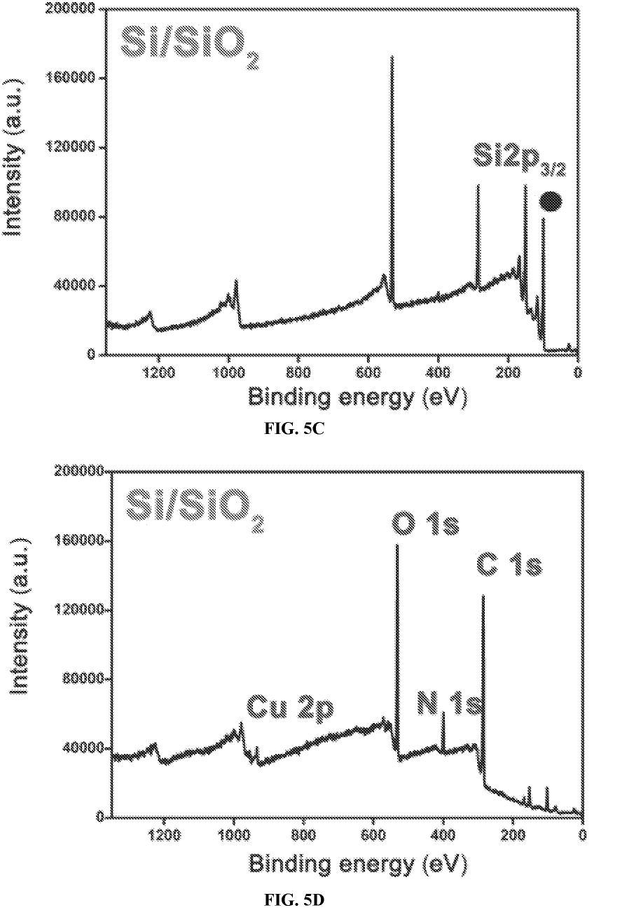

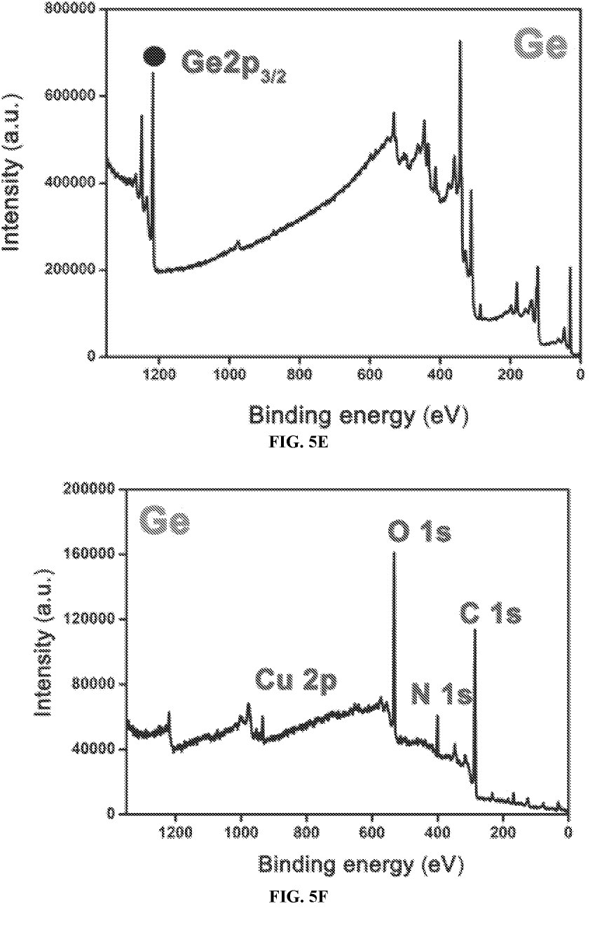

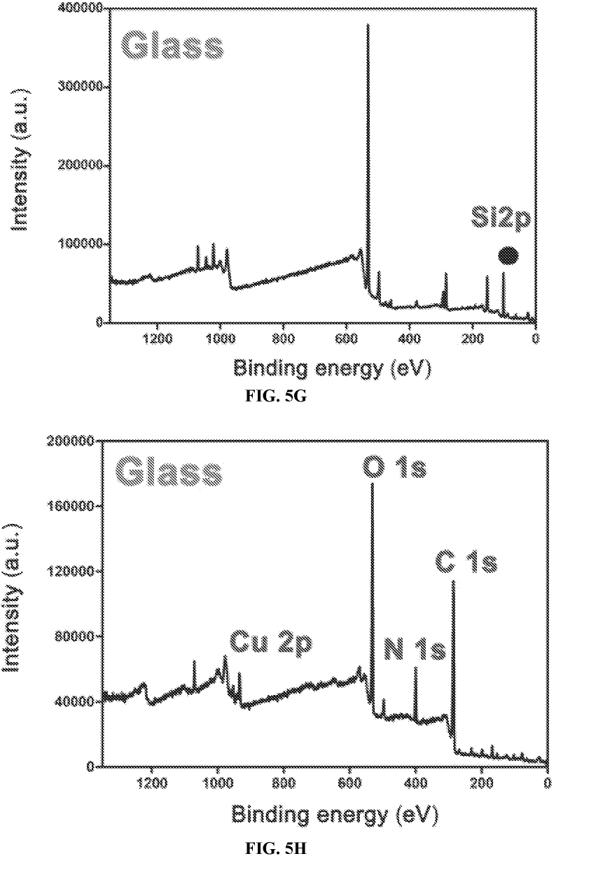

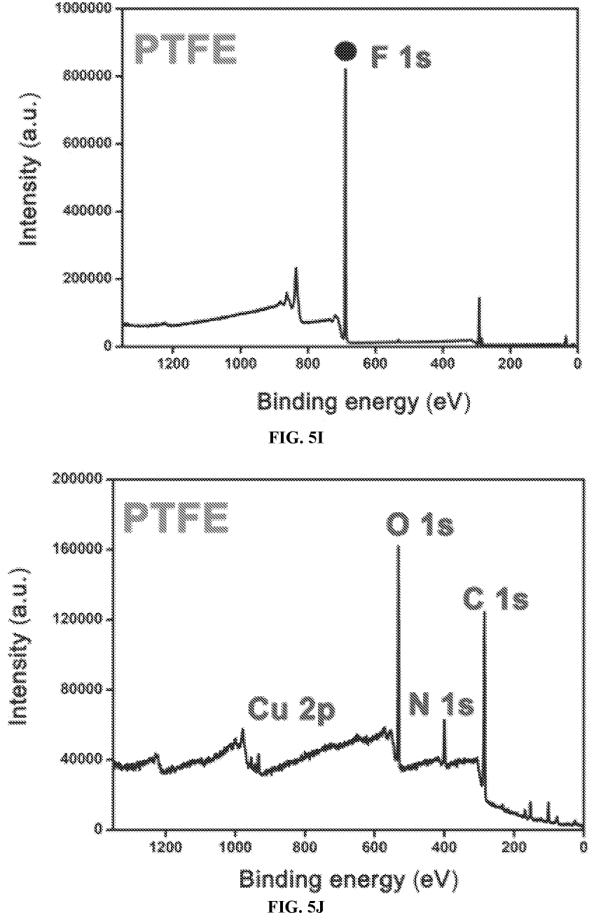

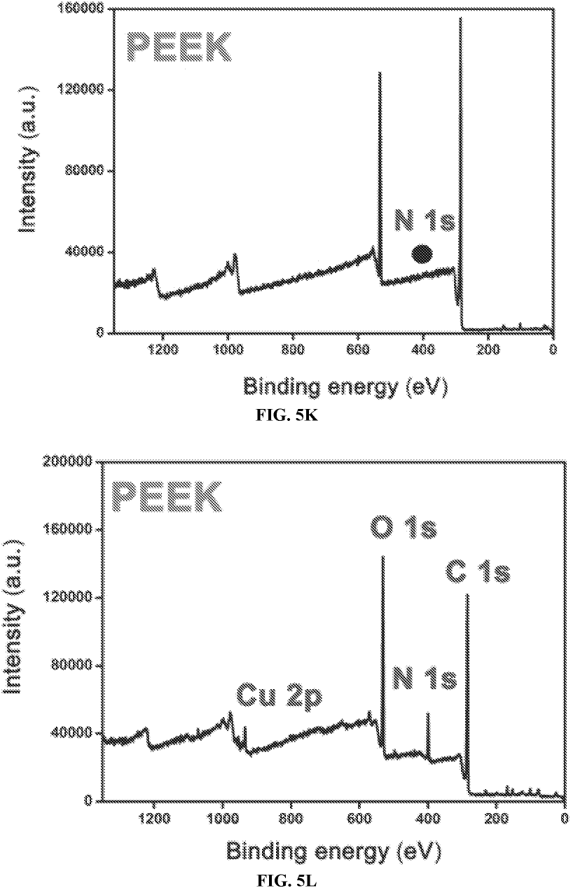

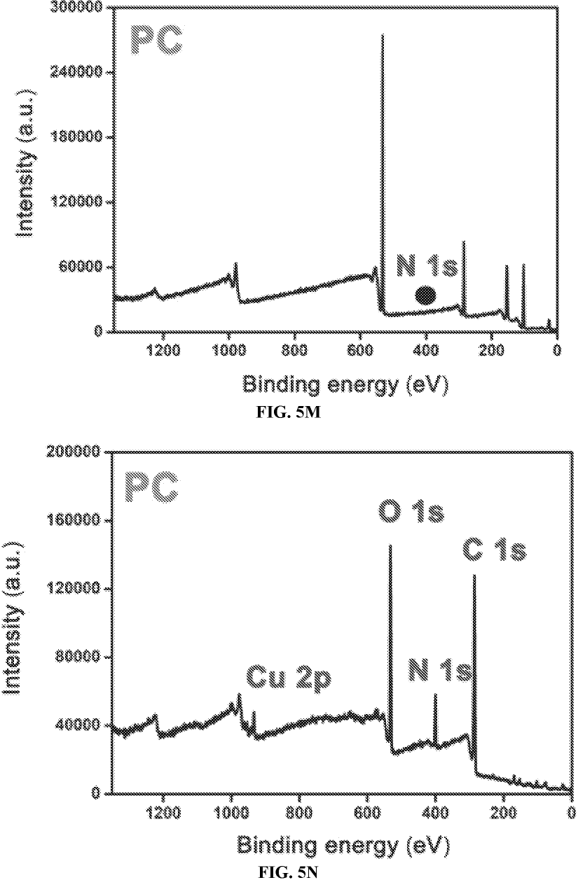

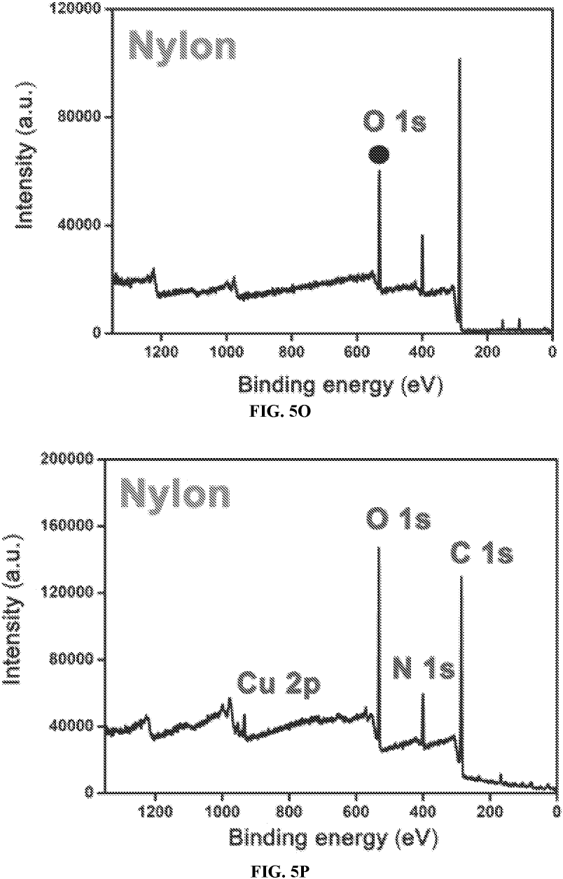

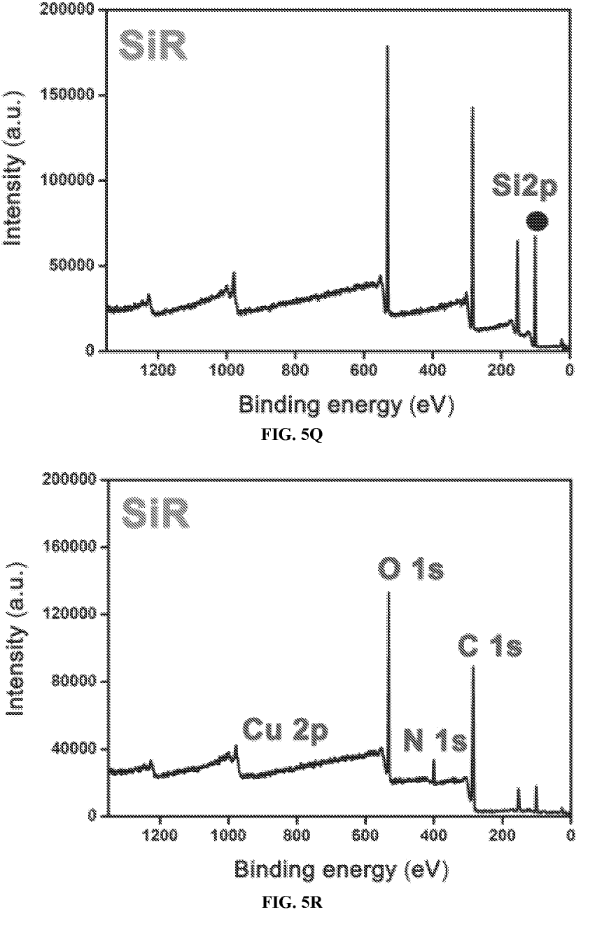

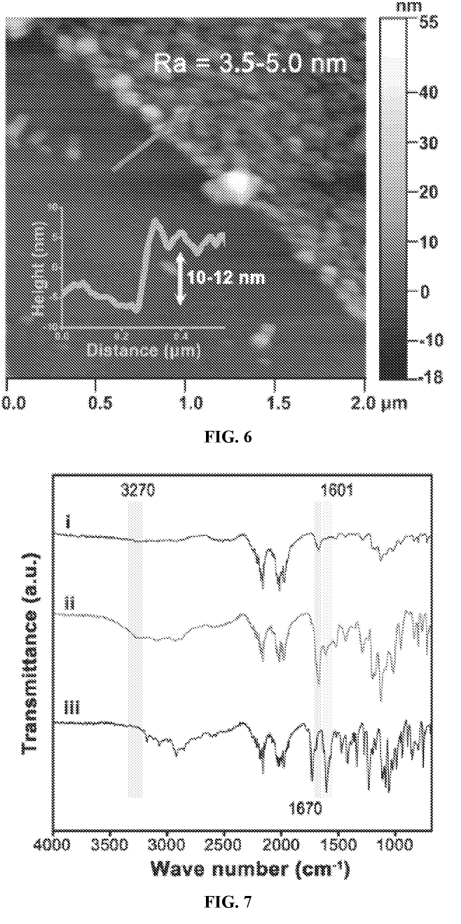

In addition, drop coating altered the surface wettability of the substrate materials (FIG. 2C). The coatings were confirmed by XPS (FIG. 4 and FIGS. 5A-5R). AFM investigations showed coatings with 10-20 nm thickness and 3.5-5.0 nm roughness (FIG. 6). The existence of MOI-clicked coatings was validated by ATR-FTIR (FIG. 7). The ATR-FTIR spectra of Ti/TiO₂ surfaces coated with p-DOPAmide, Cu(II), and THPTA, with or without 3-N₃-7-hydroxycoumarin shared similarities in the band range of 700-1580 cm⁻¹, which are correlated with N-H, C-N, and C-O vibrations (FIG. 7). For the coating with the MCM (i.e. comprising the MOI), a new absorption peak emerged at 1601 cm⁻¹, corresponding to the triazole N=N stretching, the heterocycle that forms from azide-alkyne cycloaddition. This also agrees with the absorption maximum at 1601 cm⁻¹ observed for the reference compound S4, which mimics the desired click reaction product. Additionally, the band at 1670 cm⁻¹, present in coatings with and without the MOI, shows O=C-N stretching, as expected from the amide group of a p-DOPAmide assembly. Finally, the band at 3270 cm⁻¹ agrees with the weak N-H/O-H stretching reported for catechols.

Taken together, these results demonstrate that the single-step drop coating described herein led to material-independent functionalization of solid surfaces.

### Example 5: Kinetics and Mechanism Studies

### Kinetics of Coating

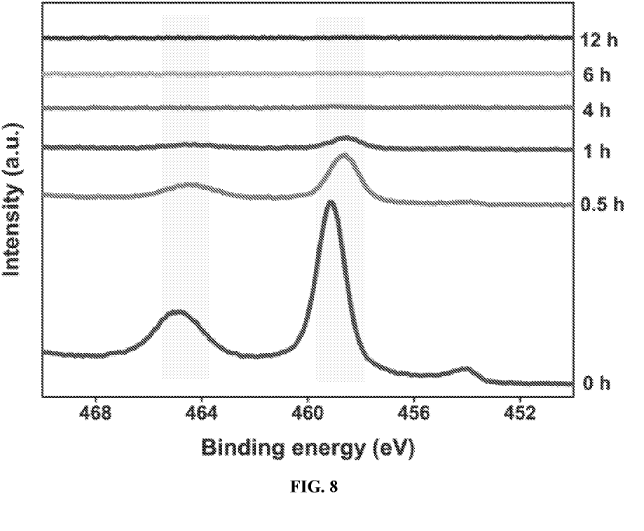

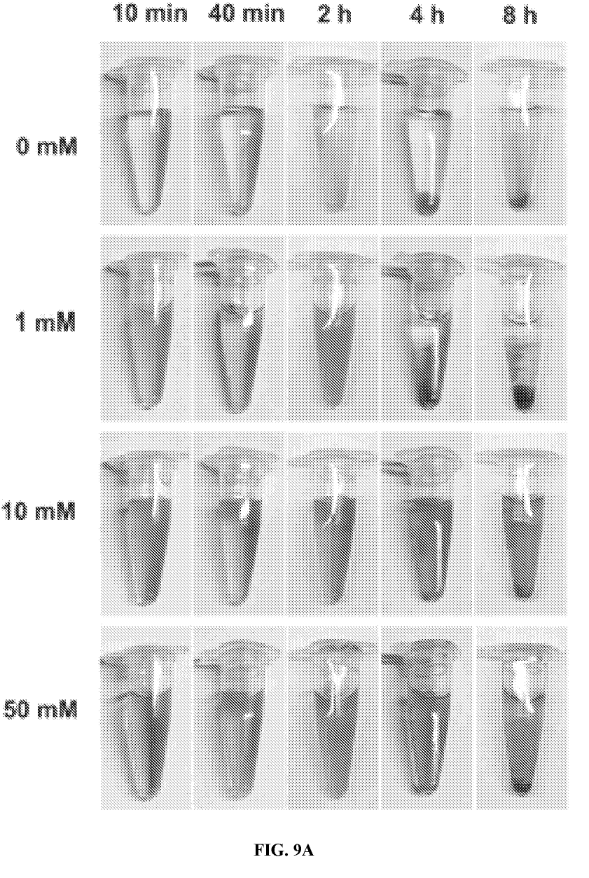

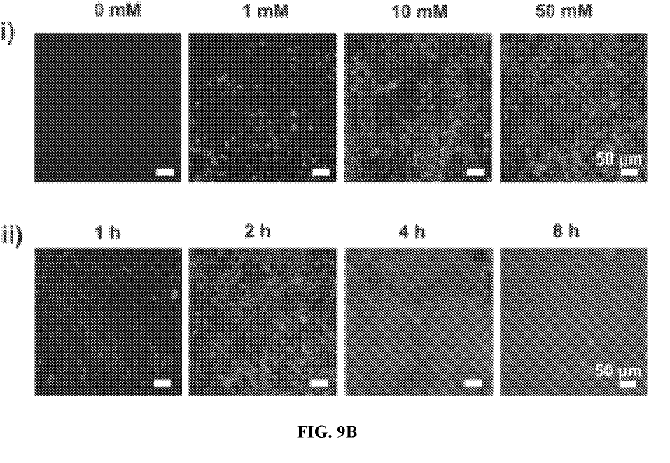

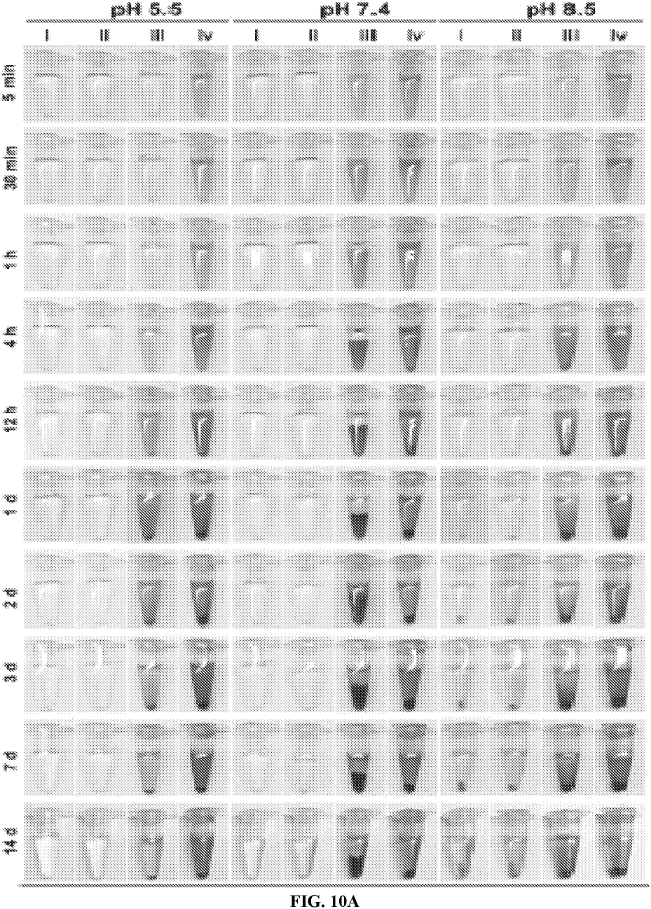

To understand the kinetics of the surface functionalizations, the progress of molecule attachment on a Ti/TiO₂ surface was investigated. A nanolayer coating rapidly formed across a 10 mm substrate by incubating its surface with 100 µL of the MCM (i.e. MOI-N₃ = 3-N₃-7-hydroxycoumarin). Substrate XPS signals of Ti 2p decreased substantially within the first 30 min and became almost invisible after 1 h, which is indicative of a rapid increase in coating thickness (FIG. 8). Greater p-DOPAmide concentration both accelerated p-DOPAmide polymerization (FIG. 9A) and increased 3-N₃-7-hydroxycoumarin grafting density (FIG. 9B).

### Mechanism of Coating

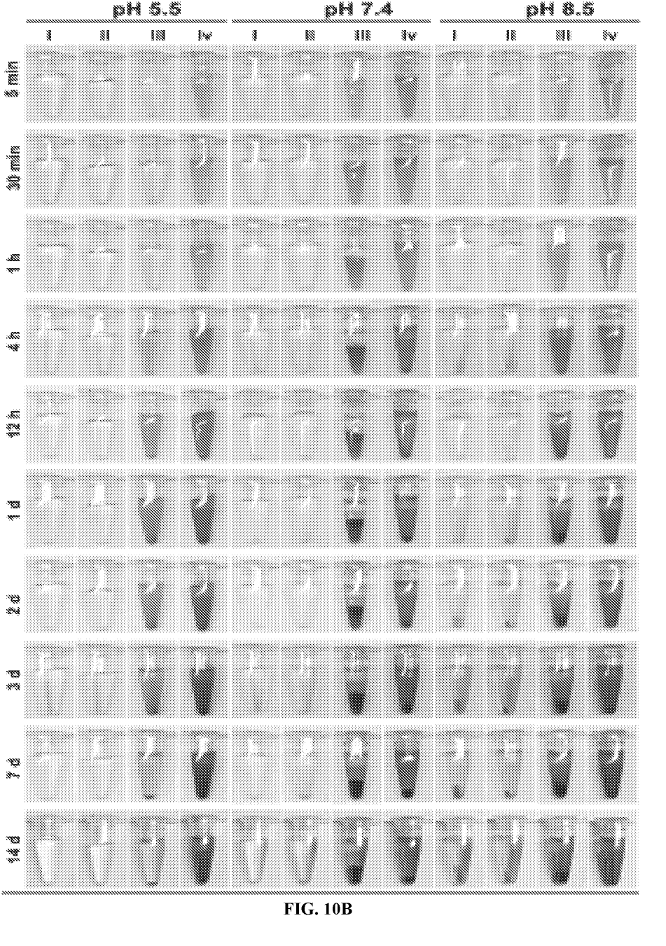

The effects of Cu(II), THPTA, and their combination on the polymerization of p-DOPAmide at different pHs (i.e. pH = 5.5, 7.4, and 8.5) were evaluated (FIGS. 10A-10B). In the absence of Cu(II) and THPTA, oxidation of p-DOPAmide was more significant at higher pH. This pH-dependence is similar to that reported for oxidative polymerization of _{L}-DOPA or DA. Furthermore, mixtures without CuSO₄ developed a lighter color at pH 8.5 (FIGS. 10A-10B). CuSO₄ and CuSO₄-THPTA drastically increased the rate of p-DOPAmide oxidation.

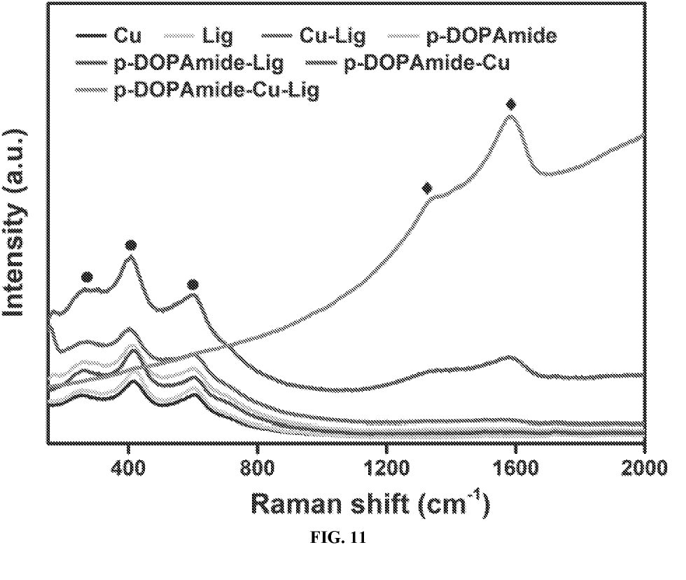

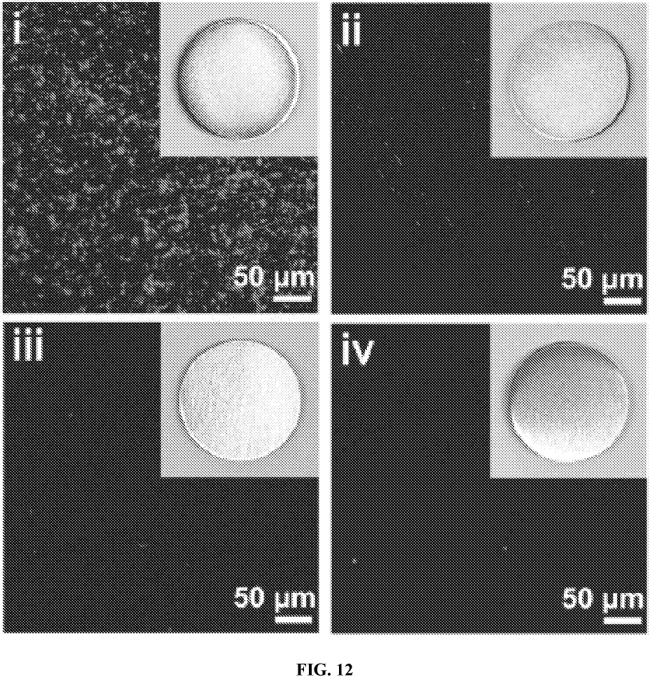

The effects of Cu(II) and THPTA on coating formation were also examined by Raman spectroscopy (FIG. 11). The introduction of Cu(II) into p-DOPAmide solutions led to polymers with vibrational peaks at 1335 and 1580 cm⁻¹, which are similar to the signals characteristic for aromatic stretching of catechols and carbonyl stretching of quinones, respectively. These peaks were more pronounced in the presence of THPTA.

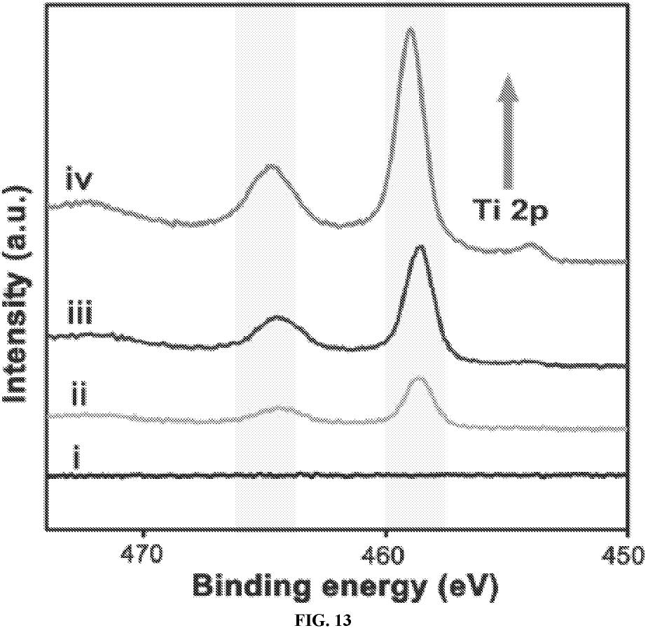

The impact of other additives and protecting groups on the coatings were also investigated. Supplementation of the MCM with FeCl₃ (0.05 or 0.5 equiv), which forms Fe(III)-catechol complexes, but does not assist catechol oxidation, decreased surface colorization and fluorescence, and led to the emergence of XPS signals of Ti 2p (FIG. 12). Additionally, the coating and grafting were greatly inhibited when p-DOPAmide was replaced with compound S3, wherein the cateochol hydroxyl groups are protected as the tertbutyldimethylsilyl (TBDMS) ethers (FIGS. 12-13). These results provide further support that the mechanism of coating depends upon the oxidation of p-DOPAmide, which is in agreement with literature reports of catechol binding of Cu(II) via chelation with subsequent oxidation to provide semiquinones.

Upon combining the MCM components, the MCM color changes promptly from light green (likely due to Cu(II) salts) to light brown (indicative of a polymer from catecholamine), which progressively darkens. This suggests that there is residual molecular oxygen sufficient to induce catechol oxidation in the presence of Cu(II), which leads to polymerization prior to material surface contact.

### Mechanism of Grafting

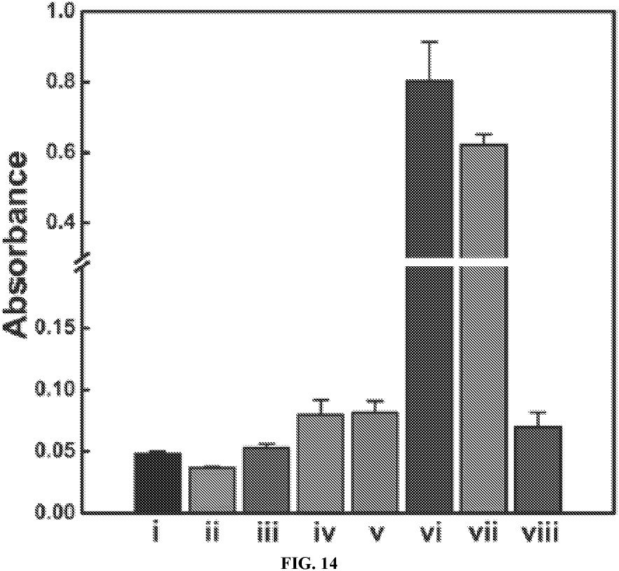

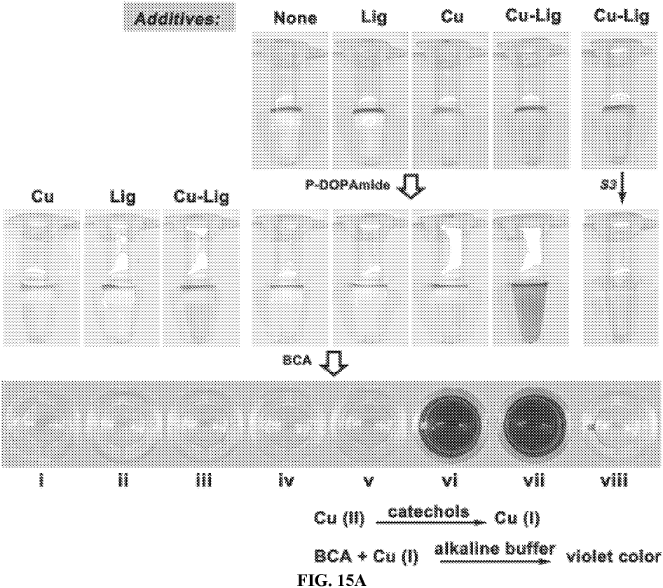

The copper species that catalyzes the click reaction is widely regarded to be Cu(I). Thus, it was reasoned that the Cu(II) species employed in the method described herein (e.g. CuSO4) may be converted to Cu(I) in situ during the surface functionalization. Thus, a modified micro bicinchonic acid (microBCA) assay was performed (FIG. 14 and FIGS. 15A-15B). The microBCA assay relies on the reduction of Cu(II) to Cu(I) by proteins or other reductants in an alkaline buffer, wherein Cu(I) binds BCA and forms a complex that has a violet color. Upon addition of BCA to a solution comprising p-DOPAmide and CuSO₄ a violet color was formed. Conversely, under conditions in which S3 was used instead of p-DOPAmide no color formation was observed (FIGS. 15A-15B). Thus, the aryl hydroxyl groups present in the catechol species are likely involved in the reduction of Cu(II) to Cu(I). When p-DOPAmide was introduced into a solution comprising CuSO₄ and THPTA, the color of the resulting mixture rapidly darkened substantially (FIGS. 15A-15B).

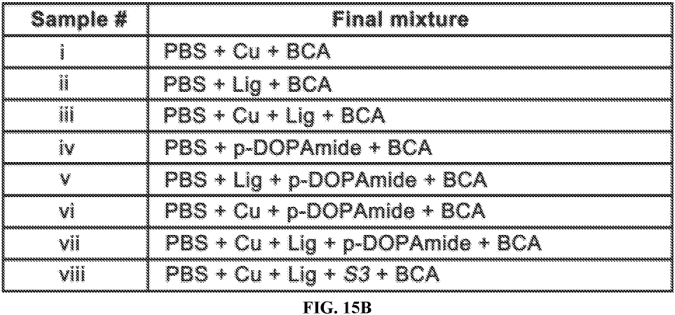

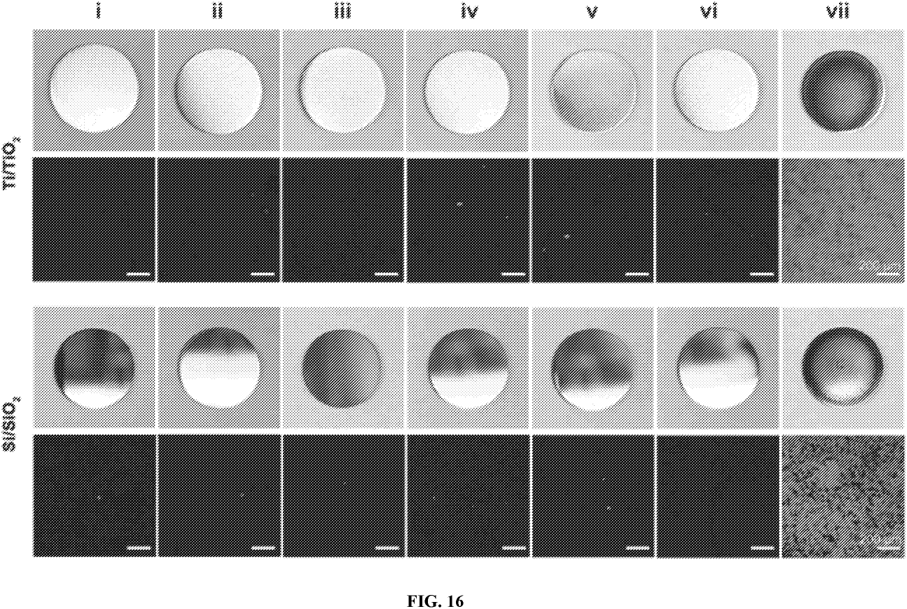

Additionally, Ti/TiO₂ and Si/SiO₂ substrates were drop coated using mixtures composed of TAMRA-N₃ and at least one of the following compounds: p-DOPAmide, CuSO₄, and THPTA (FIG. 16). For both substrate types, TAMRA fluorescence was detected only when all these compounds were present in the mixture, suggesting that at least one of the coating formation and click reaction are dependent on interactions amongst Cu(II), THPTA, and p-DOPAmide.

Taken together, these results suggest that Cu(II) oxidizes p-DOPAmide and is reduced to Cu(I), which can complex with THPTA and catalyze the click reaction between MOI-N₃ and the propargyl group of p-DOPAmide either in its monomeric or polymerized form. Therefore, it is conceivable that redox chemistry between p-DOPAmide and Cu(II) enables the simultaneous progression of both surface coating and click-mediated grafting.

### Film Formation

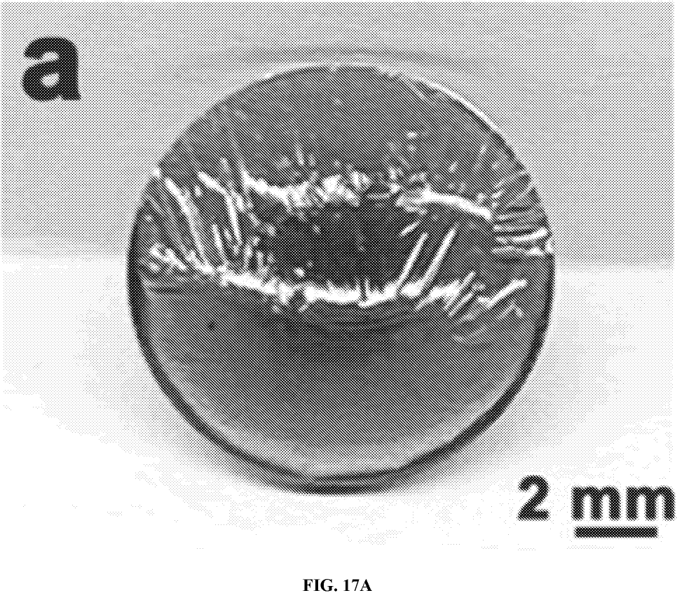

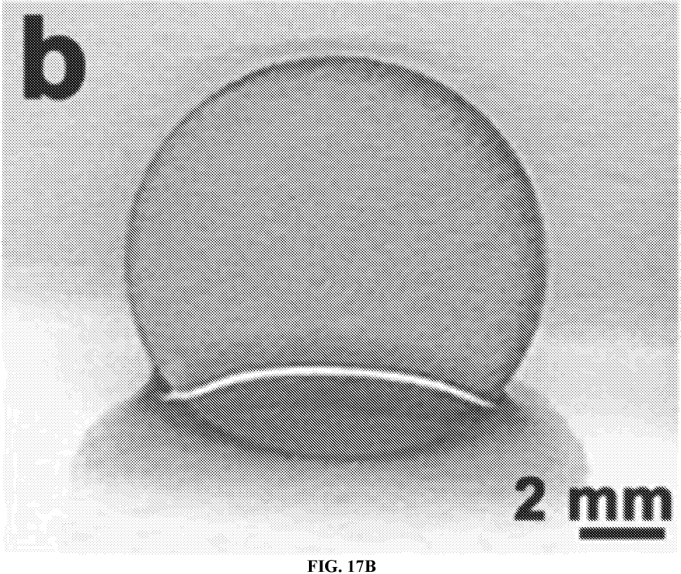

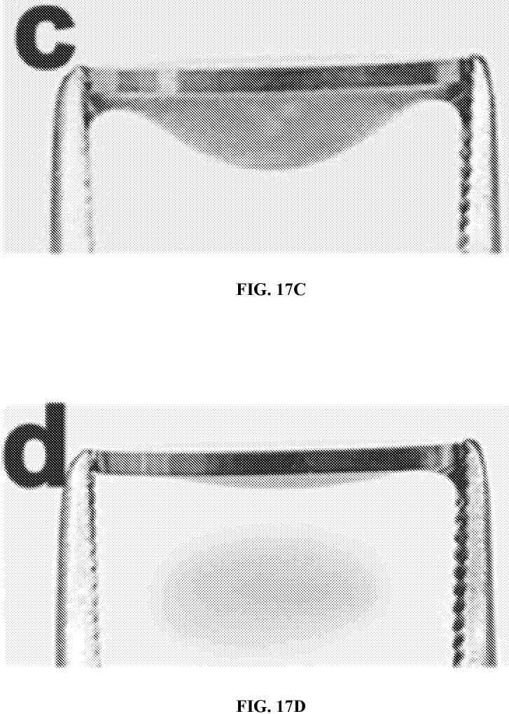

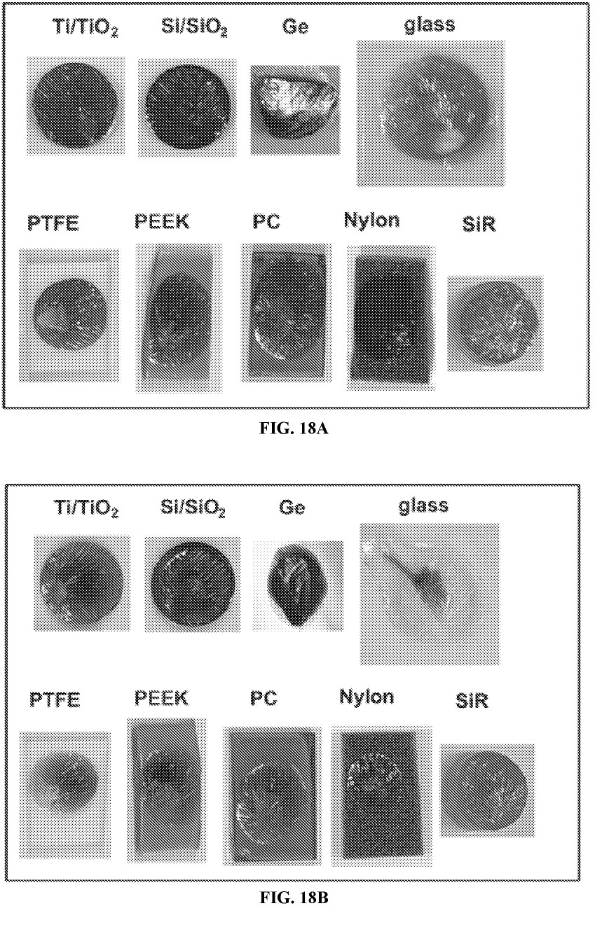

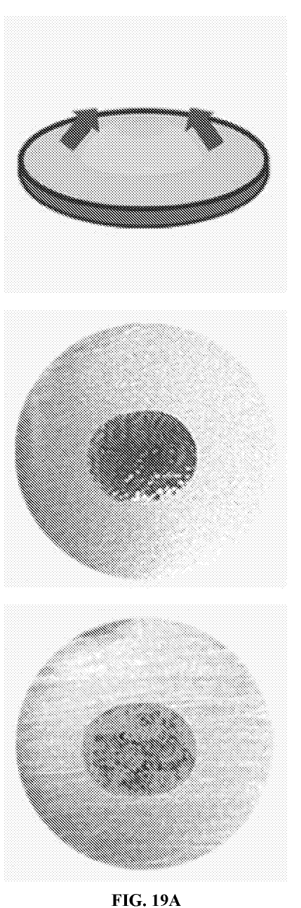

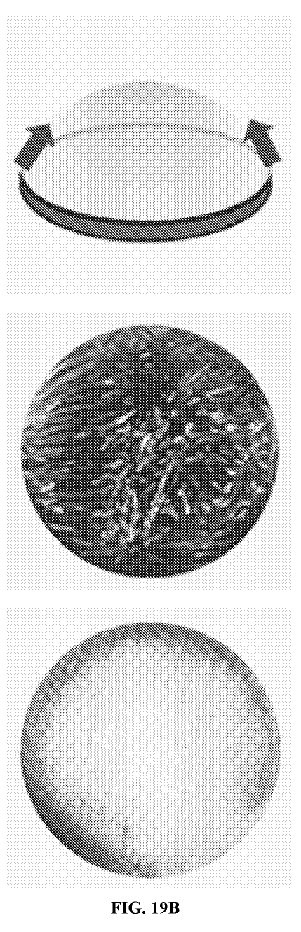

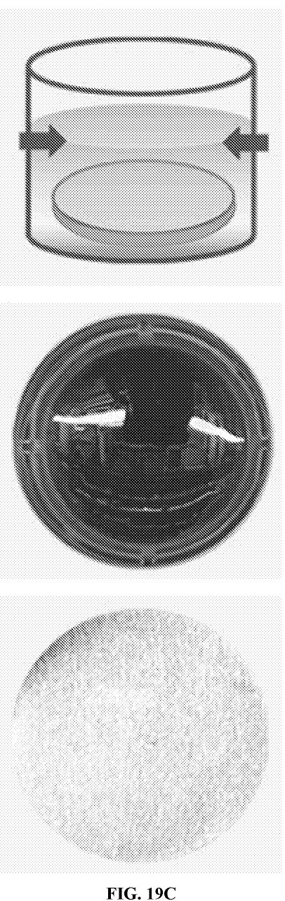

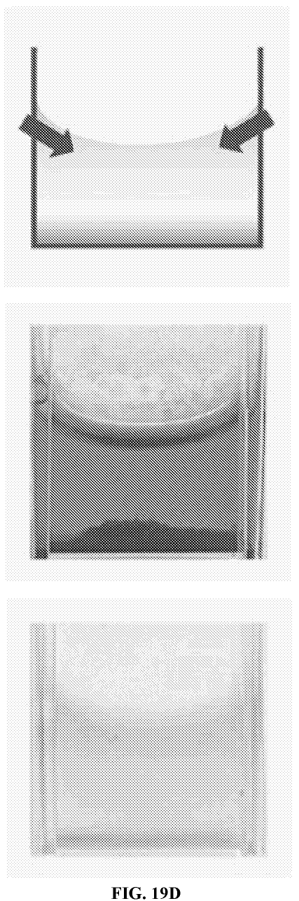

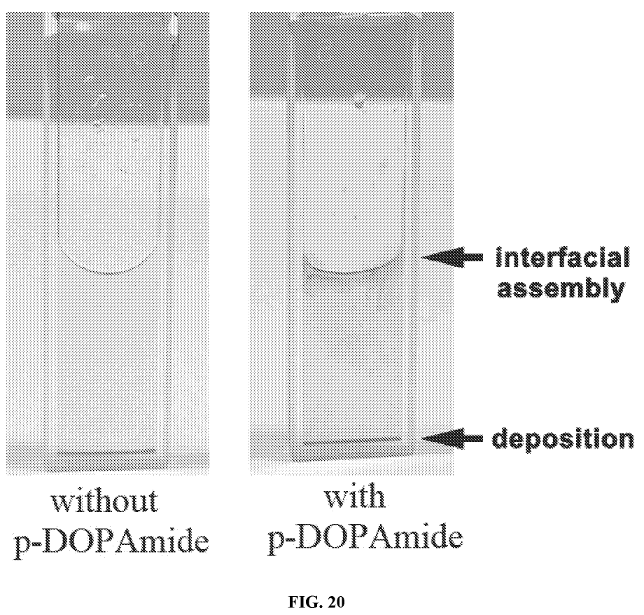

Incubation with the MCM leads to the formation of a film on material substrates during surface functionalization, likely because p-DOPAmide, or derivatives thereof, coordinate with Cu(II) and form a polymer-metal network of films (FIGS. 17A-17D and FIGS. 18A-18B). Formation of such a film occurs at the liquid/air interface (FIGS. 19A-19D and FIG. 20).

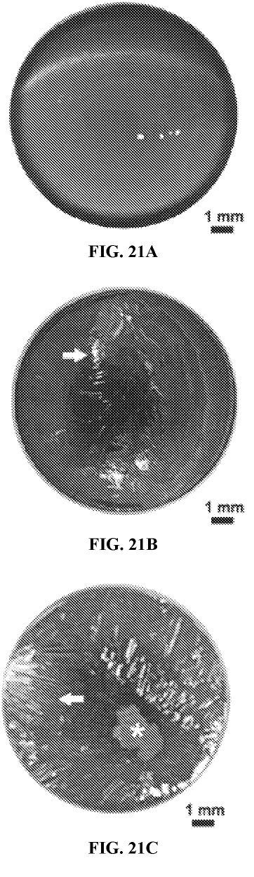

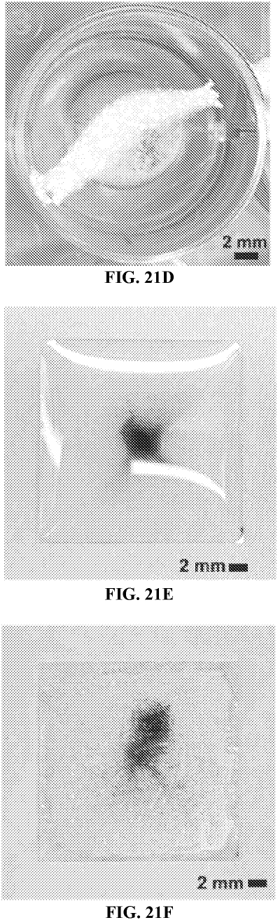

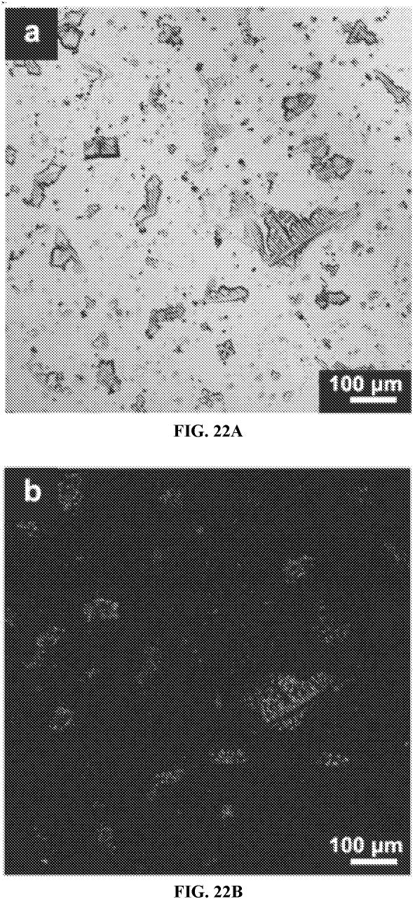

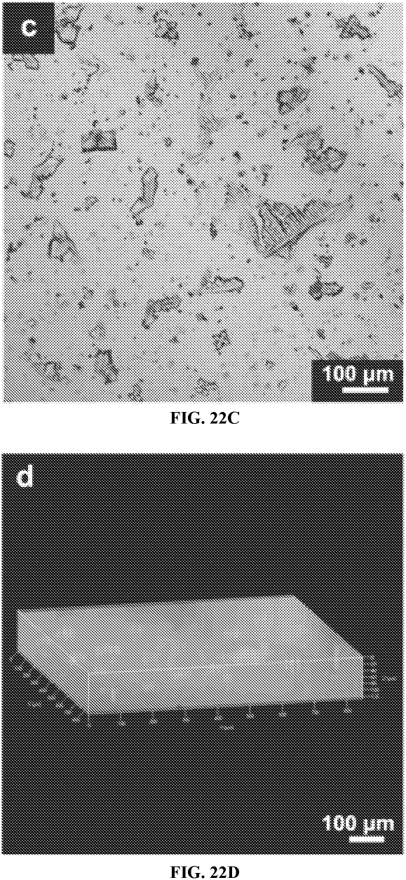

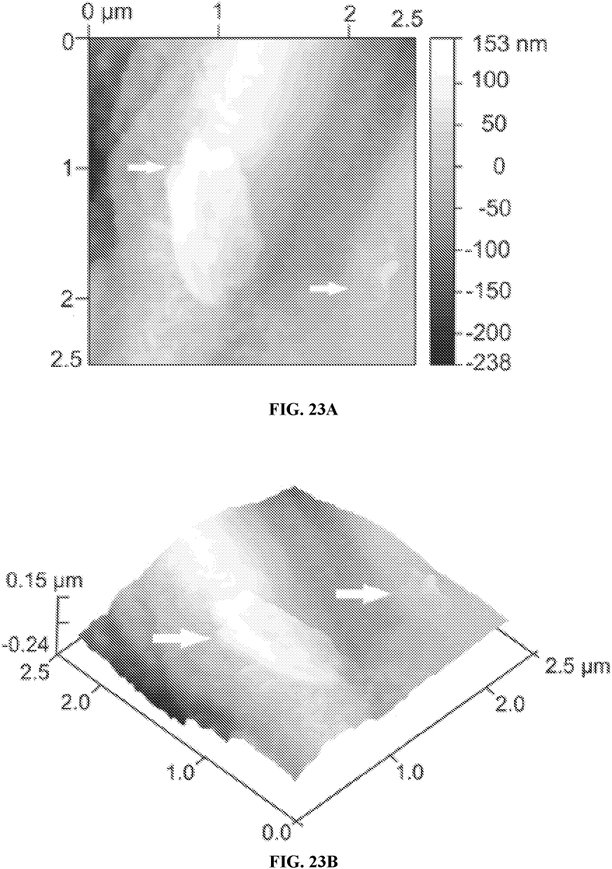

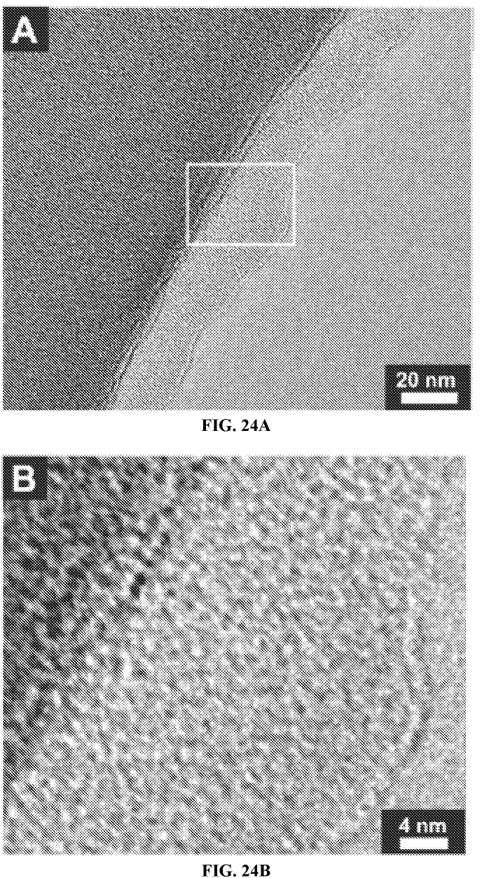

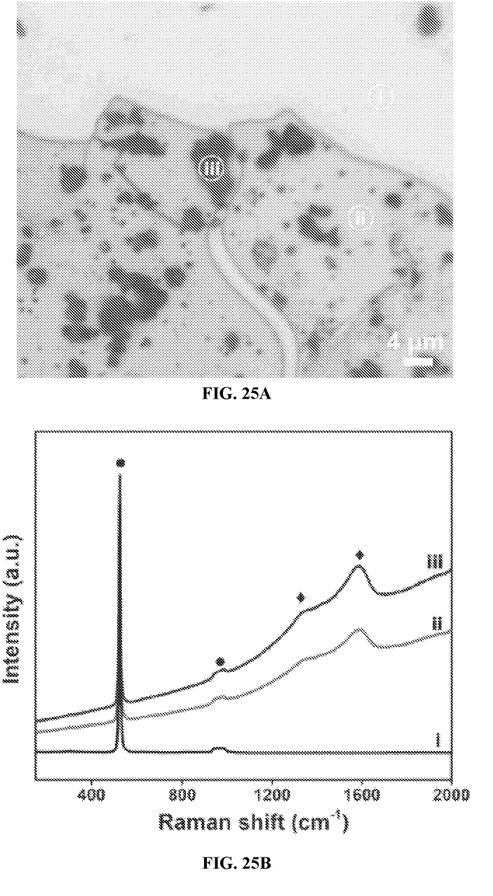

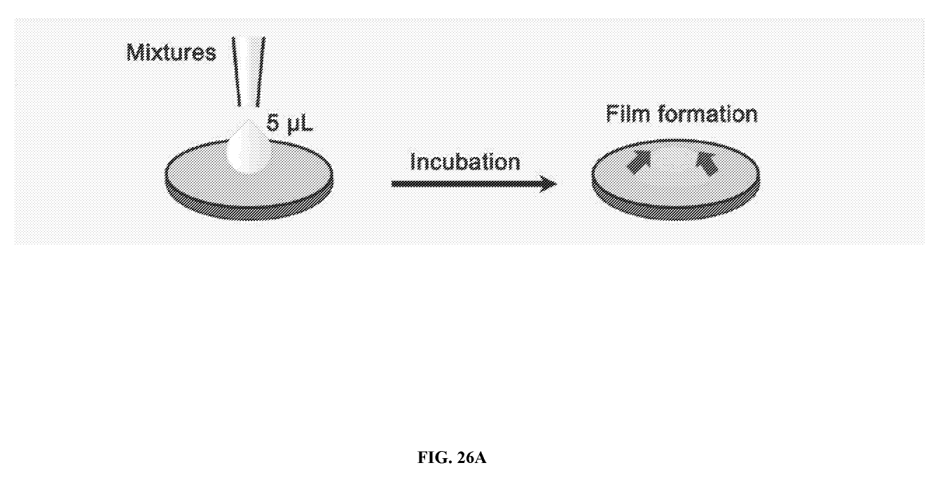

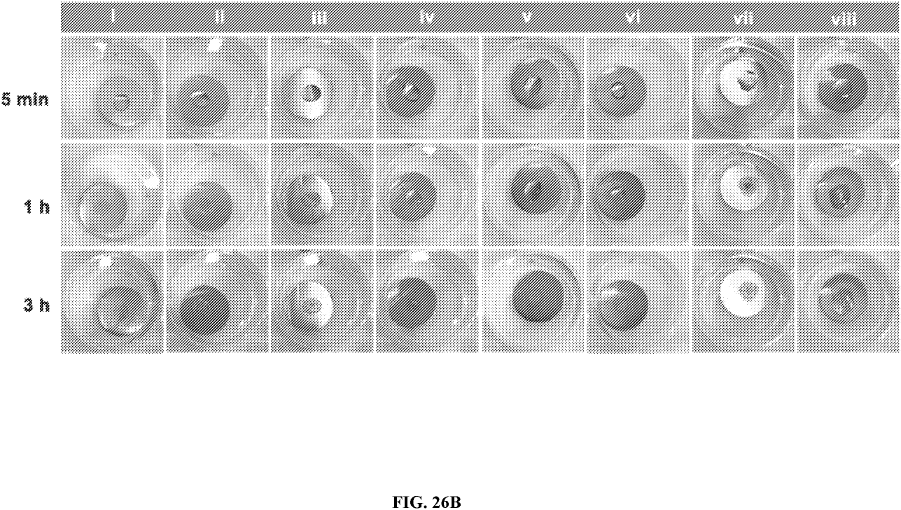

Film debris, collected by washing a Ti/TiO₂ substrate that was grafted with coumarin, exhibited fluorescence at 405 nm excitation (FIGS. 21A-21F and FIGS. 22A-22D). Nanometer-thick film debris was observed by AFM to have adhered to the substrate surface (FIGS. 23A-23B). Film formation on Si/SiO₂ was also examined. Such film debris was found to be amorphous based on TEM (FIGS. 24A-24B), and it provided peaks characteristic of catechols (1335 and 1580 cm⁻¹) by Raman spectroscopy (FIG. 25B). For the drop coated substrates, the rate of solvent evaporation was slower when Cu(II), THPTA, and p-DOPAmide were used, with or without MOI-N₃ (FIGS. 26A-26B), likely due to the formation of a film at the droplet liquid/air interface.

### Example 6: Distinctive Features of Drop Coating

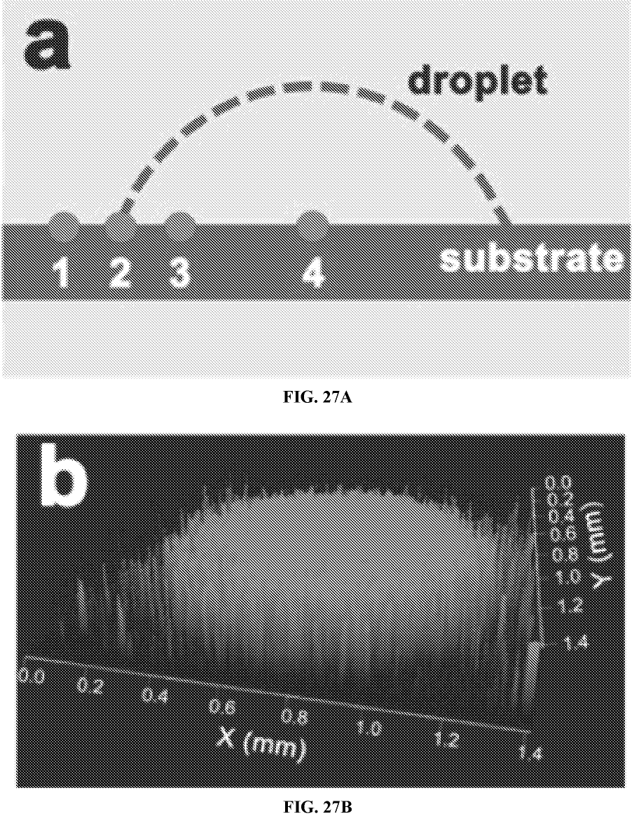

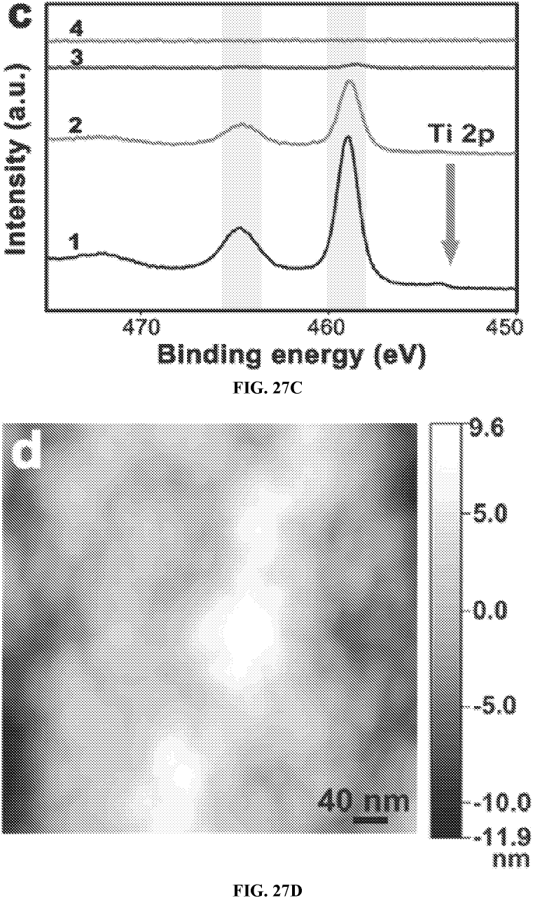

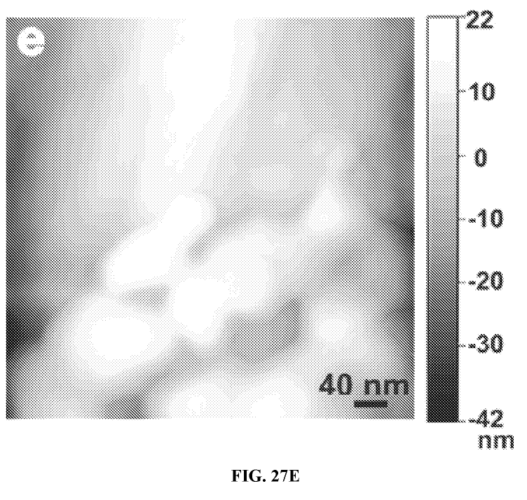

During the drop coating, a visible boundary between coated and uncoated regions developed (FIGS. 27A-27B). We formed a coating on Ti/TiO₂ having a diameter of approximately 1 mm using the MCM (MOI-N₃ = 3-N₃-7-hydroxycoumarin). The thickness of the internal coating zone was higher than that of the boundary, as indicated by an increase in XPS signals of Ti 2p (FIG. 27C). AFM mapping showed that both the boundary and internal zone were deposited with nanoaggregates in a topography similar to those reported for PDA coatings (FIGS. 27D-27E). However, nanoaggregates were much more densely packed in the internal zone of this coating.

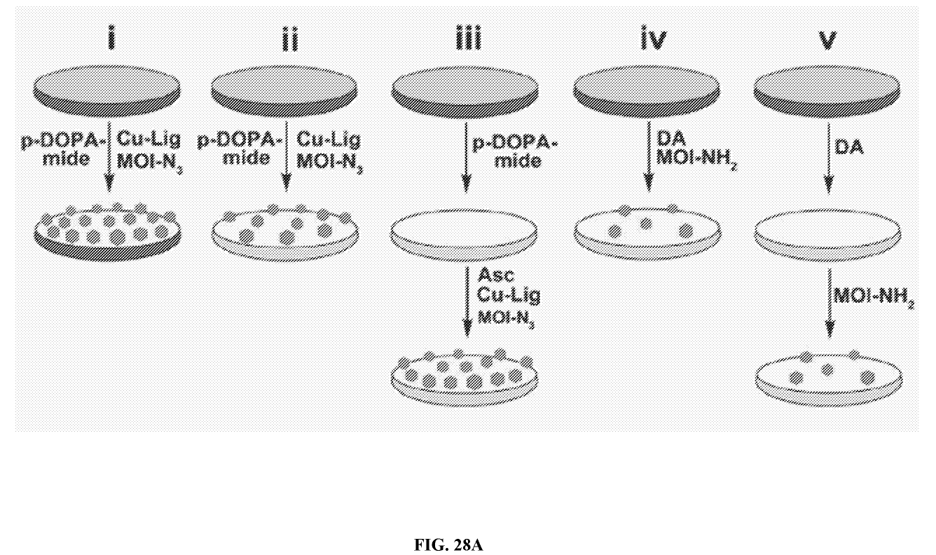

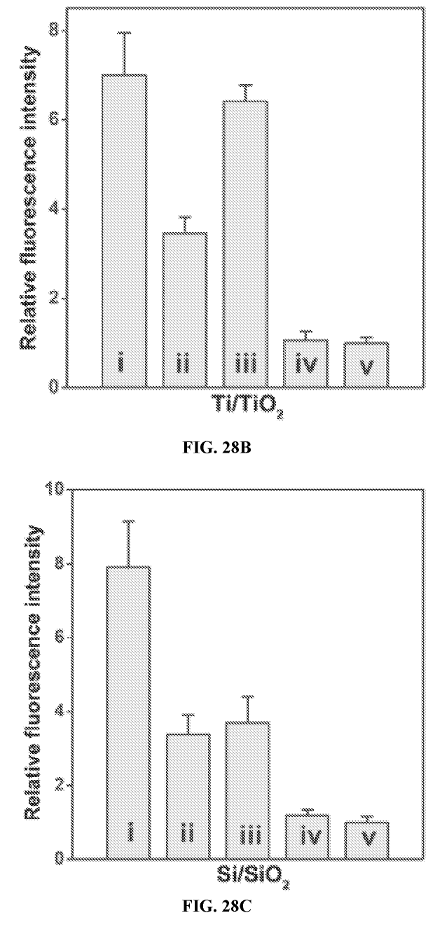

The density of surface grafting obtained by the single-step drop coating method disclosed herein (i) was compared to those obtained by several alternative representative dip coating approaches (ii-v) (FIG. 28A). To this end, Ti/TiO₂ and Si/SiO₂ surfaces were grafted with TAMRA and the relative fluorescence intensities at 561 nm excitation were measured (FIGS. 28B-28C). TAMRA-N₃ was grafted onto the substrates via click reaction in methods that employ p-DOPAmide (i-iii), while TAMRA-NH2 was grafted via Michael addition or Schiff’s base reaction in methods that employ DA (iv-v). The method of the present disclosure (i) led to the highest TAMRA density for both substrates, with fluorescence intensity approximately 2-fold higher than single-step dip coating (ii) and 6-7-fold higher than either method utilizing DA and TAMRA-NH₂ (iv and v). Method (iii) required incubation of the substrates with p-DOPAmide for significantly longer durations (3 d vs. ≤1 d) to reach grafting densities similar to those obtained in method (i).

### Example 7: Material-Independent Patterning

Patterning a solid surface with grafted molecules is critical for tissue engineering and diagnostics, but typically requires intricate microfabrication steps. The capability of the presently disclosed single-step drop coating method to produce material-independent patterning without the need for microfabrication is demonstrated herein.

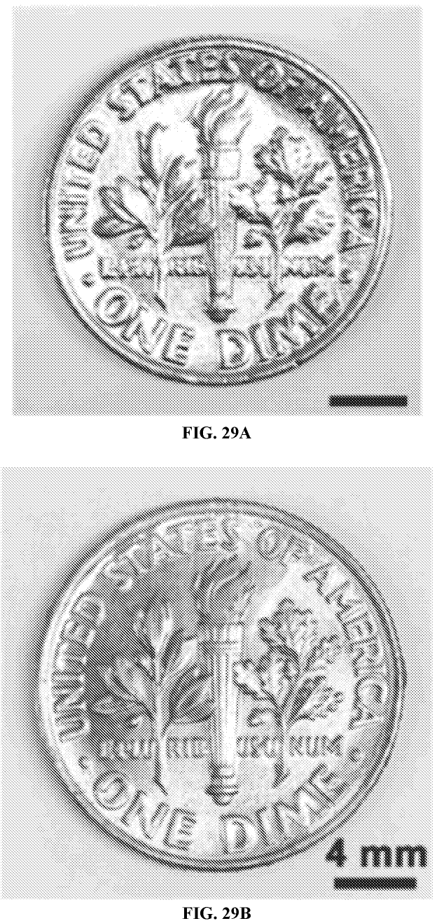

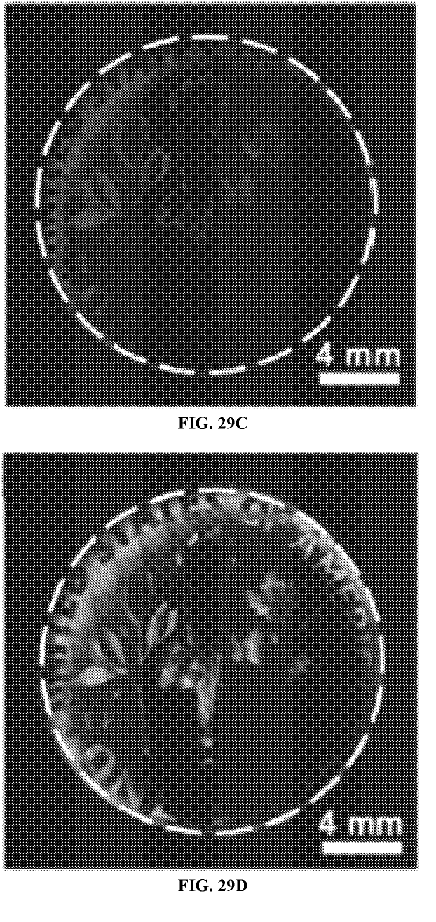

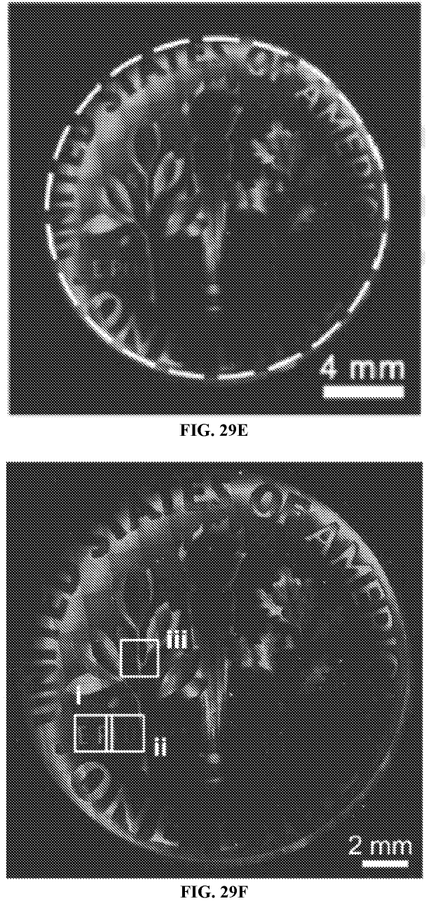

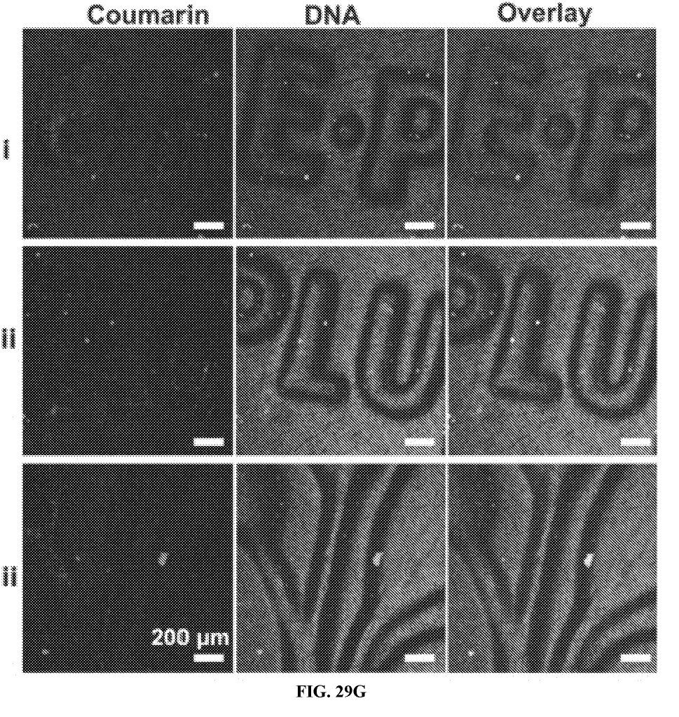

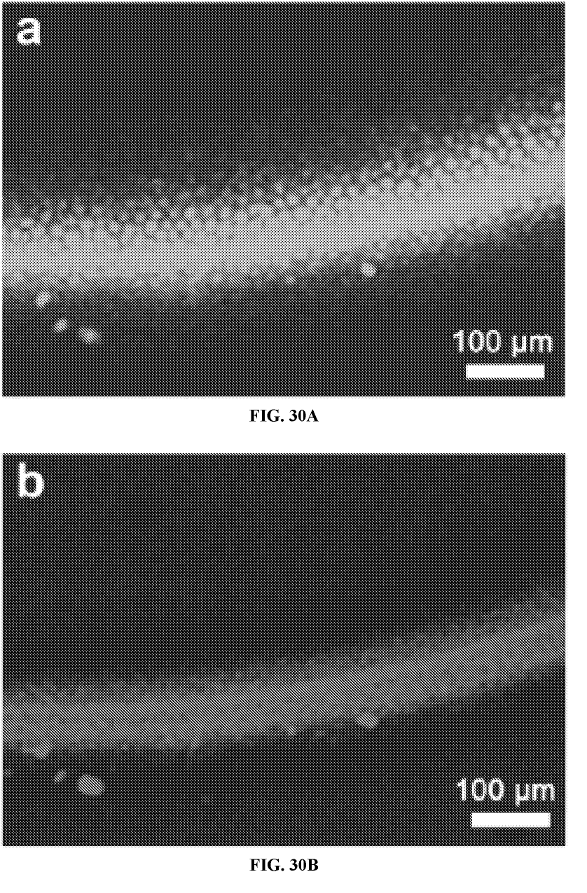

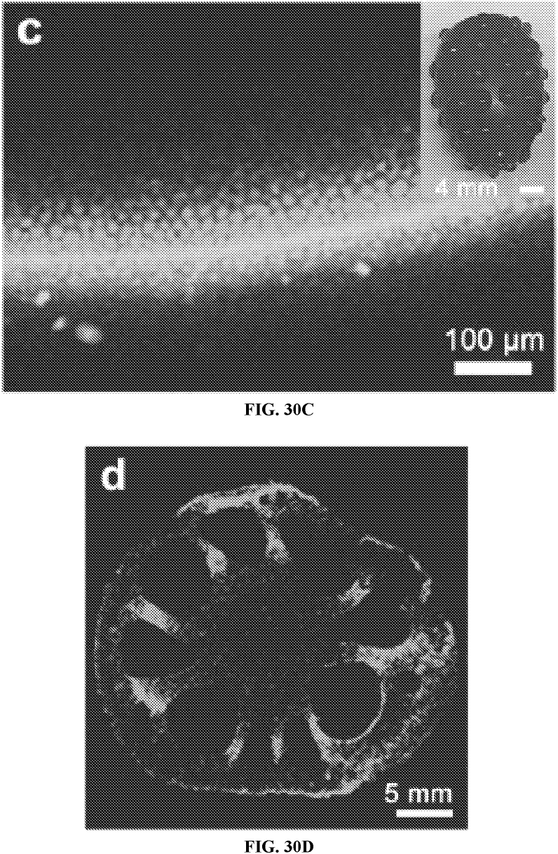

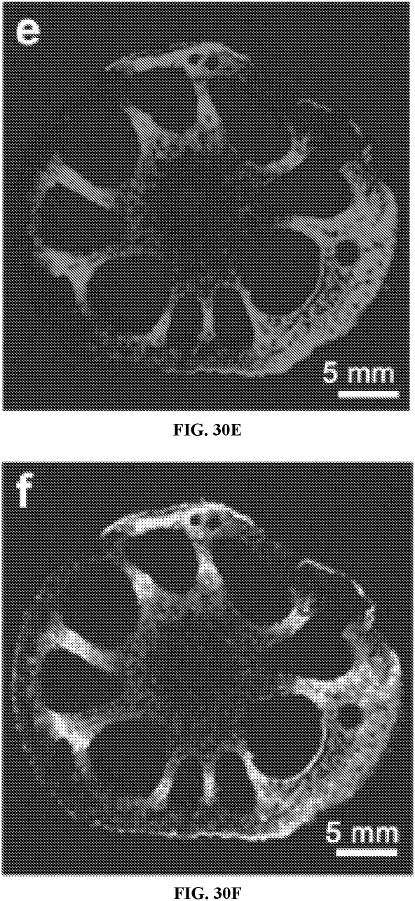

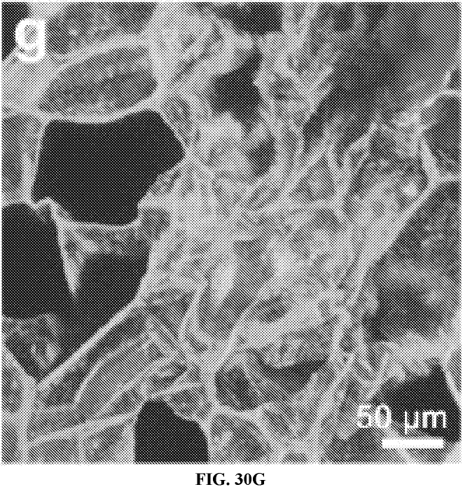

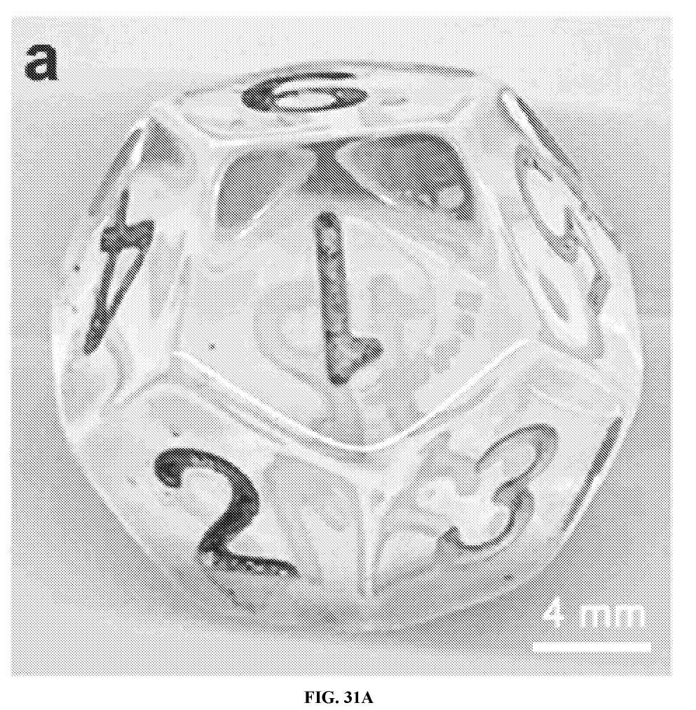

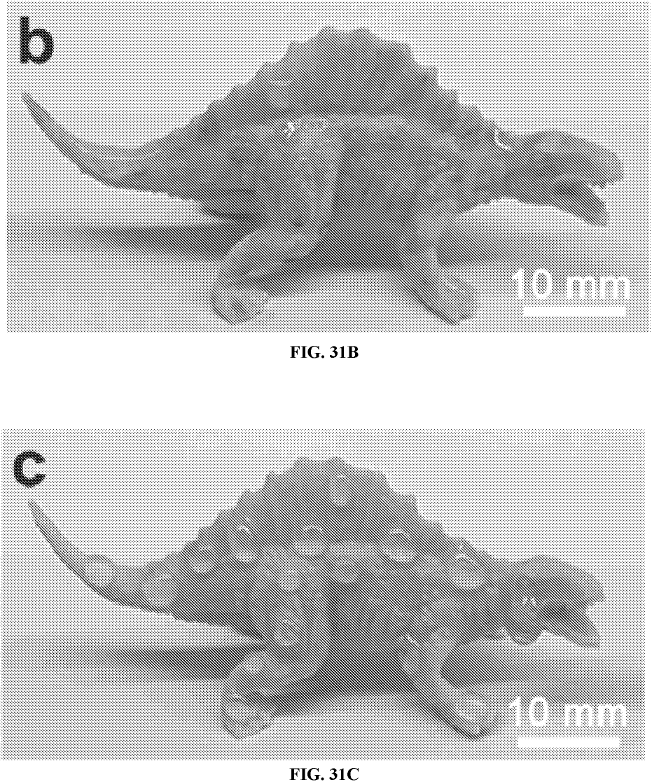

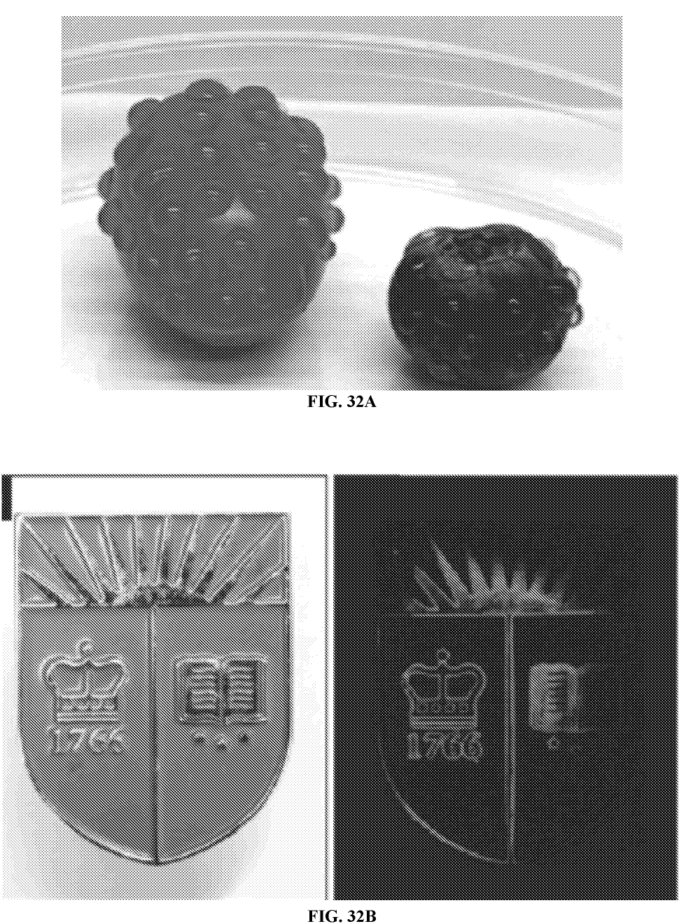

Multiplexed fluorescent patterns were generated on 3-dimensional objects, including a dime (FIGS. 29A-29G), a cherry tomato (FIGS. 30A-30C) and a lotus root (FIGS. 30D-30G) using the MCM (MOI-N₃ = 3-N₃-7-hydroxycoumarin, 0.5 mM; and N₃-DNA-FAM, 1 µM). The resulting surfaces exhibited dual fluorescence emission, through which both coumarin (405 nm excitation) and FAM (488 nm excitation) could be independently detected. These substrates are of particular value, as the cherry tomato is hydrophobic and curved, whereas the lotus root is hydrophilic and decorated with interconnecting micropores. Several structurally complex objects, which have irregular surfaces, were also drop coated. Here, the MOI-N₃ = 3-N₃-7-hydroxycoumarin MCM was used to graft numbers on a plastic polyhedral die (FIG. 31A), as well as to coat a miniature dinosaur toy (FIGS. 31B-31C), fruits (e.g. cherry tomato and blueberry; FIG. 32A), and a Rutgers university pin (FIG. 32B). All coated regions, whether flat or oblique, showed similar physical characteristics.

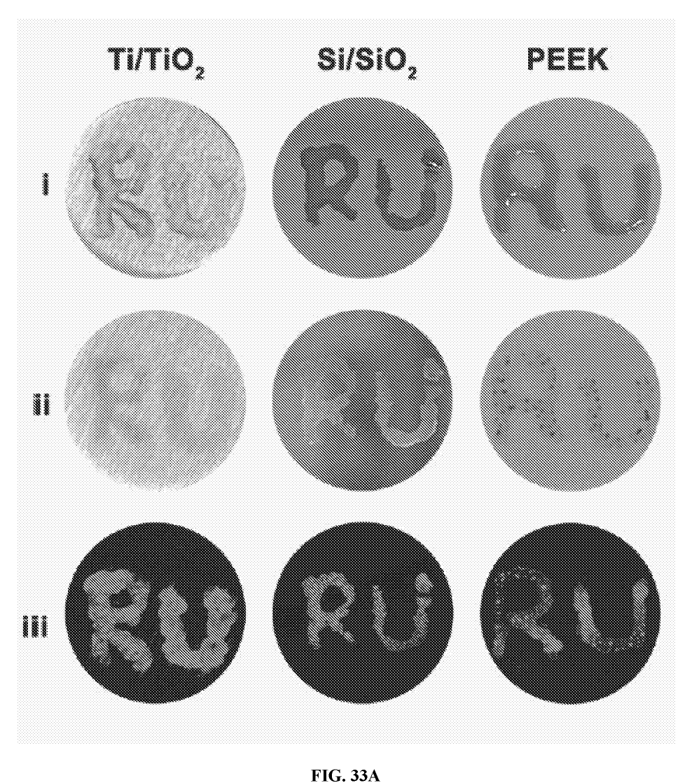

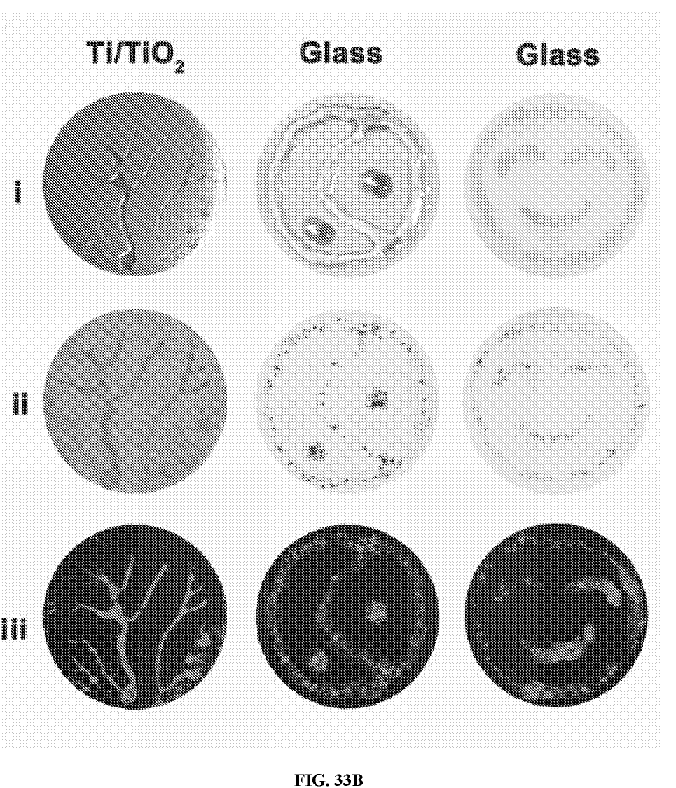

Furthermore, the single-step drop coating method of the present disclosure enables template-free patterning. Application of the MCM (MOI-N₃ = TAMRA-N₃ or 3-N₃-7-hydroxycoumarin) onto material surfaces led to high-precision drawings, as judged by both the naked eye and CLSM (FIGS. 33A-33C).

### Example 8: Surface Functionalization for Antifouling

Biofouling can cause infections at the device-tissue interface for medical devices, including biosensors, prosthetics, implants, mechanical hearts, pacemakers, catheters, and surgical tools. Inhibition of protein fouling and microbial adhesion is a critical preventative measure for these medical applications, and material surface functionalization through grafting is an attractive way to achieve this end. However, current methods have limitations with respect to stability, efficacy, and generality, and often require multi-step preparation or specialized equipment.

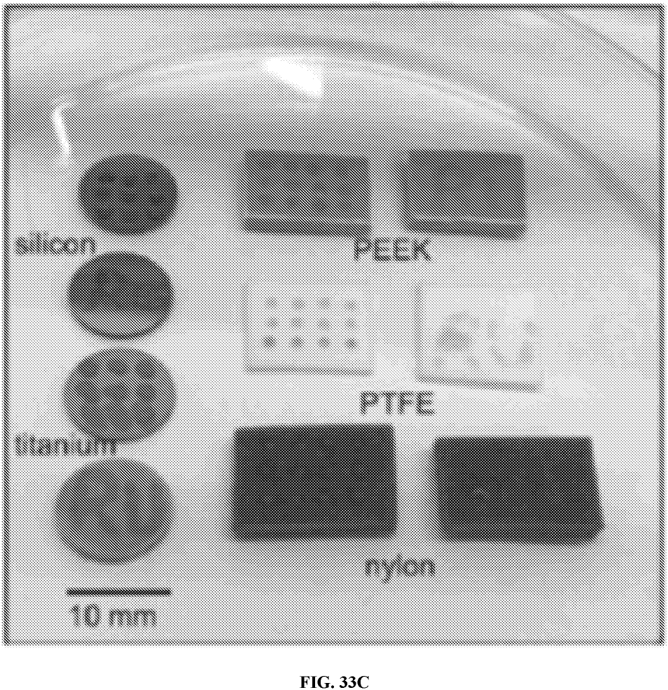

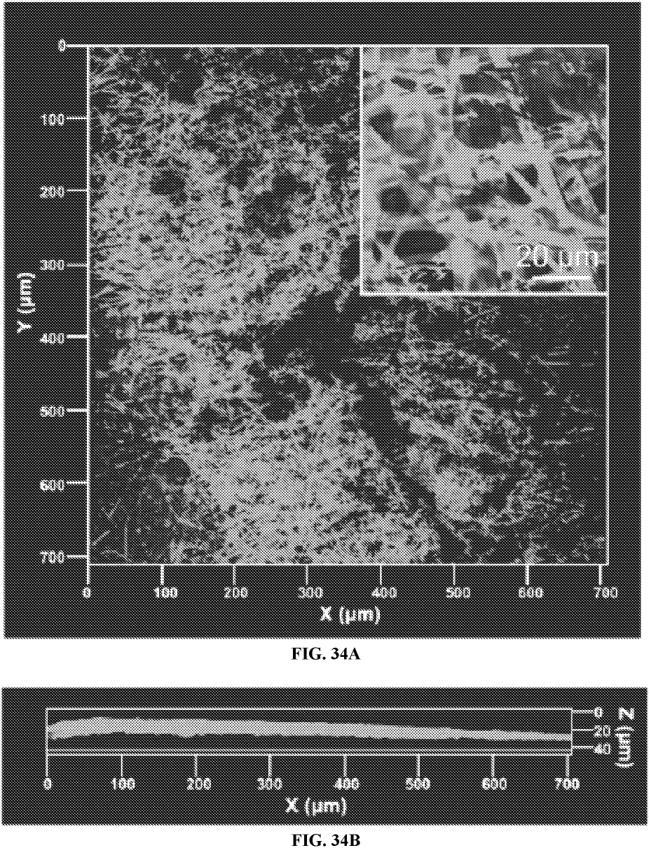

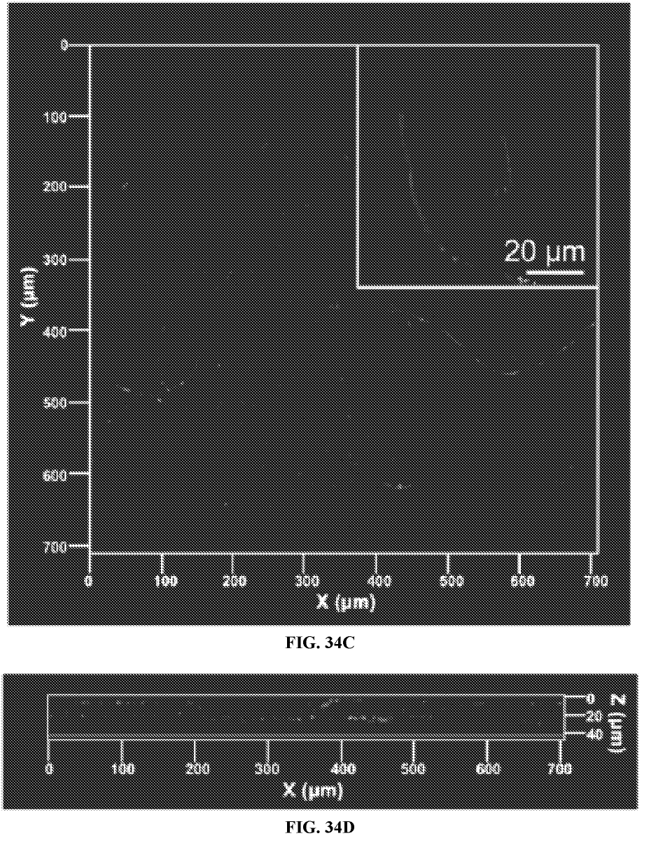

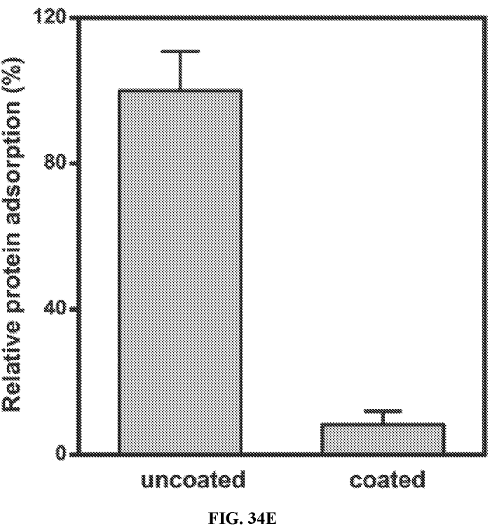

The single-step drop coating method of the present disclosure was used to functionalize surfaces with polyethylene glycol (PEG), which is known to inhibit protein fouling and bacterial adhesion when immobilized. In particular, the reduction in fouling was examined on a PEG-coated PP membrane using BSA, a serum protein that adheres to most surfaces, conjugated with fluorescein isothiocyanate (FITC-BSA) (FIGS. 34A-34E). A dense population of the protein was retained on the uncoated membranes (FIGS. 34A-34B), while the PEG-coated samples (FIGS. 34C-34D) showed a 92% decrease in protein density (FIG. 34E).

### Example 9: Surface Functionalization for Antibacterial/Antibiofilm Properties

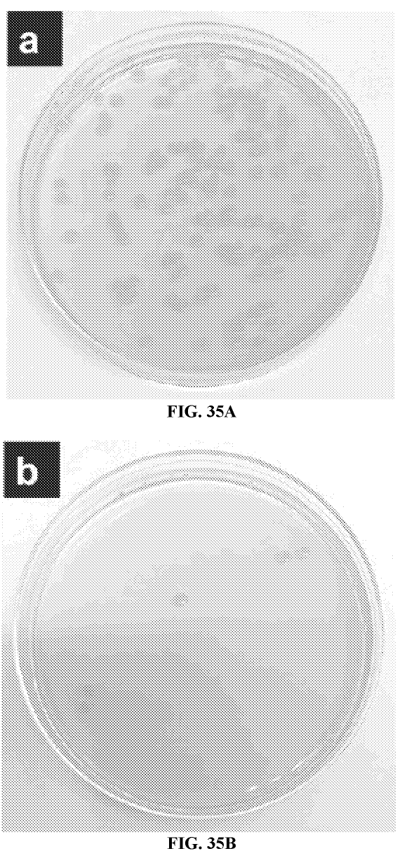

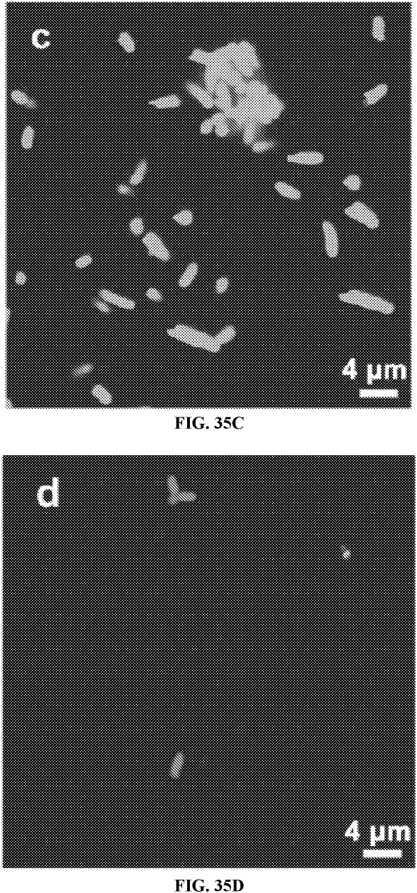

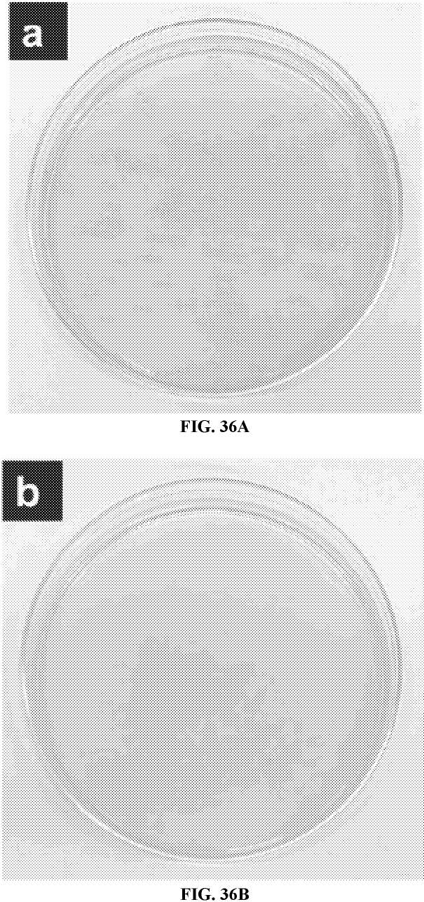

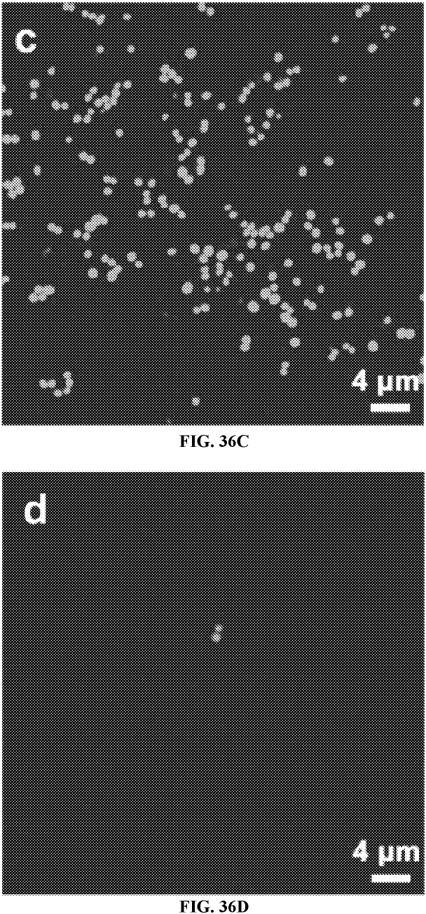

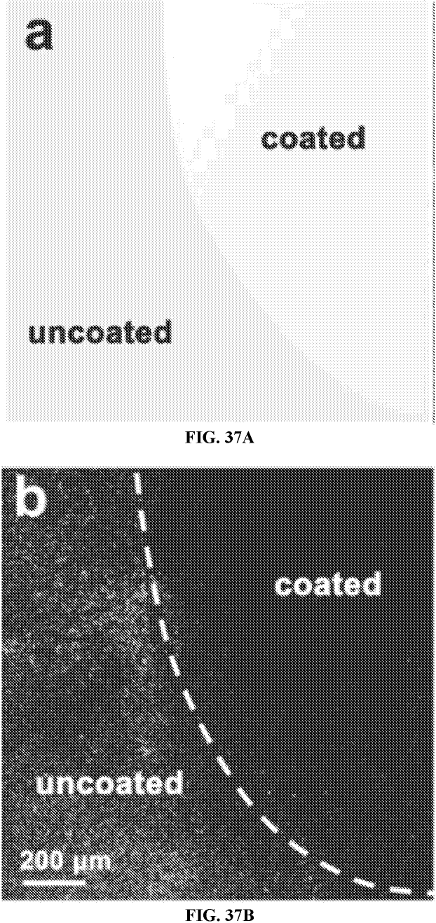

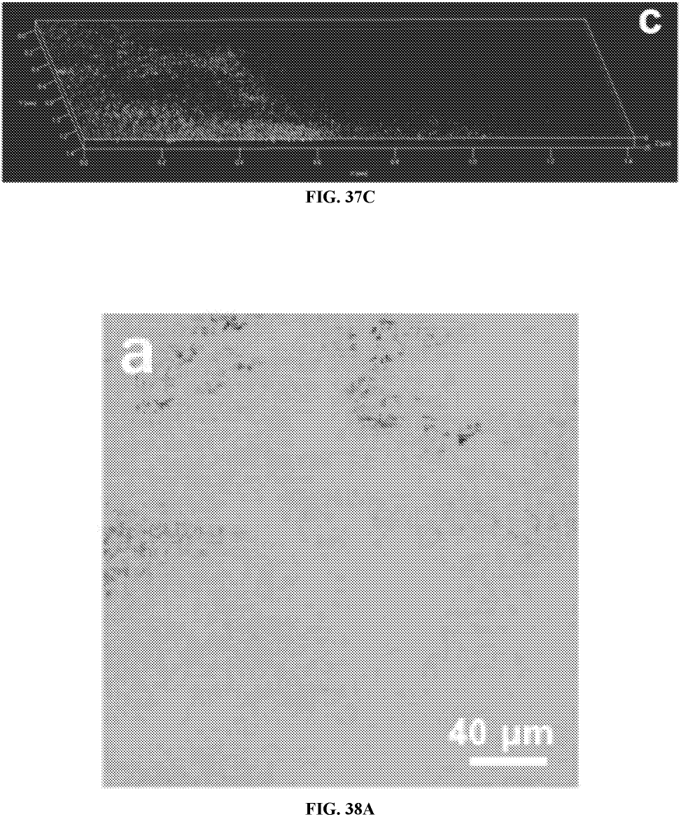

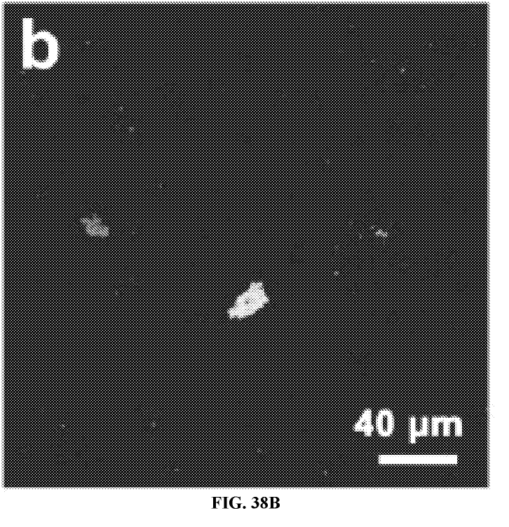

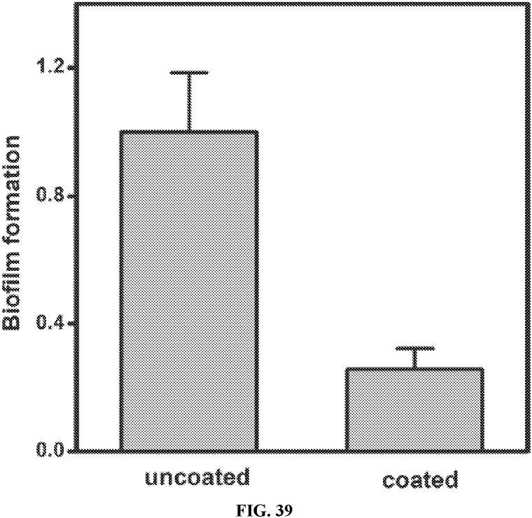

In addition to protein fouling, the adhesion of E. coli (FIGS. 35A-35D) and S. aureus (FIGS. 36A-36D) on Ti/TiO₂ substrates functionalized using the MCM (MOI-N₃ = PEG-N₃) was examined. Coated Ti/TiO₂ displayed an anti-adhesion effect against both bacterial species. Those that adhered to the substrate surface appeared to be dead, possibly due to the antimicrobial effect of the residual copper. Bacteria from both uncoated and PEG-functionalized Ti/TiO₂ were detached, cultured, and counted on agar plates (FIGS. 35A-35B and FIGS. 36A-36B). For both bacterial species, the PEG-functionalized substrates led to fewer colonies. On a substrate that had been site-specifically functionalized with PEG, S. aureus adhered mostly to the untreated region (FIGS. 37A-37C). Additionally, this method inhibited biofilm formation from S.aureus (FIGS. 38A-38B) with a 74% reduction in total biofilm mass compared to the uncoated substrate (FIG. 39).

### Example 10: Surface Functionalization for Regulating Cell Adhesion

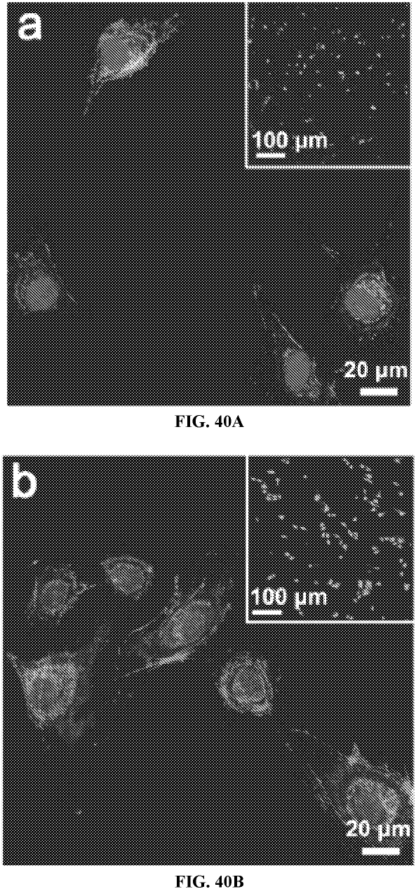

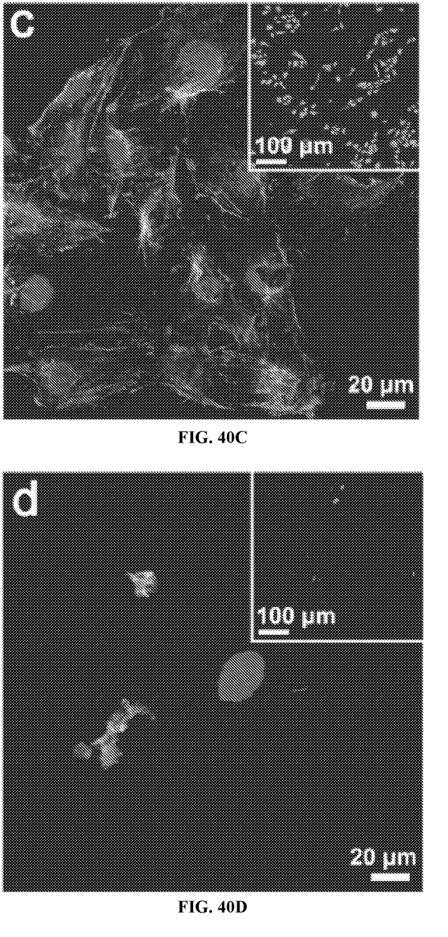

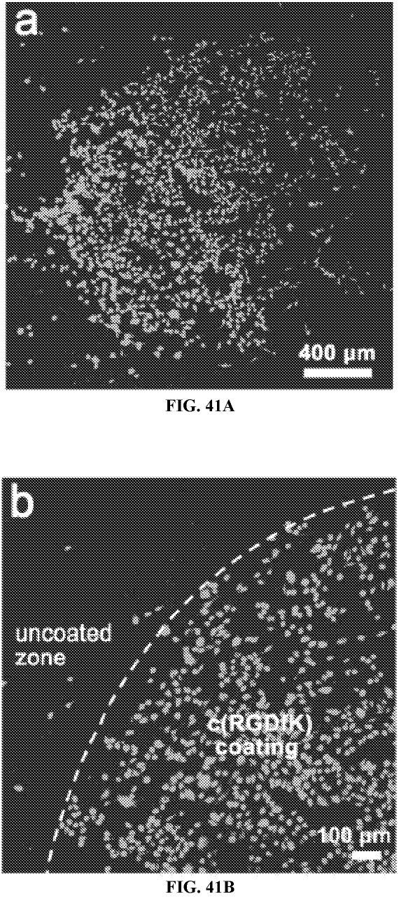

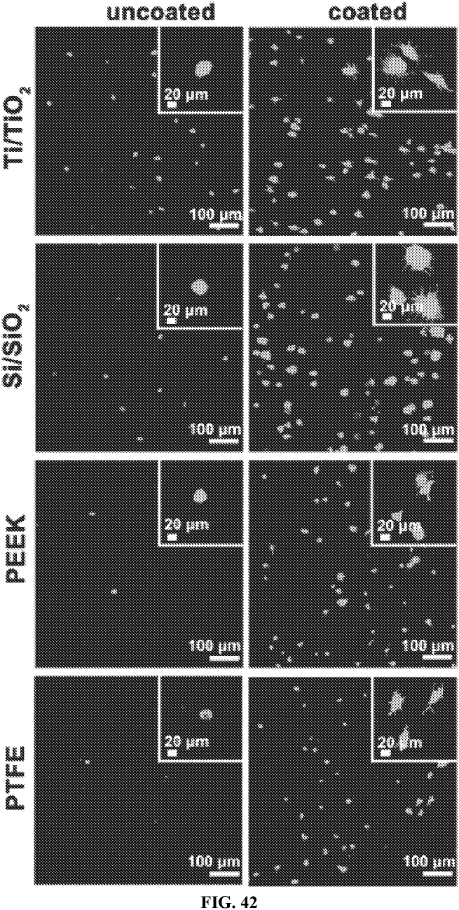

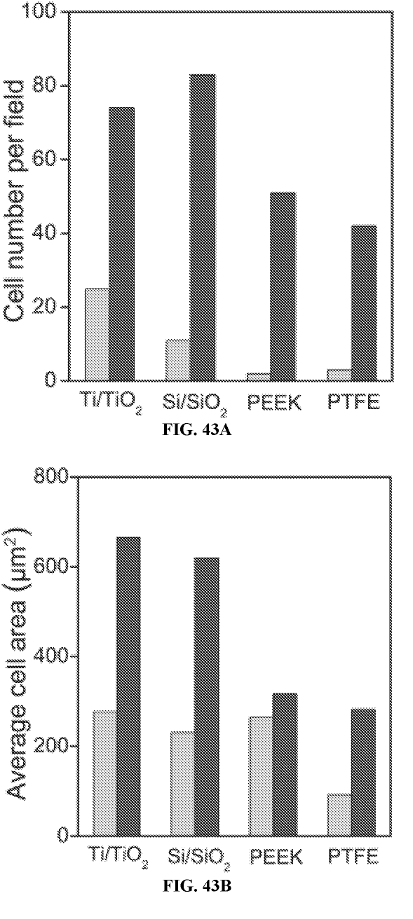

While biofouling is undesirable, the intentional adhesion of cells in culture or on implants is essential for cell viability. However, current grafting methods for regulating cell adhesion are often costly and substrate-dependent, require substrate pre-treatment (e.g. plasma cleaning or silanization), and lack site-specificity. Accordingly, use of the method of the present disclosure for immobilization of BSA and c(RGDfK), which are commonly used for the regulation of cellular adhesion, proliferation, or differentiation, was investigated. BSA facilitates the surface adsorption of fibronectin, an extracellular adhesive glycoprotein, and c(RGDfK) is a cyclic peptide bearing RGD, a ubiquitous cell adhesive motif that promotes cell attachment via integrin targeting. Ti/TiO₂ substrates were drop coated using three different MCMs (MOI-N₃ = BSA-N₃, c(RGDfK)-N₃, and PEG-N₃) and investigated their cell affinity towards human umbilical vein endothelial cells (HUVECs). The substrates grafted with BSA (FIG. 40B) and c(RGDfK) (FIG. 40C) recruited larger amounts of HUVECs and showed improved cell spreading and cytoskeleton organization compared to the uncoated surface (FIG. 40A). In contrast, substrates grafted with PEG inhibited adhesion of HUVECs, and cells that remained on the surface had a disrupted morphology and a poorly organized cytoskeleton (FIG. 40D). Adhesion of cells to site-specifically functionalized material surfaces were also investigated. HUVECs were exposed to a Ti/TiO₂ substrate that was grafted with c(RGDfK)-N₃ in one location, but with the remainder of the surface uncoated (FIGS. 41A-41B). HUVECs primarily localized onto the grafted zone. In addition, the adhesion of pre-osteoblastic cell line MC3T3-E1 on c(RGDfK)-grafted Ti/TiO₂, Si/SiO₂, PEEK, and PTFE substrates were examined (FIG. 42 and FIGS. 43A-43B). The attachment and spreading of MC3T3-E1 cells were stimulated on all substrates, including PTFE, which is known in the art to be bioinert and anti-adhesive.

### Example 11: Surface Functionalization for Tissue Engineering

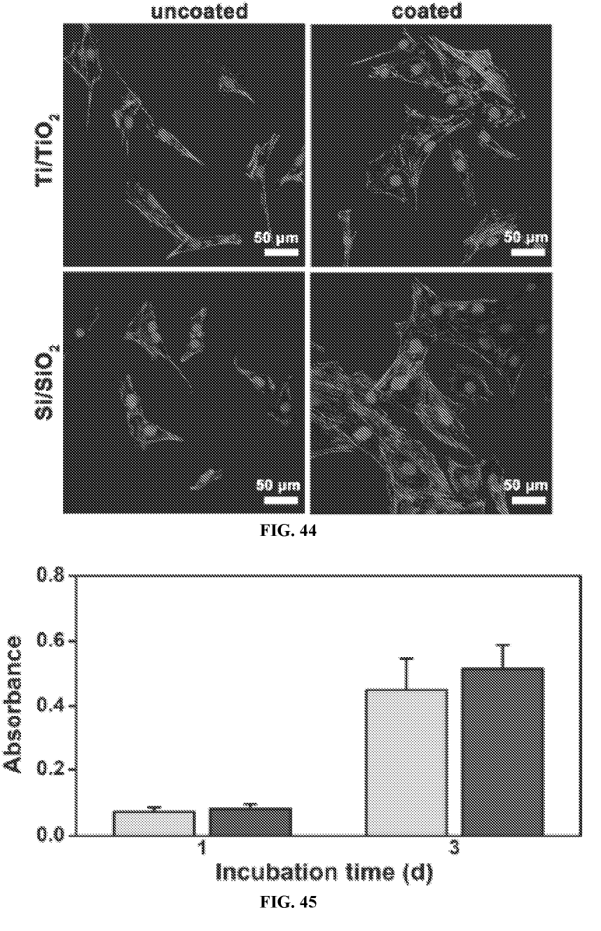

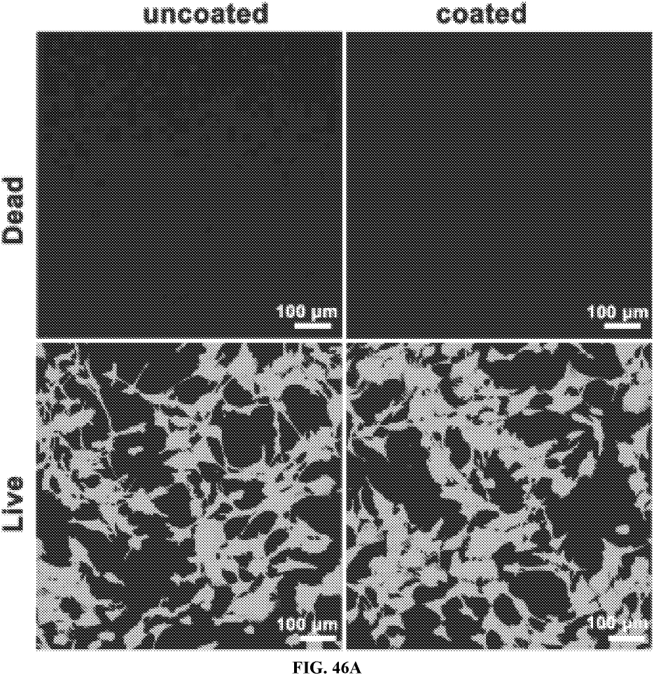

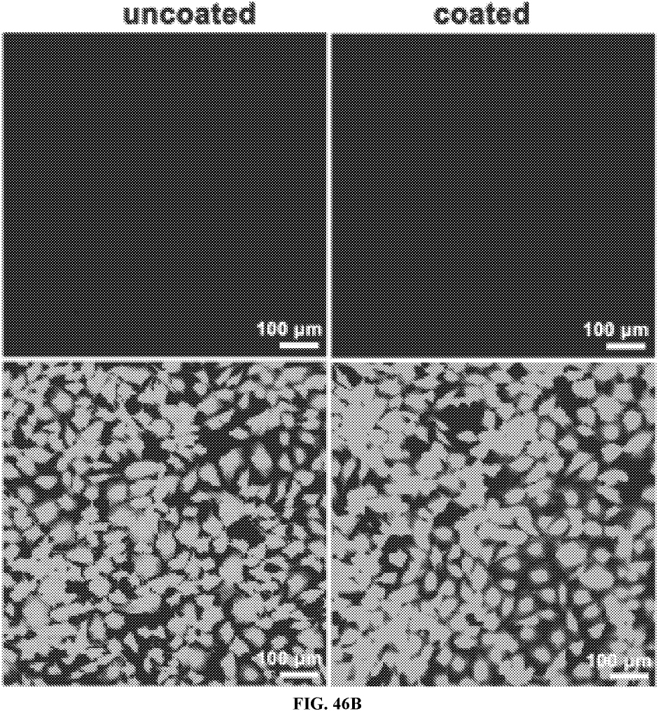

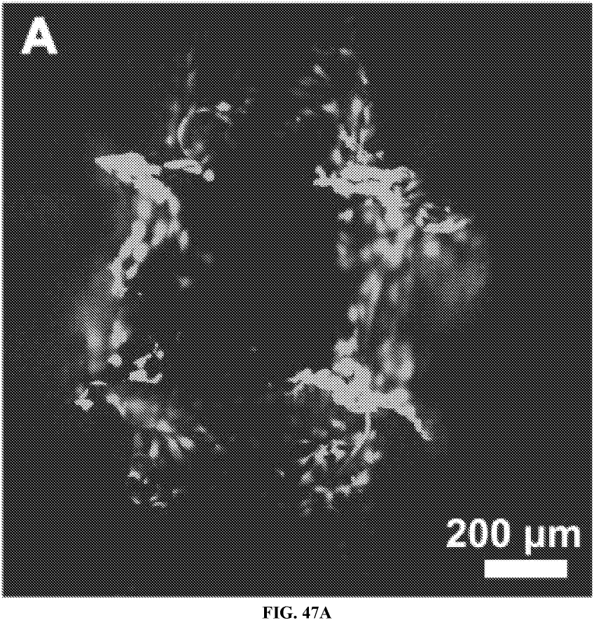

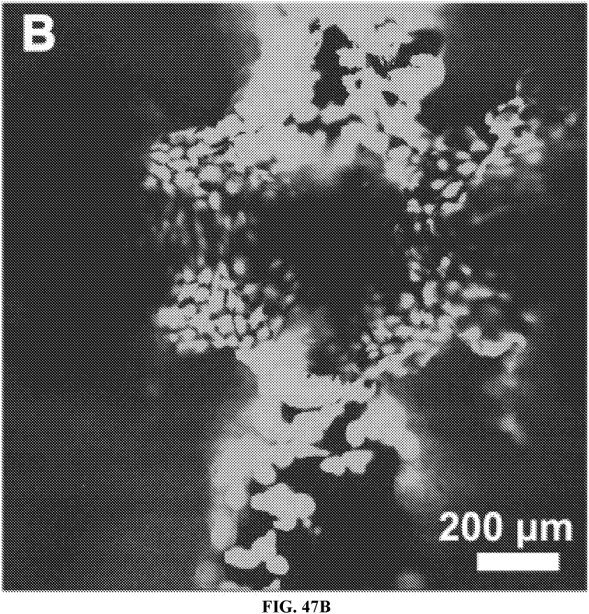

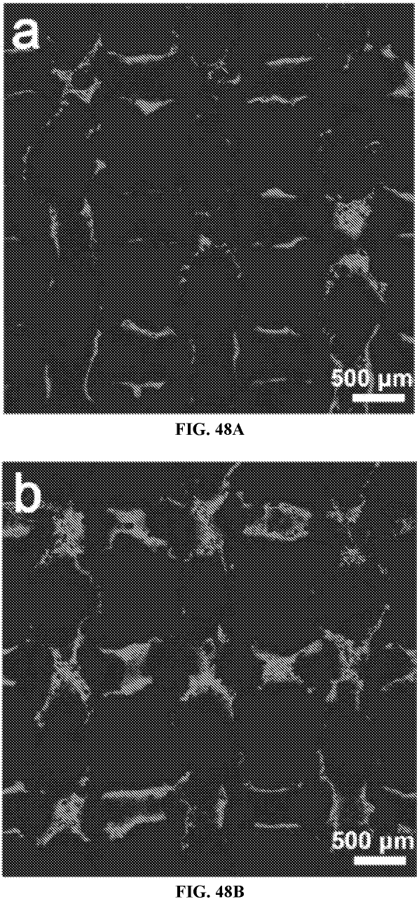

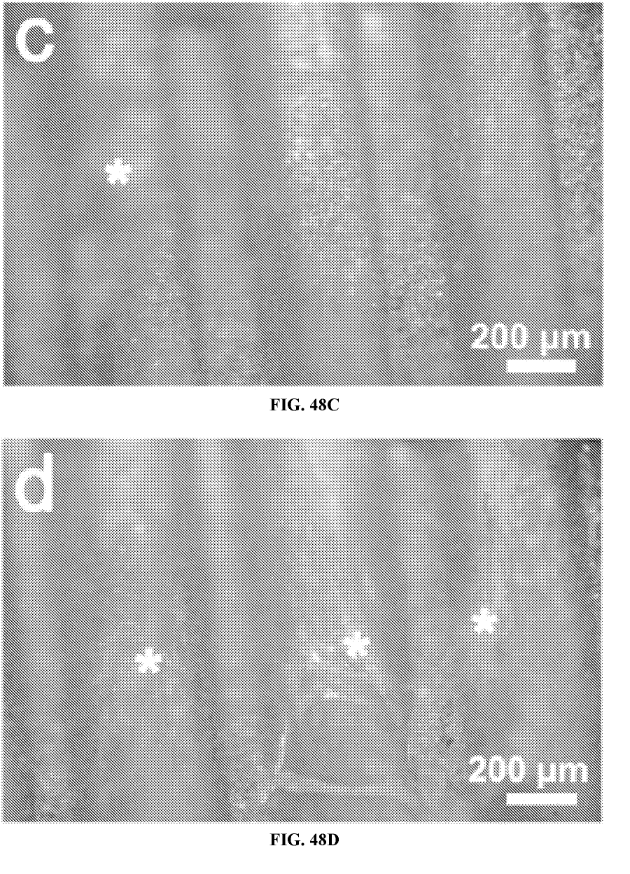

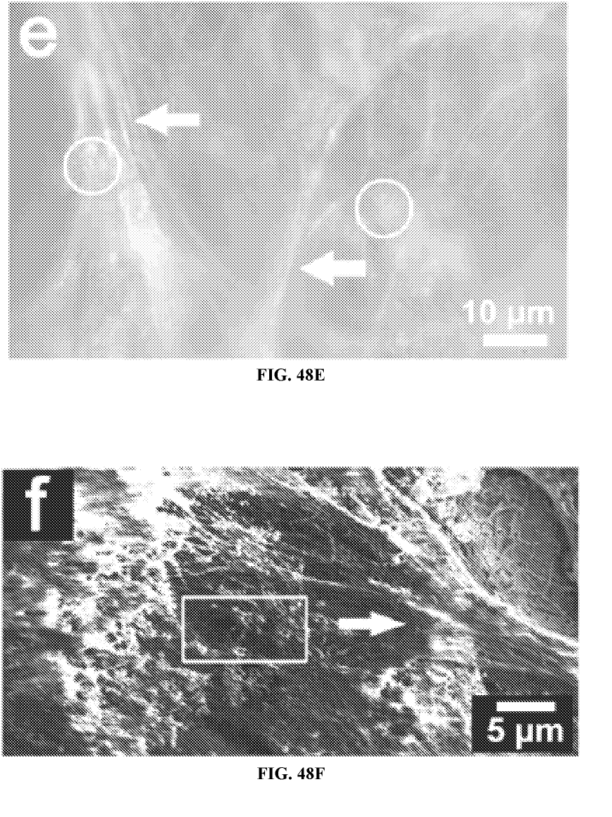

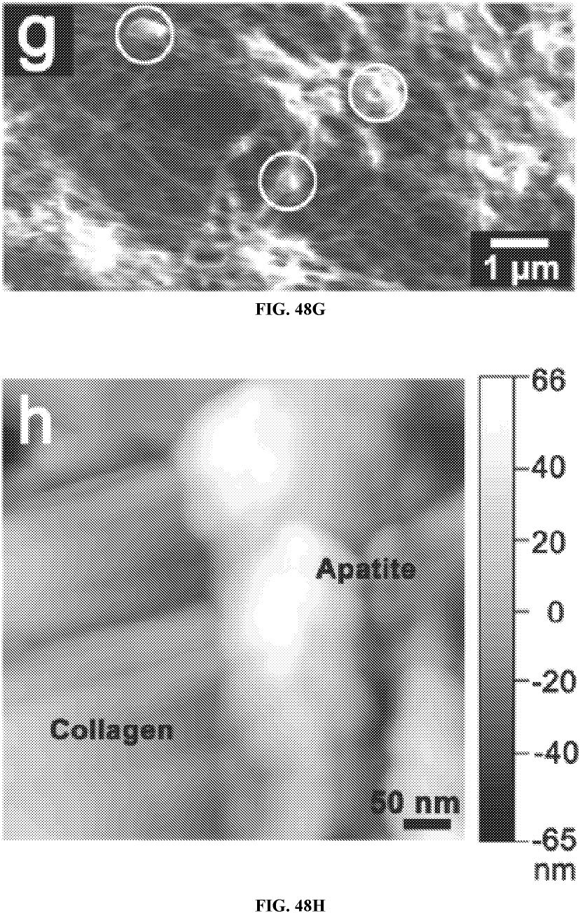

The utility of the coating method of the present disclosure was further demonstrated in tissue engineering applications. The c(RGDfK)-grafted Ti/TiO2 and Si/SiO2 promoted cytoskeleton development of MC3T3-E1 cells (FIG. 44). The MTT cell viability assay (FIG. 45) and cellular LIVE/DEAD staining assay (FIGS. 46A-46B ) showed that materials functionalized with c(RGDfK) had good cytocompatibility. Furthermore, a Ti alloy scaffold was functionalized using the MCM (MOI-N3 = c(RGDfK)-N3) and evaluated with regard to its effect on cell growth. Here, dip coating was used to coat all surfaces of the scaffold due its macroporous nature. The coated structure recruited more MC3T3-E1 cells (FIGS. 47A-47B) and showed more homogeneous cell growth (FIGS. 48A-48B) compared to the uncoated sample

Osseointegration is critical for dental and orthopedic implants, and c(RGDfK)-grafted surfaces have been shown to enhance osteoblast mineralization and bone formation. Therefore, to demonstrate utility, a Ti-based dental implant was site-selectively drop coated using the same MCM and immersed it into a culture of MC3T3-E1 cells. Bony tissue formed on the coated implant regions after as short as 4 weeks of incubation (FIGS. 48C-48D). The tissue displayed mineralized collagen fibers, characteristic of highly organized and osseous tissues (FIGS. 48E-48H).

These examples illustrate that the described surface functionalization technology has a large application scope: Manipulating the wettability, chemical stability, antifouling resistance, antimicrobial resistance, cell affinity or other cell-targeted functions of solid surfaces.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### ENUMERATED EMBODIMENTS

The following exemplary embodiments are provided, the numbering of which is not to be construed as designating levels of importance:

Embodiment 1 provides a compound of formula (I), or a salt or solvate thereof:

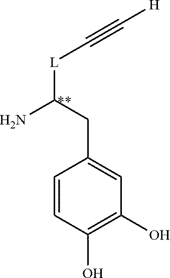

wherein: L is a linker of formula *-X-(Y)ₘ₁-Z-, wherein * is the bond between X and the carbon marked as **, wherein:

X is a bond (null), —C(═O)—, —C(═O)NH—, —C(═O)N(C_{6—10} aryl)—, —C(═O)N(C_{2—10} alkenyl)—, or —C(═O)N(C_{1—10} alkyl)—, wherein the C₆₋₁₀ aryl is optionally substituted by at least one substituent independently selected from the group consisting of halogen, —R′, —OR′, and —C(═O)OR′; each occurrence of Y is independently selected from the group consisting of —CH₂CH₂O—, —OCH₂CH₂—, and —CH₂CH₂—, wherein each CH₂ is independently optionally substituted with 1 or 2 CH₃ groups;

Z is —(CH₂)ₘ₂—, wherein each CH₂ is optionally independently substituted with 1 or 2 CH₃ groups;
each occurrence of R′ is independently hydrogen, C₂₋₅ alkenyl, or C₁₋₅ alkyl;
m1 is 0, 1, 2, 3, 4, 5, 6, 7, 8, 9, or 10;
m2 is 0, 1, 2, 3, 4, or 5;
with the proviso that L is not —C(═O)NHCH₂—.

Embodiment 2 provides the compound of Embodiment 1, wherein X is —C(═O)NH— or —C(═O)N(CH₃)—.

Embodiment 3 provides the compound of any of Embodiments 1-2, wherein at least one Y is —CH₂CH₂O— or —OCH₂CH₂—.

Embodiment 4 provides the compound of any of Embodiments 1-3, wherein L is —C(═O)NH(CH₂CH₂O)ₘ₁Z—.

Embodiment 5 provides the compound of any of Embodiments 1-4, wherein m1 is 2.

Embodiment 6 provides the compound of any of Embodiments 1-5, wherein Z is a bond or —CH₂—.

Embodiment 7 provides the compound of any of Embodiments 1-6, wherein L is selected from the group consisting of: —C(═O)NH(CH₂CH₂O)₂—, —C(═O)NH(CH₂CH₂O)₂CH₂—, and —C(═O)NH(CH₂CH₂O)CH₂—.

Embodiment 8 provides the compound of Embodiment 1, which is:

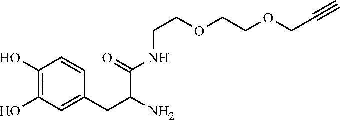 [SMILES: C#CCOCCOCCNC(=O)C(N)Cc1ccc(O)c(O)c1]

2-amino-3-(3,4-dihydroxyphenyl)-N-(2-(2-(prop-2-yn-1-yloxy)ethoxy)ethyl)propanamide, or a salt, solvate, stereoisomer, tautomer, or any mixtures thereof.

Embodiment 9 provides the compound of Embodiment 1, which is selected from the group consisting of:

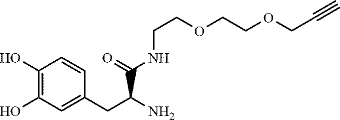 [SMILES: C#CCOCCOCCNC(=O)[C@@H](N)Cc1ccc(O)c(O)c1]

(S)-2-amino-3-(3,4-dihydroxyphenyl)-N-(2-(2-(prop-2-yn-1-yloxy)ethoxy)ethyl)propanamide; and

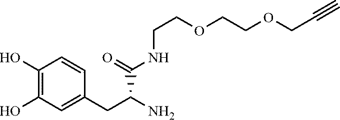 [SMILES: C#CCOCCOCCNC(=O)[C@H](N)Cc1ccc(O)c(O)c1]

(R)-2-amino-3-(3,4-dihydroxyphenyl)-N-(2-(2-(prop-2-yn-1-yloxy)ethoxy)ethyl)propanamide; or a salt, solvate, tautomer, or any mixtures thereof.

Embodiment 10 provides a composition comprising at least one compound of any of Embodiments 1-9, a copper (II) salt, a copper (I) ligand, and an azido compound (R-N₃).

Embodiment 11 provides the composition of Embodiment 10, further comprising at least one of an organocatalyst, an organometallic catalyst, a reductant, or an oxidant.

Embodiment 12 provides the composition of any of Embodiments 10-11, wherein the copper (II) salt comprises at least one selected from the group consisting of copper (II) sulfate, copper (II) chloride, copper (II) bromide, copper(II) iodide, copper(II) perchlorate, copper (II) nitrate, copper (II) hydroxide, hydrates thereof, and mixtures thereof.

Embodiment 13 provides the composition of any of Embodiments 10-12, wherein the copper (I) ligand comprises at least one selected from the group consisting of THPTA (tris(3-hydroxypropyltriazolylmethyl)amine), TBTA (tris[(1-benzyl-1H-1,2,3-triazol-4-yl)methyl]amine), BTTES (2-4-(bis-1-tert-butyl-1H-1,2,3-triazol-4yl) methylamino(methyl-1H-1,2,3-triazol-1-yl)ethanesulfonic acid), N¹-(2-(dimethylamino)ethyl)-N¹,N²,N²-trimethylethane-1,2-diamine, N¹,N^{1′}-(ethane-1,2-diyl)bis(N¹,N²,N²-trimethylethane-1,2-diamine), 2,2′-bipyridine, and combinations thereof.

Embodiment 14 provides the composition of any of Embodiments 10-13, wherein R comprises a chromophore, fluorogenic molecule, oligonucleotide, polynucleotide, nucleic acid, polyethylene glycol, peptide, polypeptide, protein, therapeutic agent, or lipid.

Embodiment 15 provides the composition of any of Embodiments 10-14, wherein the chromophore or fluorogenic molecule is covalently linked to an oligonucleotide or polynucleotide.

Embodiment 16 provides the composition of any of Embodiments 10-15, wherein the oligonucleotide or polynucleotide comprises at least two of a deoxyribonucleic acid (DNA), ribonucleic acid (RNA), or xeno nucleic acid (XNA), or any combination thereof.

Embodiment 17 provides the composition of any of Embodiments 10-16, wherein the chromophore or fluorogenic molecule is at least one selected from the group consisting of 3′,6′-dihydroxyspiro[isobenzofuran-1(3H), 9′-[9H]xanthen]-3 -one (Fluorescein), nitrobenzoxadiazole (NBD), 4,4-difluoro-4-bora-3a,4a-diaza-s-indacene (BODIPY), cyanine, rhodamine (RMA), carboxytetramethylrhodamine (TAMRA), or a derivative thereof.

Embodiment 18 provides the composition of any of Embodiments 10-14, wherein R-N₃ is selected from the group consisting of 3-N₃-7-hydroxycoumarin, TAMRA-N₃, 5′-N₃-AGCGTGACTT-3′-Fluorescein (N₃-DNA-FAM), polyethylene glycol-N₃ (PEG-N₃), cyclo[Arg—Gly—Asp—D—Phe—Lys(Azide)] (c(RGDfK)-N₃), and Bovine serum albumin with an azide modification (BSA-N₃).

Embodiment 19 provides a reaction product of the compound of any of Embodiments 1-9 and R—N₃, wherein R comprises a chromophore, fluorogenic molecule, oligonucleotide, nucleic acid, polyethylene glycol, peptide, polypeptide, protein, therapeutic agent, or lipid.

Embodiment 20 provides a polymerized product which forms upon polymerization of the composition of any of Embodiments 10-14.

Embodiment 21 provides a method of coating a surface comprising:
contacting at least a portion of the surface with a composition comprising the compound of any of Embodiments 1-9, a copper (II) salt, a copper (I) ligand, and an azido compound (R—N₃), wherein R comprises a chromophore, fluorogenic molecule, oligonucleotide, nucleic acid, polyethylene glycol, peptide, polypeptide, protein, therapeutic agent, or lipid;
wherein at least a portion of the surface is coated with a reaction product of the compound of any of Embodiments 1-9 and the azido compound to provide a surface coating.

Embodiment 22 provides the method of Embodiment 18, wherein the compound of any of Embodiments 1-9, copper (II) salt, copper (I) ligand, and azido compound are part of an aqueous mixture when contacted with the surface.

Embodiment 23 provides the method of any of Embodiments 21-22, wherein the aqueous mixture further comprises a buffer solution which is thoroughly sparged with N₂ gas.

Embodiment 24 provides the method of any of Embodiments 22-23, wherein the aqueous mixture further comprises at least one of a reductant, an oxidant, or combinations thereof.

Embodiment 25 provides the method of any of Embodiments 21-24, wherein the composition is applied to the surface by drop coating.

Embodiment 26 provides the method of any of Embodiments 21-25, wherein the surface comprises metal, stone, glass, wood, ceramic, semi-conductor, polymer, inorganic material, or combinations thereof.

Embodiment 27 provides the method of Embodiment 26, wherein the semi-conductor comprises germanium, silicon dioxide, titanium dioxide, gallium arsenide, graphene, gallium nitride, or combinations thereof.

Embodiment 28 provides the method of Embodiment 26, wherein the polymer comprises polytetrafluoroethylene, polyether ether ketone, polycarbonate, low-density polyethylene, high-density polyethylene, polypropylene, polystyrene, polyvinyl chloride, polychlorotrifluoroethylene, nylon, polysiloxane, polyethylene terephthalate, polyacrylate, polyacrylamide, polyester, polycarbonate, polyurethane, silicon rubber, or combinations thereof.

Embodiment 29 provides the method of any of Embodiments 21-28, wherein the copper (II) salt comprises at least one selected from the group consisting of copper (II) sulfate, copper (II) chloride, copper (II) bromide, copper(II) iodide, copper(II) perchlorate, copper (II) nitrate, copper (II) hydroxide, hydrates thereof, and mixtures thereof.

Embodiment 30 provides the method of any of Embodiments 21-29, wherein the copper (II) salt is copper (II) sulfate or hydrates thereof.

Embodiment 31 provides the method of any of Embodiments 21-30, wherein the copper (I) ligand comprises at least one selected from the group consisting of THPTA (tris(3-hydroxypropyltriazolylmethyl)amine), TBTA (tris[(1-benzyl-1H-1,2,3-triazol-4-yl)methyl]amine), BTTES (2-4-(bis-1-tert-butyl-1H-1,2,3-triazol-4yl) methylamino(methyl-1H-1,2,3-triazol-1-yl)ethanesulfonic acid), N¹-(2-(dimethylamino)ethyl)-N¹,N²,N²-trimethylethane-1,2-diamine, N¹,N^{1′}-(ethane-1,2-diyl)bis(N¹,N²,N²-trimethylethane-1,2-diamine), 2,2′-bipyridine, and combinations thereof.

Embodiment 32 provides the method of any of Embodiments 21-31, wherein the copper (I) ligand is THPTA (tris(3-hydroxypropyltriazolylmethyl)amine).

Embodiment 33 provides the method of any of Embodiments 21-32, wherein the chromophore or fluorogenic molecule is covalently linked to an oligonucleotide or polynucleotide.

Embodiment 34 provides the method of Embodiment 33, wherein the oligonucleotide or polynucleotide comprises at least two of a deoxyribonucleic acid (DNA), ribonucleic acid (RNA), or xeno nucleic acid (XNA), or any combinations thereof.

Embodiment 35 provides the method of any of Embodiments 33-34, wherein the chromophore or fluorogenic molecule is at least one selected from the group consisting of 3′,6′-dihydroxyspiro[isobenzofuran-1(3H),9′- [9H]xanthen]-3-one (Fluorescein), nitrobenzoxadiazole (NBD), 4,4-difluoro-4-bora-3a,4a-diaza-s-indacene (BODIPY), cyanine, rhodamine (RMA), carboxytetramethylrhodamine (TAMRA), or a derivative thereof.

Embodiment 36 provides the method of any of Embodiments 21-32, wherein R-N₃ is selected from the group consisting of 3-N₃-7-hydroxycoumarin, TAMRA-N₃,5′-N₃-AGCGTGACTT-3′-Fluorescein (N₃-DNA-FAM), polyethylene glycol-N₃ (PEG-N₃), cyclo[Arg—Gly—Asp—D—Phe—Lys(Azide)] (c(RGDfK)-N₃), and Bovine serum albumin with an azide modification (BSA-N₃).

Embodiment 37 provides the method of any of Embodiments 21-36, wherein the surface coating is gently agitated on a shaker.

Embodiment 38 provides the method of any of Embodiments 21-37, wherein the surface coating is heated at 37° C.

Embodiment 39 provides the method of any of Embodiments 21-38, wherein the surface coating is rinsed with MilliQ water.

Embodiment 40 provides the method of any of Embodiments 21-39, wherein the surface coating is dried under ambient temperature.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of formula (I), or a salt or solvate thereof: 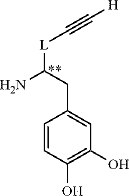
wherein:
L is a linker of formula *-X-(Y)ₘₗ-Z-, wherein * is the bond between X and the carbon marked as **, wherein:
X is a bond (null), —C(═O)—, —C(═O)NH—, —C(═O)N(C_{6—10} aryl)—, —C(═O)N(C_{2—10} alkenyl)—, or —C(═O)N(C_{1—10} alkyl)—, wherein the C₆₋₁₀ aryl is optionally substituted by at least one substituent independently selected from the group consisting of halogen, —R′, —OR′, and —C(═O)OR′;
each occurrence of Y is independently selected from the group consisting of —CH_{Z}CH₂O—, —OCH₂CH₂—, and —CH₂CH₂—, wherein each CH₂ is independently optionally substituted with 1 or 2 CH₃ groups;
Z is —(CH₂)ₘ₂—, wherein each CH₂ is optionally independently substituted with 1 or 2 CH₃ groups;
each occurrence of R′ is independently hydrogen, C₂₋₅ alkenyl, or C₁₋₅ alkyl; m1 is 0, 1,2,3,4, 5, 6, 7, 8, 9, or 10; m2 is 0, 1, 2, 3, 4, or 5;
with the proviso that L is not —C(═O)NHCH₂—.

2. The compound of claim 1, wherein X is —C(═O)NH— or —C(═O)N(CH₃)—.

3. The compound of claim 1, wherein at least one Y is —CH₂CH₂O— or —OCH₂CH₂—.

4. The compound of claim 1, wherein L is —C(═O)NH(CH₂CH₂O)ₘₗZ—.

5. The compound of claim 1, wherein m1 is 2.

6. The compound of claim 1, wherein Z is a bond or —CH₂—.

7. The compound of claim 1, wherein L is selected from the group consisting of: —C(═O)NH(CH₂CH₂O)₂—, —C(═O)NH(CH₂CH₂O)₂CH₂—, and —C(═O)NH(CH₂CH₂O)CH₂—.

8. The compound of claim 1, which is selected from the group consisting of: 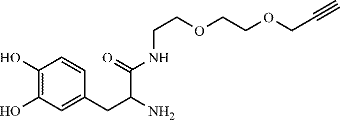 [SMILES: C#CCOCCOCCNC(=O)C(N)Cc1ccc(O)c(O)c1]
2-amino-3-(3,4-dihydroxyphenyl)-N-(2-(2-(prop-2-yn-1-yloxy)ethoxy)ethyl)propenamide; 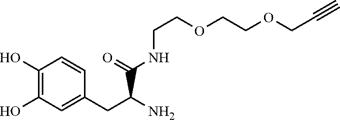 [SMILES: C#CCOCCOCCNC(=O)[C@@H](N)Cc1ccc(O)c(O)c1]
(S)-2-amino-3-(3,4-dihydroxyphenyl)-N-(2-(2-(prop-2-yn-1-yloxy)ethoxy)ethyl)propanamide; and 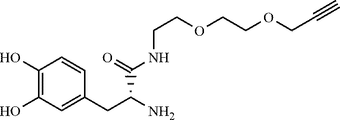 [SMILES: C#CCOCCOCCNC(=O)[C@H](N)Cc1ccc(O)c(O)c1]
(R)-2-amino-3-(3,4-dihydroxyphenyl)-N-(2-(2-(prop-2-
yn-1-yloxy)ethoxy)ethyl)propenamide;
or a salt, solvate, stereoisomer, tautomer, or any mixtures thereof.

9. (canceled)

10. A composition comprising at least one compound of claim 1, a copper (II) salt, a copper (I) ligand, and an azido compound (R-N₃),
wherein the composition optionally comprises at least one of an organocatalyst, an organometallic catalyst, a reductant, or an oxidant, and
optionally wherein at least one of the following applies:
(a) the copper (II) salt comprises at least one selected from the group consisting of copper (II) sulfate, copper (II) chloride, copper (II) bromide, copper(II) iodide, copper(II) perchlorate, copper (II) nitrate, copper (II) hydroxide, hydrates thereof, and mixtures thereof;
(b) the copper (I) ligand comprises at least one selected from the group consisting of THPTA (tris(3-hydroxypropyltriazolylmethyl)amine), TBTA (tris[(1-benzyl-1H-1,2,3-triazol-4-yl)methyl]amine), BTTES (2-4-(bis-1-tert-butyl-1H-1,2,3-triazol-4yl) methylamino(methyl-1H-1,2,3-triazol-1-yl)ethanesulfonic acid), N¹-(2-(dimethylamino)ethyl)-N¹,N²,N²-trimethylethane-1,2-diamine, N¹,N^{1′}-(ethane-1,2-diyl)bis(N¹,N²,N²-trimethylethane-1,2-diamine), 2,2′-bipyridine, and combinations thereof; and
(c) R comprises a chromophore, fluorogenic molecule, oligonucleotide, polynucleotide, nucleic acid, polyethylene glycol, peptide, polypeptide, protein, therapeutic agent, or lipid.

11-14. (canceled)

15. The composition of claim 10, wherein the chromophore or fluorogenic molecule is covalently linked to an oligonucleotide or polynucleotide, optionally wherein at least one of the following applies:
(a) the oligonucleotide or polynucleotide comprises at least two of a deoxyribonucleic acid (DNA), ribonucleic acid (RNA), or xeno nucleic acid (XNA), or any combination thereof;
(b) chromophore or fluorogenic molecule is at least one selected from the group consisting of 3′,6′-dihydroxyspiro[isobenzofuran-1(3H),9′-[9H]xanthen]-3-one (Fluorescein), nitrobenzoxadiazole (NBD), 4,4-difluoro-4-bora-3a,4a-diaza-s-indacene (BODIPY), cyanine, rhodamine (RMA), carboxytetramethylrhodamine (TAMRA), or a derivative thereof.

16-17. (canceled)

18. The composition of claim 10, wherein R-N₃ is selected from the group consisting of 3-N₃-7-hydroxycoumarin, TAMRA-N₃, 5′-N₃-AGCGTGACTT-3′-Fluorescein (N₃-DNA-FAM), polyethylene glycol—N₃ (PEG—N₃), cyclo[Arg—Gly—Asp—D—Phe—Lys(Azide)] (c(RGDfK)—N₃), and Bovine serum albumin with an azide modification (BSA—N₃).

19. A reaction product of the compound of claim 1 and R—N₃, wherein R comprises a chromophore, fluorogenic molecule, oligonucleotide, nucleic acid, polyethylene glycol, peptide, polypeptide, protein, therapeutic agent, or lipid.

20. A polymerized product which forms upon polymerization of the composition of claim 10.

21. A method of coating a surface comprising:
contacting at least a portion of the surface with a composition comprising the compound of claim 1, a copper (II) salt, a copper (I) ligand, and an azido compound (R—N₃), wherein R comprises a chromophore, fluorogenic molecule, oligonucleotide, polynucleotide, nucleic acid, polyethylene glycol, peptide, polypeptide, protein, therapeutic agent, or lipid;
wherein at least a portion of the surface is coated with a reaction product of the compound of claim 1 and the azido compound to provide a surface coating; and
optionally wherein the composition is applied to the surface by drop coating.

22. The method of claim 21, wherein the composition is part of an aqueous mixture when contacted with the surface, optionally wherein the aqueous mixture further comprises at least one of:
(a) a buffer solution which is thoroughly sparged with N₂ gas; and
(b) at least one of a reductant, an oxidant, or combinations thereof.

23-25. (canceled)

26. The method of claim 21, wherein the surface comprises metal, stone, glass, wood, ceramic, semi-conductor, polymer, inorganic material, or combinations thereof, optionally wherein at least one of the following applies:
(a) the semiconductor comprises germanium, silicon dioxide, titanium dioxide, gallium arsenide, graphene, gallium nitride, or combinations thereof; and
(b) the polymer comprises polytetrafluoroethylene, polyether ether ketone, polycarbonate, low-density polyethylene, high-density polyethylene, polypropylene, polystyrene, polyvinyl chloride, polychlorotrifluoroethylene, nylon, polysiloxane, polyethylene terephthalate, polyacrylate, polyacrylamide, polyester, polycarbonate, polyurethane, silicon rubber, or combinations thereof.

27-32. (canceled)

33. The method of claim 21, wherein the chromophore or fluorogenic molecule is covalently linked to an oligonucleotide or polynucleotide, optionally wherein the oligonucleotide or polynucleotide comprises at least two of a deoxyribonucleic acid (DNA), ribonucleic acid (RNA), or xeno nucleic acid (XNA), or any combination thereof.

34. (canceled)

35. The method of claim 33, wherein the chromophore or fluorogenic molecule is at least one selected from the group consisting of 3′,6′-dihydroxyspiro[isobenzofuran-1(3H), 9′-[9H]xanthen]-3-one (Fluorescein), nitrobenzoxadiazole (NBD), 4,4-difluoro-4-bora-3a,4a-diaza-s-indacene (BODIPY), cyanine, rhodamine (RMA), carboxytetramethylrhodamine (TAMRA), or a derivative thereof.

36. The method of claim 21, wherein R—N₃ is selected from the group consisting of 3-N₃-7-hydroxycoumarin, TAMRA-N₃, 5′-N₃-AGCGTGACTT-3′-Fluorescein (N₃-DNA-FAM), polyethylene glycol—N₃ (PEG—N₃), cyclo[Arg—Gly—Asp—D—Phe—Lys(Azide)] (c(RGDfK)—N₃), and Bovine serum albumin with an azide modification (BSA—N₃).

37. The method of claim 21, wherein at least one of the following applies:
(a) the surface coating is gently agitated on a shaker,
(b) the surface coating is heated at 37° C.;
(c) the surface coating is rinsed with MilliQ water; and
(d) the surface coating is dried under ambient temperature.

38-40. (canceled)
